SEQUENCE LISTING (14 sequences)

SEQ ID NO: 1 - protein, 179 aa, Oryza sativa
MAGSGRDRDPLVVGRVVGDVLDAFVRSTNLKVTYGSKTVSNGCELKPSMVTHQPRVEVGGNDMRTFYTLVMVDPDAPSPSDPNLREYLHWLVTDIPGTTAASFGQEVMCYESPRPTMGIHRLVFVLFQQLGRQTVYAPGWRQNFNTKDFAELYNLGSPVAAVYFNCQREAGSGGRRVYP

SEQ ID NO: 2 - protein, 256 aa, Oryza sativa
MSREENVYMAKLAEQAERYEEMVEYMEKVAKTVDVEELTVEERNLLSVAYKNVIGARRASWRIVSSIEQKEEGRGNEEHVTLIKEYRGKIEAELSKICDGILKLLDSHLVPSSTAAESKVFYLKMKGDYHRYLAEFKTGAERKEAAESTMVAYKAAQDIALADLAPTHPIRLGLALNFSVFYYEILNSPDKACNLAKQAFDEAISELDTLGEESYKDSTLIMQLLRDNLTLWTSDLTEDGGDEVKEASKGDACEGQ

SEQ ID NO: 3 - protein, 195 aa, Oryza sativa
MAMEDDEDMWANTSSPSASPPRPRGFISTALSLNSTHLQGLLPSSFVDAAASPCHASGNNNGGGDGRNAAPMSSIFFASASYHQQQHHLPAPAPLDGAILPARRFGLDMCAAAAAAPAGVPAAGDRRKRRMIKNRESAARSRARKQARVNNLETEVEQLKQENKMLRVKYEQLRKTVEVPVPVRRTLQRVLSAPF

SEQ ID NO: 4 - protein, 262 aa, Oryza sativa
MSAQAELSREENVYMAKLAEQAERYEEMVEFMEKVAKTVDSEELTVEERNLLSVAYKNVIGARRASWRIISSIEQKEESRGNEDRVTLIKDYRGKIETELTKICDGILKLLESHLVPSSTAPESKVFYLKMKGDYYRYLAEFKTGAERKDAAENTMVAYKAAQDIALAELPPTHPIRLGLALNFSVFYYEILNSPDRACNLAKQAFDEAISELDTLSEESYKDSTLIMQLLRDNLTLWTSDISEDTAEEIREAPKRDSSEGQ

SEQ ID NO: 5 - DNA, 22 bp, Artificial Sequence
  Synthesized oligo nucleotide based on Arabidopsis AP1 promoter
cttcacgagacgtcgataatca

SEQ ID NO: 6 - DNA, 19 bp, Artificial Sequence
  Primer used for PCR amplification
gcggaagagatcagggaag

SEQ ID NO: 7 - DNA, 20 bp, Artificial Sequence
  Primer used for PCR amplification
cgacaacaatcacagccaca

SEQ ID NO: 8 - DNA, 18 bp, Artificial Sequence
  Primer used for PCR amplification
caagggcgaatcaggaga

SEQ ID NO: 9 - DNA, 20 bp, Artificial Sequence
  Primer used for PCR amplification
tgaaacgataacccacagca

SEQ ID NO: 10 - DNA, 6 bp, Artificial Sequence
  C-box DNA
gacgtc

SEQ ID NO: 11 - DNA, 847 bp, Oryza sativa
tgcaccacacacagttcagctagcagatcacctagctagatagctgcctctatcacagtatatttgctccctgcaacttgctgctgctgcaatagctagcagctgcagctagtaagcaaaactataaaccttcagggttttttgcaagatcgatggccggaagtggcagggacagggaccctcttgtggttggtagggttgtgggtgatgtgctggacgcgttcgtccggagcaccaacctcaaggtcacctatggctccaagaccgtgtccaatggctgcgagctcaagccgtccatggtcacccaccagcctagggtcgaggtcggcggcaatgacatgaggacattctacacccttgtgatggtagacccagatgcaccaagcccaagtgaccctaaccttagggagtatctacattggttggtcactgatattcctggtactactgcagcgtcatttgggcaagaggtgatgtgctacgagagcccaaggccaaccatggggatccaccggctggtgttcgtgctgttccagcagctggggcgtcagacagtgtacgcgcccgggtggcgtcagaacttcaacaccaaggacttcgccgagctctacaacctcggctcgccggtcgccgccgtctacttcaactgccagcgcgaggcaggctccggcggcaggagggtctacccctagctaacgatgatcccgatcgatctgctgcatgctcactatcatcatccagcatgctatacattgcaggttcagacaattgaaatgattctcgacacacaacatatatatgatggtgtaattaattatgcaattaaatagctgagcaaggctaaggt

SEQ ID NO: 12 - DNA, 1189 bp, Oryza sativa
gccgaacaaaaagcattcgcatccacgagaccactcgaacccgacccgcctcgccgccgccgccaccgaagtaatcccttaattggtcaaaatgtctcgggaggagaatgtctacatggccaagctggccgagcaggctgaaaggtatgaggagatggttgagtacatggagaaggttgcaaagactgtagatgtggaagagctcactgttgaggagcgcaacctcttgtctgttgcttacaagaatgtgattggtgcccgccgtgcctcctggcgtattgtctcatccattgaacagaaggaggagggtcgtggcaatgaggaacatgttactctgatcaaggagtaccgtggcaagattgaagctgagctgagcaagatttgcgatggtatcctgaagttgcttgactcacaccttgtgccctcatctactgctgcagaatctaaggtgttttacctcaagatgaagggtgattaccacaggtaccttgcggaatttaagactggtgccgagagaaaggaagctgctgagagcacaatggtggcttacaaggctgctcaggatattgctctggcggatcttgctcccacccatcccataaggcttggactggcacttaacttctctgtgttctactacgagattctaaactctccagacaaggcttgcaaccttgctaagcaggcgtttgacgaagccatctccgagttggataccctcggggaggagtcttacaaggacagcactttgatcatgcagctcctgagggacaacttgaccctctggacctctgacctcacggaggacggtggtgatgaggtgaaagaagcctccaagggcgacgcctgcgagggccagtaaaatgggaagatcgatcgatcgatggctccgcatgttattggagaccatcgatttagatgcctcatgctgctgtcaccatgatggatggattcttctcctgttctactagaatgtttttcttcctgtccccccttcctctctcttctctggtttttactagggtggtagcggtcgaattagttcttcccattgctttgcatttggtgctagtggtccgtctgggctgattgttttcctctggatatgactctcgtgtgtgttgtctccagatagtgttttattgagcaatatttaaagttgtcgtccacctcctcgatgttgtttgtc

SEQ ID NO: 13 - DNA, 588 bp, Oryza sativa
atggcgatggaggacgacgaggacatgtgggcgaacacgagcagccccagcgcgtcgccgccgcggccgagggggttcatctccaccgcgctgagcctcaactcgacgcacctccaaggcctcctcccgtcgtccttcgtcgacgccgccgcctcgccgtgccacgccagcggcaacaacaacggcggcggcgacggccgcaatgccgcgccgatgtcgtccatcttcttcgcctccgcgtcgtaccaccagcagcagcaccacctccctgcccccgcgccgctcgacggcgccatcctccccgcgcgccggttcgggctcgacatgtgcgccgccgccgccgccgcccccgccggtgtccccgccgccggggaccgccgcaagaggcggatgatcaagaaccgcgagtccgcggcgaggtcccgcgcgcgcaagcaggcccgcgtcaacaacctggagaccgaggtggagcagctcaagcaggagaacaagatgctccgcgtcaagtacgagcagctgaggaagacggtggaggtgccggtgccggtgaggaggactctgcagagggtgctctccgcgccattctga

SEQ ID NO: 14 - DNA, 1155 bp, Oryza sativa
cgcctccccccaaaatttccgacgccagagcgcgaggagacgcacacagagacttggcatttgtagagtttttagatttatagatagcaaagatgtcggcacaggcggagctttcccgtgaggagaatgtgtacatggccaagctcgctgagcaagccgagaggtacgaggagatggtcgaattcatggagaaggtggccaagacggttgactctgaggagctcaccgtggaggagcgcaacctcctgtctgttgcatacaagaatgtgattggagcccgccgtgcgtcatggcgcattatctcctccattgagcagaaggaggaaagccgtggtaacgaggaccgtgtcacactcatcaaggactaccgtggcaagatcgagactgagctcaccaagatttgcgacggcattctcaagctgcttgaatcccaccttgtcccctcttccactgcccctgagtccaaggtcttctacctcaaaatgaagggtgactactacaggtaccttgccgaatttaagaccggggctgagaggaaggatgctgctgagaataccatggtggcatacaaggctgctcaggacattgctttggctgagctgcctcctactcatccaattaggcttgggctagctcttaacttctcagtgttctactatgagatcctcaactcgcctgatcgtgcttgcaacctcgcaaagcaggcttttgatgaggccatctcggagctggacaccctgagcgaggagtcctacaaggacagcactttgatcatgcaactcctccgtgataacctgaccctgtggacttcagacatctcggaggacaccgcggaagagatcagggaagctccgaagcgcgactccagcgaggggcagtaaagccggctttatgtgccctagaagcttgtagctagtgctttgctactgtgtaatgacacctatgtggctgtgattgttgtcgggaaatctggggctcccccgtatgtgaggttgctagcgatggttttgcagtctcgcctttaagctactcgtagcagagcaggtgggggtctgtggagccaggcctggttgggggtgggggagcctcttgaactgcttggtggcacttcctgtttttctgtttgcctttttattggtgtttattgttatggtc

(19) United States
(12) Patent Application Publication
(10) Pub. No.: US 2013/0019345 A1
(43) Pub. Date: Jan. 17, 2013

(54) FLORIGEN-ACTIVATING COMPLEX

(71) Applicants: Izuru Ohki, Nara (JP); Ken-ichiro Taoka, Nara (JP); Hiroyuki Tsuji, Nara (JP); Chojiro Kojima, Osaka (JP); Ko Shimamoto, Nara (JP)

(72) Inventors: Izuru Ohki, Nara (JP); Ken-ichiro Taoka, Nara (JP); Hiroyuki Tsuji, Nara (JP); Chojiro Kojima, Osaka (JP); Ko Shimamoto, Nara (JP)

(73) Assignee: NATIONAL UNIVERSITY CORPORATION NARA INSTITUTE OF SCIENCE AND TECHNOLOGY, Nara (JP)

(21) Appl. No.: 13/634,728

(22) PCT Filed: Mar. 17, 2010
(86) PCT No.: PCT/JP2011/056426

§ 371 Date: Sep. 13, 2012

(51) Int. Cl. C07K 14/415; A01H 5/00; C12N 15/63; C12N 15/29; G06F 19/16; C07K 1/107; G01N 21/64; G01N 24/08; C12Q 1/68; C12N 15/82; G01N 33/53

(52) U.S. Cl. 800/290; 800/298; 435/320.1; 536/23.6; 436/501; 530/370; 435/6.13; 703/11

(57) ABSTRACT

Provided is a crystal of a florigen activation complex, including a florigen, a 14-3-3 protein, and a bZIP transcription factor bound to each other. In addition, flowering of a plant is regulated by controlling mechanisms of interactions among those proteins utilizing the crystal. These are achieved as follows. With attention focused on the fact that Hd3a is bound to FD1 via GF14c to form a florigen activation complex, a crystal of the florigen activation complex is produced, conformational information is obtained through the use of the crystal of the florigen activation complex, and the flowering of a plant is regulated by controlling mechanisms of interactions among the florigen and the like utilizing such conformational information.

### TECHNICAL FIELD

The present invention relates to a crystal of a florigen activation complex, including a florigen bound to a bZIP transcription factor via a 14-3-3 protein, and to uses of conformational information on the florigen activation complex obtained from the crystal, and a function of the florigen activation complex.

The present application claims priority from Japanese Patent Application No. 2010-061562, which is incorporated herein by reference.

### BACKGROUND ART

Regulation of flowering of plants under various environments is considered to be effective for realizing stable food production and establishment of a recycling-oriented economic society system utilizing plant biomass and the like. A molecular basis for signal transduction of a plant environment response needs to be elucidated in order to establish a technology for regulating flowering of plants.

It was proposed 70 years ago that a florigen was present as an inducer of flowering of plants. In recent years, it has been clarified that the molecular nature for the florigen is a product of an FT gene universally present in higher plants. The florigen is a molecule which is synthesized in leaves when day length becomes optimum for flower-bud formation, moves to the shoot apex, and initiates flowering. In rice, Hd3a corresponds to the florigen and is considered to promote heading, and there has been proposed use of a rice Hd3a gene for regulating flowering of plants (Patent Literature 1 and Non Patent Literature 1).

There is a report that rice Hd3a interacts with a rice 14-3-3 protein GF14c, thereby suppressively controlling flowering (Non Patent Literature 2). In Non Patent Literature 2, it has been found that the rice 14-3-3 protein GF14c interacts with Hd3a, as a result of screening of a protein which interacts with Hd3a by a yeast two-hybrid method. It has been suggested that GF14c is mainly localized in the cytoplasm and a complex of Hd3a and GF14c is mainly present in the cytoplasm. GF14c has been considered to act as a negative regulator of flowering based on, for example, the fact that overexpression of GF14c leads to a delay in flowering.

In Arabidopsis, it is considered that an FT protein as the molecular nature for the florigen interacts with a bZIP transcription factor FD and the like in the nucleus, contributing to expression of floral meristem identity genes (Non Patent Literatures 3 and 4). There is a report on use of a bZIP transcription factor for controlling flowering (Patent Literature 2).

Although there are many research reports on the florigen, an action mechanism of the florigen in floral induction has not been clarified yet.

### CITATION LIST

### Patent Literature

[PTL 1] JP 2002-153283 A
[PTL 2] JP 2003-274972 A

### Non Patent Literature

[NPL 1] Tamaki S et al. Science (2007) vol. 316 (5827) pp. 1033-6
[NPL 2] Purwestri Y A et al. Plant and Cell Physiology 2009 50(3): 429-438
[NPL 3] Wigge et al. Science (2005) vol. 309 (5737) pp. 1056-9
[NPL 4] Abe et al. Science (2005) vol. 309 (5737) pp. 1052-6

### SUMMARY OF INVENTION

### Technical Problem

An object of the present invention is to provide a crystal of a florigen activation complex, including a florigen bound to a bZIP transcription factor via a 14-3-3 protein. Another object of the present invention is to regulate flowering of a plant by clear lying mechanisms of interactions among those proteins utilizing the crystal and controlling such interactions.

### Solution to Problem

The inventors of the present invention have made extensive studies in order to achieve the objects. As a result, the inventors have succeeded in crystallizing a florigen activation complex, including a florigen bound to a bZIP transcription factor via a 14-3-3 protein, and have found that the flowering of a plant can be regulated by controlling an interaction mechanism utilizing conformational information obtained from the crystal of the florigen activation complex. Thus, the present invention has been completed.

That is, the present invention includes the following items.

1. A method of regulating flowering of a plant, the method including promoting and/or suppressing formation of a florigen activation complex including a complex of a florigen, a 14-3-3 protein, and a bZIP transcription factor by affecting a binding site between the florigen and the 14-3-3 protein and/or a binding site between the 14-3-3 protein and the bZIP transcription factor in the florigen activation complex,

in which:

the binding site between the florigen and the 14-3-3 protein includes at least one site selected from the group consisting of sites of the florigen corresponding to D62, M63, R64, P96, T98, F103, and R132 in SEQ ID NO: 1 and sites of the 14-3-3 protein corresponding to F200, D201, I204, E212, Y215, R226, and D227 in SEQ ID NO: 2; and

the binding site between the 14-3-3 protein and the bZIP transcription factor includes at least one site selected from the group consisting of sites of the 14-3-3 protein corresponding to K51, R58, F121, R131, L224, and D227 in SEQ ID NO: 2 and sites of the bZIP transcription factor corresponding to R189 to F195 in SEQ ID NO: 3.

2. A method of regulating flowering of a plant according to the above-mentioned item 1, in which the promoting and/or suppressing of the formation of the florigen activation complex includes generating a transformant including at least one of the following proteins (A) to (C):
(A) a florigen having a mutation in at least one binding site between the florigen and a 14-3-3 protein;
(B) a 14-3-3 protein having a mutation in at least one binding site between a florigen and the 14-3-3 protein and/or at least one binding site between the 14-3-3 protein and a bZIP transcription factor; and
(C) a bZIP transcription factor having a mutation in at least one binding site between a 14-3-3 protein and the bZIP transcription factor.
3. A transformant, including at least one of the following proteins (A) to (C):
(A) a florigen having a mutation in at least one binding site between the florigen and a 14-3-3 protein;
(B) a 14-3-3 protein having a mutation in at least one binding site between a florigen and the 14-3-3 protein and/or at least one binding site between the 14-3-3 protein and a bZIP transcription factor; and
(C) a bZIP transcription factor having a mutation in at least one binding site between a 14-3-3 protein and the bZIP transcription factor,

in which:

the binding site between the florigen and the 14-3-3 protein includes at least one site selected from the group consisting of sites of the florigen corresponding to D62, M63, R64, P96, T98, F103, and R132 in SEQ ID NO: 1 and sites of the 14-3-3 protein corresponding to F200, D201, I204, E212, Y215, R226, and D227 in SEQ ID NO: 2; and

the binding site between the 14-3-3 protein and the bZIP transcription factor includes at least one site selected from the group consisting of sites of the 14-3-3 protein corresponding to K51, R58, F121, R131, L224, and D227 in SEQ ID NO: 2 and sites of the bZIP transcription factor corresponding to R189 to F195 in SEQ ID NO: 3.

4. A polynucleotide, which encodes at least one of the following proteins (A) to (C):
(A) a florigen having a mutation in at least one binding site between the florigen and a 14-3-3 protein;
(B) a 14-3-3 protein having a mutation in at least one binding site between a florigen and the 14-3-3 protein and/or at least one binding site between the 14-3-3 protein and a bZIP transcription factor; and
(C) a bZIP transcription factor having a mutation in at least one binding site between a 14-3-3 protein and the bZIP transcription factor,

in which:

the binding site between the florigen and the 14-3-3 protein includes at least one site selected from the group consisting of sites of the florigen corresponding to D62, M63, R64, P96, T98, F103, and R132 in SEQ ID NO: 1 and sites of the 14-3-3 protein corresponding to F200, D201, I204, E212, Y215, R226, and D227 in SEQ ID NO: 2; and

the binding site between the 14-3-3 protein and the bZIP transcription factor includes at least one site selected from the group consisting of sites of the 14-3-3 protein corresponding to K51, R58, F121, R131, L224, and D227 in SEQ ID NO: 2 and sites of the bZIP transcription factor corresponding to R189 to F195 in SEQ ID NO: 3.

5. A recombinant vector, including at least one of the polynucleotides of the above-mentioned item 4.
6. A method of screening a substance that regulates flowering of a plant, the method including any one of the following steps:
(1) a step including bringing a candidate substance into contact with any one of a florigen and a 14-3-3 protein, and bringing the candidate substance into contact with any one of the 14-3-3 protein and the florigen, respectively; and
(2) a step including bringing a candidate substance into contact with any one of a 14-3-3 protein to which a florigen is bound or unbound and a bZIP transcription factor, and bringing the candidate substance into contact with any one of the bZIP transcription factor and the 14-3-3 protein to which a florigen is bound or unbound, respectively.
7. A method of screening a substance that regulates flowering of a plant according to the above-mentioned item 6, the method further including the following step of:

selecting a candidate substance that promotes and/or inhibits binding in a binding site between the florigen and the 14-3-3 protein and/or a binding site between the 14-3-3 protein and the bZIP transcription factor in the presence of the candidate substance,

in which:

the binding site between the florigen and the 14-3-3 protein includes at least one site selected from the group consisting of sites of the florigen corresponding to D62, M63, R64, P96, T98, F103, and R132 in SEQ ID NO: 1 and sites of the 14-3-3 protein corresponding to F200, D201, I204, E212, Y215, R226, and D227 in SEQ ID NO: 2; and

the binding site between the 14-3-3 protein and the bZIP transcription factor includes at least one site selected from the group consisting of sites of the 14-3-3 protein corresponding to K51, R58, F121, R131, L224, and D227 in SEQ ID NO: 2 and sites of the bZIP transcription factor corresponding to R189 to F195 in SEQ ID NO: 3.

8. A method of screening a substance that regulates flowering of a plant according to the above-mentioned item 6 or 7,

in which:

the florigen includes a florigen polypeptide fragment including an amino acid sequence which includes at least a sequence of amino acids at positions 62 to 132, starts with one of amino acids at positions 1 to 6, and ends with one of amino acids at positions 165 to 177, in an amino acid sequence set forth in SEQ ID NO: 1;

the 14-3-3 protein includes a 14-3-3 protein polypeptide fragment including an amino acid sequence which includes at least a sequence of amino acids at positions 51 to 227, starts with one of amino acids at positions 1 to 5, and ends with one of amino acids at positions 230 to 256, in an amino acid sequence set forth in SEQ ID NO: 2; and

the bZIP transcription factor includes a bZIP transcription factor polypeptide fragment including an amino acid sequence which includes at least a sequence of amino acids at positions 189 to 195, starts with one of amino acids at positions 182 to 188, and ends with the amino acid at position 195, in an amino acid sequence set forth in SEQ ID NO: 3.

9. A polypeptide fragment, which is selected from the following:
(i) a novel florigen polypeptide fragment including an amino acid sequence which includes at least a sequence of amino acids at positions 62 to 132, starts with one of amino acids at positions 1 to 6, and ends with one of amino acids at positions 165 to 177, in an amino acid sequence set forth in SEQ ID NO: 1;
(ii) a novel 14-3-3 protein polypeptide fragment including an amino acid sequence which includes at least a sequence of amino acids at positions 51 to 227, starts with one of amino acids at positions 1 to 5, and ends with one of amino acids at positions 230 to 256, in an amino acid sequence set forth in SEQ ID NO: 2; and
(iii) a novel bZIP transcription factor polypeptide fragment including an amino acid sequence which includes at least a sequence of amino acids at positions 189 to 195, starts with one of amino acids at positions 182 to 188, and ends with the amino acid at position 195, in OsFD1 set forth in SEQ ID NO: 3.
10. A polynucleotide, which encodes any one of the polypeptide fragments (i) to (iii) of the above-mentioned item 9.
11. A florigen activation complex, including a florigen polypeptide fragment bound to a bZIP transcription factor polypeptide fragment via a 14-3-3 protein polypeptide fragment,

in which the florigen polypeptide fragment, the 14-3-3 protein polypeptide fragment, and the bZIP transcription factor polypeptide fragment include the polypeptide fragments (i) to (iii) of the above-mentioned item 9, respectively.

12. A crystal of a florigen activation complex, including a florigen bound to a bZIP transcription factor via a 14-3-3 protein.
13. A crystal of a florigen activation complex according to the above-mentioned item 12, in which the crystal has a space group of P1, P6522, or P4 and lattice constants of a=74 to 158 Å, b=64 to 158 Å, c=96 to 500 Å, α=66 to 90°, β=85 to 90°, and γ=75 to 120°.
14. A method of producing the crystal of a florigen activation complex according to the above-mentioned item 12 or 13, the method including the steps of:

crystallizing a solution containing a complex of a florigen and a 14-3-3 protein by a vapor diffusion method using a precipitant solution containing at least as a precipitant 5 to 35 vol % polyethylene glycol;

collecting the resultant crystal; and

obtaining a crystal of a florigen activation complex by incubating the resultant crystal using a precipitant solution containing at least as a precipitant 5 to 35 vol % polyethylene glycol and containing a bZIP transcription factor.

15. A method of screening a substance that regulates flowering of a plant by designing and/or selecting a candidate substance having a function of regulating an activity of a florigen activation complex using a computer, the method including the steps of:
(a) causing storage means to store conformational information obtained from the crystal of a florigen activation complex of the above-mentioned item 12 or 13;
(b) causing deriving means to derive a three-dimensional conformation model based on the conformational information;
(c) causing calculation means to calculate an interatomic distance in the derived three-dimensional conformation model; and
(d) causing calculation means to calculate, based on the calculated interatomic distance, conformational information on a candidate substance capable of enhancing and/or inhibiting binding between a florigen and a 14-3-3 protein and/or binding between a 14-3-3 protein and a bZIP transcription factor, and to design and/or select the candidate substance.
16. A transformant, in which binding between a florigen and a 14-3-3 protein and/or binding between a 14-3-3 protein and a bZIP transcription factor are/is promoted and/or suppressed, the transformant having at least one of the polynucleotide of the above-mentioned item 10 introduced therein so that the polynucleotide is capable of being expressed.
17. A crystal of a florigen activation complex according to the above-mentioned item 12 or 13,

in which:

the florigen includes a polypeptide including an amino acid sequence which includes at least a sequence of amino acids at positions 62 to 132, starts with one of amino acids at positions 1 to 6, and ends with one of amino acids at positions 165 to 177, in an amino acid sequence set forth in SEQ ID NO: 1;

the 14-3-3 protein includes a polypeptide including an amino acid sequence which includes at least a sequence of amino acids at positions 51 to 227, starts with one of amino acids at positions 1 to 5, and ends with one of amino acids at positions 230 to 256, in an amino acid sequence set forth in SEQ ID NO: 2; and

the bZIP transcription factor includes a polypeptide including an amino acid sequence which includes at least a sequence of amino acids at positions 189 to 195, starts with one of amino acids at positions 182 to 188, and ends with the amino acid at position 195, in an amino acid sequence set forth in SEQ ID NO: 3.

18. A crystal of a florigen activation complex according to any one of the above-mentioned items 12, 13, and 17, in which the florigen includes a polypeptide including a sequence of amino acids at positions 6 to 170 in the amino acid sequence set forth in SEQ ID NO: 1, the 14-3-3 protein includes a polypeptide including a sequence of amino acids at positions 1 to 235 in the amino acid sequence set forth in SEQ ID NO: 2, and the bZIP transcription factor includes a polypeptide including a sequence of amino acids at positions 187 to 195 in the amino acid sequence set forth in SEQ ID NO: 3.
19. A crystal of a florigen activation complex according to any one of the above-mentioned items 12, 13, 17, and 18, in which the crystal is selected from the following:
(1) a crystal having a space group of P1 and lattice constants of a=74 to 79 Å, b=94 to 99 Å, c=96 to 101 Å, α=66 to 70°, β=85 to 90°, and γ=75 to 79°;
(2) a crystal having a space group of P6522 and lattice constants of a=125 to 135 Å, b=125 to 135 Å, c=340 to 344 Å, α=90°, β=90°, and γ=120°; and
(3) a crystal having a space group of P4 and lattice constants of a=153 to 158 Å, b=153 to 158 Å, c=495 to 498 Å, α=90°, β=90°, and γ=90°.
20. A crystal of a florigen activation complex according to any one of the above-mentioned items 12, 13, and 17 to 19, in which the crystal is selected from the following:
(1) a crystal having a space group of P1 and lattice constants of a=76.7 Å, b=96.6 Å, c=99.5 Å, α=68.2°, β=87.9°, and γ=77.9°;
(2) a crystal having a space group of P1 and lattice constants of a=76.8 Å, b=97.3 Å, c=99.8 Å, α=68.1°, β=87.8°, and γ=77.9°;
(3) a crystal having a space group of P1 and lattice constants of a=76.2 Å, b=96.1 Å, c=99.1 Å, α=68.2°, β=88.6°, and γ=77.8°;
(4) a crystal having a space group of P6522 and lattice constants of a=129.0 Å, b=129.0 Å, c=342.0 Å, α=90°, β=90°, and γ=120°; and
(5) a crystal having a space group of P4 and lattice constants of a=155.9 Å, b=155.9 Å, c=496.4 Å, α=90°, β=90°, and γ=90°.
21. A method of producing a crystal of a florigen activation complex according to the above-mentioned item 14, in which the concentration of the solution containing a complex of a florigen and a 14-3-3 protein is 5 to 40 mg/mL, the precipitant solution in the step of crystallizing a solution containing a complex of a florigen and a 14-3-3 protein contains 0.05 to 0.1 M HEPES (pH 6.5 to 8.5), 0.01 to 0.4 M ammonium sulfate, and 15 to 30 vol % polyethylene glycol having a molecular weight of 2,000 to 4,000, and the precipitant solution in the step of obtaining a crystal of a florigen activation complex contains 15 to 30 vol % ethylene glycol, a 1 to 5 mM bZIP transcription factor, 0.05 to 0.1 M HEPES (pH 6.5 to 8.5), 0.2 to 1 M ammonium sulfate, and 15 to 30 vol % polyethylene glycol having a molecular weight 2,000 to 4,000.
22. A method of screening a substance that regulates flowering of a plant according to the above-mentioned item 15, in which the conformational information is one described in Table 1 or Table 2.
23. A method of screening a substance that regulates flowering of a plant according to the above-mentioned item 15 or 22, in which a binding site between the florigen and the 14-3-3 protein includes at least one site selected from the group consisting of D62, M63, R64, P96, T98, F103, and R132 in SEQ ID NO: 1 and F200, D201, I204, E212, Y215, R226, and D227 in SEQ ID NO: 2, and a binding site between the 14-3-3 protein and the bZIP transcription factor includes at least one site selected from the group consisting of K51, R58, F121, R131, L224, and D227 in SEQ ID NO: 2 and R189 to F195 in SEQ ID NO: 3.
24. A method of screening a substance that regulates flowering of a plant according to any one of the above-mentioned items 15, 22, and 23, the method further including the steps of: acquiring the designed or selected candidate substance; and bringing the candidate substance into contact with a florigen, a 14-3-3 protein, and/or a bZIP transcription factor for investigating a function of regulating an activity of a florigen activation complex of the candidate substance.
25. A method of screening a substance that regulates flowering of a plant according to any one of the above-mentioned items 15 and 22 to 24, the method further including the steps of: producing a crystal of a florigen activation complex by the method of producing a crystal of a florigen activation complex according to the above-mentioned item 14 or 21; and obtaining conformational information on the florigen activation complex by subjecting the crystal to X-ray crystallographic analysis.

### Advantageous Effects of Invention

The crystal of the present invention provides conformational information on a florigen activation complex important for the elucidation of a mechanism for regulating flowering of plants. In addition, the crystal may be used as a material for additional research on the mechanism for regulating flowering. Further, it is possible to artificially regulate flowering of plants based on the conformational information obtained from the crystal of the present invention. In the present invention, the binding site of each protein in the florigen activation complex has been clarified. Information on the binding site may be used as a novel target of floral regulation, which can contribute to efficient floral regulation. A transgenic plant, which may be widely utilized for an increase of yield of an agricultural product, an improvement in efficiency of breeding, and the like, can be obtained based on the conformational information obtained in the present invention.

### BRIEF DESCRIPTION OF DRAWINGS

FIG. 1 A view illustrating a conformation of a florigen activation complex including three proteins Hd3a, GF14c, and OsFD1.

FIG. 2 An image showing the results of in vitro GST pull-down assays of three combinations, i.e., Hd3a and OsFD1, Hd3a and GF14c, GF14c and OsFD1 (Example 1(1)).

FIG. 3 A graph showing the results of analysis of an interaction between Hd3a and GF14c using NMR (Example 1(2)).

FIG. 4 Views illustrating the details of an interaction between Hd3a and GF14c. FIG. 4b is an expanded view illustrating the vicinity of Hd3a R64 (Example 3).

FIG. 5 Images showing the results of an in vitro GST pull-down assay with GF14c using mutant Hd3a (FIG. 5a) and the results of an in vitro GST pull-down assay with Hd3a using mutant GF14c (FIG. 5b) (Example 6).

FIG. 6 An image showing the results of a gel-shift assay of a florigen activation complex including Hd3a, GF14c, and OsFD1 and C-box DNA (Example 7).

FIG. 7 An image showing the results of confirmation of an interaction between each isoform of OsGF14 and Hd3a by a yeast two-hybrid method (Example 8).

FIG. 8 Images showing the results of confirmation of interactions among Hd3a, OsGF14b, and OsFD1 by a yeast two-hybrid method (Example 9).

FIG. 9 An image showing the results of confirmation of interactions among Hd3a, OsGF14b, and OsFD1 in the shoot apex of rice by immunoprecipitation (Example 10).

FIG. 10 An image showing the results of confirmation of subcellular localization of Hd3a, OsGF14b, and OsFD1 (Example 11).

FIG. 11 Images showing the results of confirmation of subcellular localization of Hd3a, OsGF14b, and OsFD1 (Example 11).

FIG. 12 Images showing the results of confirmation of subcellular localization of Hd3a, OsGF14b, and OsFD1 (Example 11).

FIG. 13 Graphs showing the results of confirmation of an influence of transient expression of Hd3a, OsGF14b, and OsFD1 on OsMADS15 gene transcription (Example 12).

FIG. 14 An image showing the results of confirmation of an influence of Hd3a mutation on flowering of plants (Example 13-1).

FIG. 15 A view illustrating an action mechanism of a florigen activation complex, which is obtained by the present invention.

FIG. 16 An image showing the results of confirmation of an influence of Hd3a mutation on flowering of plants (Example 13-2).

### DESCRIPTION OF EMBODIMENTS

In general, a plant has a vegetative growth stage and a reproductive growth stage and forms flower buds upon the transition of a growth phase from vegetative growth to reproductive growth. This phenomenon of transition from vegetative growth to reproductive growth is referred to as “flowering.” For example, the flowering in rice and wheats as monocotyledonous plants is heading. The heading refers to that the internode (panicle base) below the ear rapidly elongates and appears from the leaf sheath of the flag leaf. The time when the ear tip appears and the time when the ear including the base portion completely appears are defined as the heading in rice and wheats, respectively.

In the present invention, the plant means a higher plant having a florigen, and is preferably a short-day plant, which forms flower buds when sunshine duration per day is equal to or less than a predetermined time period, more preferably a monocotyledonous short-day plant, still more preferably a gramineous plant, most preferably rice.

(Florigen Activation Complex)

The present invention relates to a florigen activation complex including a complex of a florigen, a 14-3-3 protein, and a bZIP transcription factor, in which the florigen is bound to the bZIP transcription factor via the 14-3-3 protein.

In the present invention, the florigen is not particularly limited, and examples thereof include rice Hd3a and RFT1 (Genbank Accession No. AB062676), Arabidopsis FT (Genbank Accession No. AB027504) and TSF (Genbank Accession No. AB027506), tomato SFT (Genbank Accession No. AY186735), and wheat VRN3 (Genbank Accession No. LOC100037541). Of those, rice Hd3a is preferred. Rice Hd3a includes a protein represented by an amino acid sequence set forth in SEQ ID NO: 1 (Os06g0157700) (Genbank Accession No. BAB61028: derived from Oryza sativa Japonica group cultivar Nipponbare), or a protein which is formed of an amino acid sequence having deletions, substitutions, additions, and/or insertions of one to several amino acid residues in the amino acid sequence set forth in SEQ ID NO: 1, and functions as a florigen. Rice Hd3a in the present invention is preferably a florigen polypeptide fragment which includes at least a sequence of amino acids at positions 62 to 132, starts with one of amino acids at positions 1 to 6, and ends with one of amino acids at positions 165 to 177 in SEQ ID NO: 1, more preferably a polypeptide fragment formed of a sequence of amino acids at positions 6 to 170 in SEQ ID NO: 1. Further, it is particularly preferred that the polypeptide fragment have mutations of C43L/C109S/C166S in SEQ ID NO: 1.

In the present invention, the 14-3-3 protein is not particularly limited but is preferably rice GF14. Rice GF14 has eight isoforms, i.e., OsGF14a (Os08g0480800), OsGF14b (Os04g0462500), OsGF14c (Os08g0430500), OsGF14d (Os11g0546900), OsGF14e (Os02g0580300), OsGF14f (Os03g0710800), OsGF14g (Os01g0209200), and OsGF14h (Os11g0609600). In the present invention, GF14b, GF14c, GF14e, or GF14f is preferred, and GF14c or GF14b is more preferred. It should be noted that, in this description, numbers beginning with Os are numbers of gene loci specified in the Rice annotation project database (RAP-DB), unless otherwise stated. Those numbers may be used for specifying amino acid sequences and base sequences of proteins such as GF14 isoforms.

Rice GF14c includes a protein represented by an amino acid sequence set forth in SEQ ID NO: 2 (Os08g0430500) (Genbank Accession No. AAB07457.1: derived from Oryza sativa Japonica group cultivar Nipponbare), or a protein which is formed of an amino acid sequence having deletions, substitutions, additions, and/or insertions of one to several amino acid residues in the amino acid sequence set forth in SEQ ID NO: 2, and functions as a 14-3-3 protein to bind to a florigen. The 14-3-3 protein in the present invention is preferably a polypeptide fragment which includes at least a sequence of amino acids at positions 51 to 227, starts with one of amino acids at positions 1 to 5, and ends with one of amino acids at positions 230 to 256 in SEQ ID NO: 2, more preferably a polypeptide fragment formed of a sequence of amino acids at positions 1 to 235 in SEQ ID NO: 2.

Further, rice GF14b includes a protein represented by an amino acid sequence set forth in SEQ ID NO: 4 (Os04g0462500) (Genbank Accession No. AK071822: derived from Oryza sativa Japonica group cultivar Nipponbare), or a protein which is formed of an amino acid sequence having deletions, substitutions, additions, and/or insertions of one to several amino acid residues in the amino acid sequence set forth in SEQ ID NO: 4, and functions as a 14-3-3 protein to bind to a florigen. Rice GF14b has an amino acid sequence shifted by 6 residues as compared to the amino acid sequence of GF14c. The sequence of amino acids at positions 51 to 227 in SEQ ID NO: 2 corresponds to a sequence of amino acids at positions 57 to 233 in SEQ ID NO: 4. The amino acid sequence which starts with one of amino acids at positions 1 to 5 and ends with one of amino acids at positions 230 to 256 in SEQ ID NO: 2 corresponds to an amino acid sequence which starts with one of amino acids at positions 7 to 11 and ends with one of amino acids at positions 236 to 262 in SEQ ID NO: 4.

In the present invention, the bZIP transcription factor is not particularly limited and examples thereof include rice OsFD1 and Arabidopsis FD (Genbank Accession No. AB105823). Of those, rice OsFD1 is preferred. Rice OsFD1 includes a protein represented by an amino acid sequence set forth in SEQ ID NO: 3 (Os09g0540800: derived from Oryza sativa Japonica group cultivar Nipponbare) (Rice genome annotation locus No. LOC_Os09g36910.1 specified in the TIGR Rice Genome Annotation Database), or a protein which is formed of an amino acid sequence having deletions, substitutions, additions, and/or insertions of one to several amino acid residues in the amino acid sequence set forth in SEQ ID NO: 3, and functions as a bZIP transcription factor. The bZIP transcription factor in the present invention is preferably a polypeptide fragment which includes at least a sequence of amino acids at positions 189 to 195, starts with one of amino acids at positions 182 to 188, and ends with the amino acid at position 195 in SEQ ID NO: 3, more preferably a polypeptide fragment formed of a sequence of amino acids at positions 187 to 195 in SEQ ID NO: 3. Further, in the polypeptide fragment, serine at position 192 is preferably phosphorylated. The phosphorylation of the serine is considered to be necessary for the binding of the 14-3-3 protein to the bZIP transcription factor.

The florigen, the 14-3-3 protein, and the bZIP transcription factor bind to each other to form a florigen activation complex. A region of a sequence of amino acids at positions 62 to 132 in SEQ ID NO: 1 or a region corresponding to the region in the florigen having an amino acid sequence other than SEQ ID NO: 1, and/or a region of a sequence of amino acids at positions 200 to 227 in SEQ ID NO: 2 or a region corresponding to the region in the 14-3-3 protein having an amino acid sequence other than SEQ ID NO: 2 are/is considered to be important for binding between the florigen and the 14-3-3 protein. Further, it has been clarified in the present invention that a region of a sequence of amino acids at positions 51 to 227 in SEQ ID NO: 2 or a region corresponding to the region in the 14-3-3 protein having an amino acid sequence other than SEQ ID NO: 2, and/or a region of a sequence of amino acids at positions 189 to 195 in SEQ ID NO: 3 or a region corresponding to the region in the bZIP transcription factor having an amino acid sequence other than SEQ ID NO: 3 are/is important for binding between the 14-3-3 protein and the bZIP transcription factor.

In addition, among those regions, amino acids of D62, M63, R64, P96, T98, F103, and R132 in SEQ ID NO: 1 or amino acids corresponding to the amino acids in the florigen having an amino acid sequence other than SEQ ID NO: 1, and/or amino acids of F200, D201, I204, E212, Y215, R226, and D227 in SEQ ID NO: 2 or amino acids corresponding to the amino acids in the 14-3-3 protein having an amino acid sequence other than SEQ ID NO: 2 are considered to be important for binding between the florigen and the 14-3-3 protein. Further, it has been clarified in the present invention that amino acids of K51, R58, F121, R131, L224, and D227 in SEQ ID NO: 2 or amino acids corresponding to the amino acids in the 14-3-3 protein having an amino acid sequence other than SEQ ID NO: 2 are important for binding between the 14-3-3 protein and the bZIP transcription factor.

The “site of a protein corresponding to a particular amino acid (e.g., D62) in SEQ ID NO: X” to be used in the present invention means to include, in addition to a site of a particular amino acid (e.g., D62) in SEQ ID NO: X, a site corresponding to the particular amino acid (e.g., D62) in a protein having an amino acid sequence other than SEQ ID NO: X and having a function equivalent to that of a protein of SEQ ID NO: X. For example, the “sites of the 14-3-3 protein corresponding to F200, D201, I204, E212, Y215, and R226 in SEQ ID NO: 2” include, in addition to sites of F200, D201, I204, E212, Y215, and R226 in SEQ ID NO: 2, amino acids corresponding to the amino acids in the 14-3-3 protein having an amino acid sequence other than SEQ ID NO: 2, for example, F206, D207, I210, E218, Y221, and R232 of rice GF14b (corresponding to F200, D201, I204, E212, Y215, and R226 of rice GF14c).

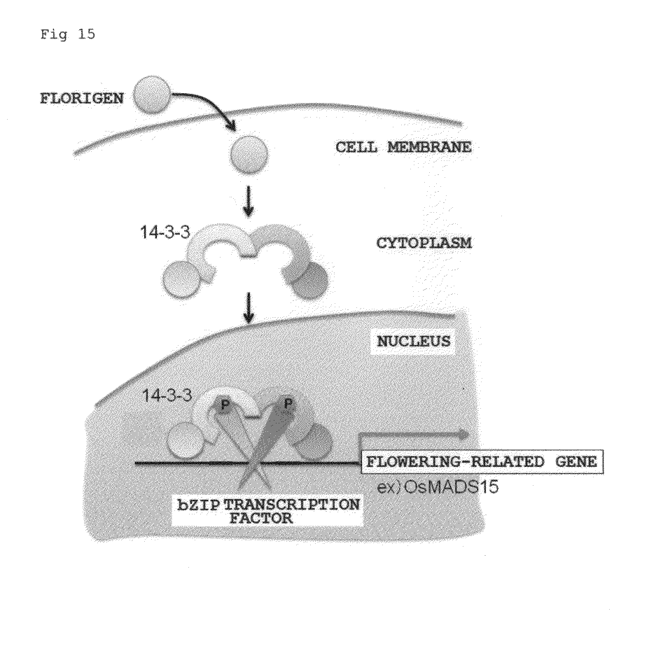

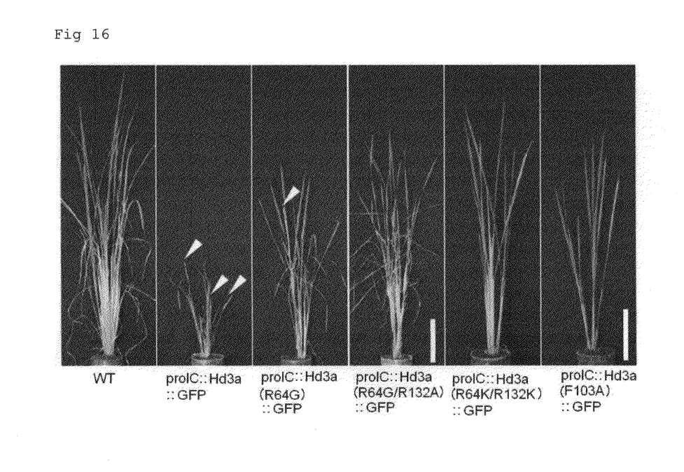

The action mechanism of a florigen activation complex in flowering is described (see FIG. 15). A 14-3-3 protein acts as an adapter of a florigen transported from leaves to the shoot apex. When the florigen enters shoot apex cells, the florigen is considered to first bind to the 14-3-3 protein in the cytoplasm. At this stage, OsFD1 has been expressed in the shoot apex cells, and such complex of the florigen and the 14-3-3 protein enters the nucleus from the cytoplasm and binds to a bZIP transcription factor to form a florigen activation complex, which is retained in the nucleus. When the series of processes are initiated, the florigen activation complex is accumulated in the nucleus. After that, the florigen activation complex activates the transcription of a flowering-related gene (e.g., an OsMADS15 gene), which causes floral induction. In such action mechanism, the florigen is trapped by the 14-3-3 protein in the cytoplasm of the shoot apex cells, and the complex of the florigen and the 14-3-3 protein is efficiently transported by the bZIP transcription factor into the nucleus for the transcriptional activation of a gene necessary for floral induction. Further, the transcriptional activation of the flowering-related gene by the florigen activation complex in the present invention is considered to occur through the binding of the florigen activation complex to C-box DNA. C-box DNA is represented by a base sequence of GACGTC (SEQ ID NO: 10) and is present in a promoter region of the flowering-related gene. The florigen activation complex is estimated to form a stable complex on the C-box of the flowering-related gene and activate the transcription of the flowering-related gene.

In the present invention, the florigen, the 14-3-3 protein, the bZIP transcription factor, mutant proteins thereof, a complex of the florigen and the 14-3-3 protein, a complex of the 14-3-3 protein and the bZIP transcription factor, and the florigen activation complex as a complex of the three proteins may be in such a state that they are isolated and purified from cells, tissues, and the like, or may be in such a state that a gene encoding a protein is introduced into host cells such as yeast and Escherichia coli and the protein is expressed in the cells. Further, the present invention also encompasses polynucleotides encoding those proteins. To those proteins, depending on expression systems of host cells, a peptide fragment may be added in such an amount that functions of the proteins are not affected. For example, in the case of using an expression system of Escherichia coli, an Escherichia coli expression plasmid-derived peptide fragment (GPGHM) has been added to the N-terminal of a protein.

The complex of the florigen and the 14-3-3 protein or the complex of the 14-3-3 protein and the bZIP transcription factor may be produced by bringing the two proteins into contact with each other in an isolated and purified state or in a living body. Similarly, the florigen activation complex as a complex of the three proteins may also be produced by bringing the three kinds of proteins or any one of the complexes of two proteins and the remaining protein into contact with each other in an isolated and purified state or in a living body such as cells.

(Production Method for Crystal of Florigen Activation Complex or Florigen)

First, a protein solution is produced. A protein is allowed to be present in a solution formed of a buffer, a salt, a reducing agent, and the like. Any buffer, salt, and reducing agent may be used as long as the conformation of the protein is not affected. Examples of the buffer include 1 to 500 mM Na-HEPES, sodium phosphate, potassium phosphate, and Tris-HCl. Examples of the salt include 1 mM to 1 M sodium chloride, lithium chloride, and magnesium chloride. Examples of the reducing agent include 0.1 to 10 mM β-mercaptoethanol and dithiothreitol (DTT). Further, the protein solution may contain dimethylsulfoxide (DMSO) or ethylene glycol. The solution containing the protein has a pH of 4 to 11, preferably a pH of 6 to 9. Such protein solution or florigen solution may be used for crystallization without any further treatment, or as necessary, a preservative, a stabilizer, a surfactant, or the like is further added to the solution, and the resultant solution may be used for crystallization.

As a method of crystallizing a protein (polypeptide), a general technique for protein crystallization such as a vapor diffusion method, a batch method, or a dialysis method may be employed. Further, in the crystallization of a protein, it is important to determine physical and chemical factors such as the concentration of the protein, the concentration of a salt, a pH, the kind of a precipitant, and a temperature.

The vapor diffusion method refers to a method involving placing a droplet of a protein solution including a precipitant in a container including a buffer (external solution) containing the precipitant at a higher concentration, sealing the container, and then leaving the resultant to stand still. The vapor diffusion method is classified into a hanging drop method and a sitting drop method depending on how to place the droplet, and any of the methods may be adopted in the present invention. The hanging drop method is a method involving placing a small droplet of a protein solution on a cover glass, inverting the cover glass in a reservoir, and sealing the reservoir. On the other hand, the sitting drop method is a method involving installing an appropriate droplet stage in a reservoir, placing a droplet of a protein solution on the droplet stage, and sealing the reservoir with a cover glass or the like. In any of the methods, a precipitant is incorporated into the solution in the reservoir (reservoir solution). As appropriate, a small amount of the precipitant may be incorporated into a protein small droplet.

The reservoir solution (also referred to as precipitant solution) to be used in the vapor diffusion method is a solution formed of a buffer, a precipitant, a salt, and the like. Any buffer, precipitant, and salt may be used as long as a crystal can be efficiently produced. For example, the buffer is selected from 5 to 200 mM Na-HEPES, sodium phosphate, potassium phosphate, Tris-HCl, sodium acetate, citric acid, cacodylic acid, and the like at a pH of 4 to 10, the precipitant is selected from 5 to 35 vol % polyethylene glycol (PEG) having a molecular weight of 550 to 20,000, 0.2 to 2 M ammonium sulfate, 5 to 35 vol % methylpentanediol (MPD), 0.2 to 2 M ammonium tartrate, 5 to 35 vol % isopropanol, and a combination thereof, and the salt is selected from 0.2 to 4 M sodium chloride, lithium chloride, magnesium chloride, and the like. The components for the reservoir solution are not limited to those described above.

A crystal of a florigen activation complex of the present invention may be produced as described below. A florigen and a 14-3-3 protein are mixed with each other and dialyzed against a 1 to 50 mM Tris-HCl buffer (pH 6.5 to 8.5) containing 10 to 50 mM NaCl to prepare a sample of a complex. The protein solution (protein concentration: 5 to 40 mg/mL) is mixed with a precipitant solution (0.05 to 0.1 M HEPES (pH 6.5 to 8.5), 0.01 to 0.4 M ammonium sulfate, and 15 to 30 vol % PEG (molecular weight: 2,000 to 4,000)), and the crystallization of a complex of the florigen and the 14-3-3 protein is performed by a sitting drop method under the condition of 4 to 20° C. After about 1 to 3 weeks, a single crystal of the complex of the florigen and the 14-3-3 protein is obtained. The obtained single crystal is collected and incubated with a precipitant solution (0.05 to 0.1 M HEPES (pH 6.5 to 8.5), 0.01 to 0.4 M (preferably 0.15 to 0.25 M) ammonium sulfate, 15 to 30 vol % (preferably 23 to 27 vol %) PEG (molecular weight: 2,000 to 4,000)) containing 15 to 30 vol % ethylene glycol and a 1 to 5 mM bZIP transcription factor for 10 to 20 minutes. Thus, a crystal of a florigen activation complex suitable for X-ray crystallographic analysis can be obtained.

The crystal of a florigen activation complex of the present invention may be preferably produced as described below. A florigen and a 14-3-3 protein are mixed with each other at a molar ratio of 1:1 to 2 (more preferably 1:1.5) and dialyzed against a 10 mM Tris-HCl buffer (pH 7.5) containing 20 mM NaCl to prepare a sample of a complex. 1 μl of the protein solution (protein concentration: 10 mg/mL) is mixed with 1 μl of a precipitant solution (0.1 M HEPES (pH 7.5), 0.2M ammonium sulfate, and 25 vol % PEG 3350), and the crystallization of a complex of the florigen and the 14-3-3 protein is performed by a sitting drop method under the condition of 4° C. After about 1 to 3 weeks, a single crystal of the complex of the florigen and the 14-3-3 protein is obtained. The obtained single crystal is collected and incubated with a precipitant solution ((0.1 M HEPES (pH 7.5), 0.2 M ammonium sulfate, 25 vol % PEG 3350) containing 25% ethylene glycol and a 2 mM bZIP transcription factor for 10 to 20 minutes (more preferably for 15 minutes). Thus, a crystal of a florigen activation complex can be obtained.

A crystal of a florigen in the present invention may be produced as described below. A purified florigen is dialyzed against a 1 to 50 mM Tris buffer (pH 5.5 to 7.5) containing 10 to 50 mM NaCl and concentrated so as to achieve a concentration of 5 to 40 mg/mL. The resultant protein solution is mixed with a precipitant solution (0.01 to 0.5 M (preferably 0.05 to 0.15 M) cacodylic acid (pH 5.5 to 7.5), 0.01 to 0.5 M (preferably 0.15 to 0.25 M) ammonium tartrate, and 10 to 50 vol % (preferably 25 to 35 vol %) PEG (molecular weight: 5,000 to 12,000), and crystallization is performed by a sitting drop method under the condition of 4 to 20° C. After about 0.5 to 3 days (preferably about 1 day), a crystal of a florigen activation complex suitable for X-ray crystallographic analysis can be obtained.

The crystal of a florigen in the present invention may be preferably produced as described below. A purified florigen is dialyzed against a 10 mM Tris buffer (pH 7.5) containing 20 mM NaCl and concentrated so as to achieve a concentration of 5 mg/ml. 1 μl of the resultant protein solution is mixed with 1 μl of a precipitant solution (0.1 M cacodylic acid (pH 6.5), 0.2 M ammonium tartrate, and 30 vol % PEG 8000), and crystallization is performed by a sitting drop method under the condition of 4° C. After about 0.5 to 3 days (preferably about 1 day), a crystal of a florigen activation complex suitable for X-ray crystallographic analysis can be obtained.

In the present invention, it is preferred to obtain a crystal having such quality as to provide at least a resolution of 10 Å or less, preferably a resolution of 4.0 Å or less, more preferably a resolution of 3.4 Å or less, still more preferably a resolution of 2.8 Å or less, particularly preferably a resolution of 2.4 Å or less when the crystal is subjected to X-ray crystallographic analysis (“Introduction to Protein Structure” Carl Brandon & John Tooze, translated by Yukiteru Katsube et al., Kyoikusha, 1992, pp. 276-277).

(Crystal of Florigen Activation Complex or Florigen)

The crystal of a florigen activation complex or a florigen of the present invention is substantially free of impurities, and has an activity even when dissolved again. Examples of the impurities include a decomposition product of GST, a florigen, or a florigen activation complex and a protein peculiar to Escherichia coli.

The crystal of a florigen activation complex of the present invention has a space group of P1, P6522, or P4 and lattice constants of a=74 to 158 Å, b=64 to 158 Å, c=96 to 500 Å, α=66 to 90°, β=85 to 90°, and γ=75 to 120°. The crystal obtained by the present invention has sufficient quality and size to conduct X-ray crystallographic analysis at a resolution of about 1.0 Å to about 3.5 Å.

The crystal of a florigen activation complex of the present invention is preferably selected from the following crystals:

(1) a crystal having a space group of P1 and lattice constants of a=74 to 79 Å, b=94 to 99 Å, c=96 to 101 Å, α=66 to 70°, β=85 to 90°, and γ=75 to 79°;
(2) a crystal having a space group of P6522 and lattice constants of a=125 to 135 Å, b=125 to 135 Å, c=340 to 344 Å, α=90°, β=90°, and γ=120°; and
(3) a crystal having a space group of P4 and lattice constants of a=153 to 158 Å, b=153 to 158 Å, c=495 to 498 Å, α=90°, β=90°, and γ=90°.

The crystal of a florigen activation complex of the present invention is more preferably selected from the following crystals:

(florigen activation complex 1) a crystal having a space group of P1 and lattice constants of a=76.7 Å, b=96.6 Å, c=99.5 Å, α=68.2°, β=87.9°, and γ=77.9° at a resolution of 2.4 Å;
(florigen activation complex 2) a crystal having a space group of P1 and lattice constants of a=76.8 Å, b=97.3 Å, c=99.8 Å, α=68.1°, β=87.8°, and γ=77.9° at a resolution of 2.2 Å;
(florigen activation complex 3) a crystal having a space group of P1 and lattice constants of a=76.2 Å, b=96.1 Å, c=99.1 Å, α=68.2°, β=88.6°, and γ=77.8° at a resolution of 2.8 Å;
(florigen activation complex 4) a crystal having a space group of P6522 and lattice constants of a=129.0 Å, b=129.0 Å, c=342.0 Å, α=90°, β=90°, and γ=120° at a resolution of 2.85 Å; and
(florigen activation complex 5) a crystal having a space group of P4 and lattice constants of a=155.9 Å, b=155.9 Å, c=496.4 Å, α=90°, β=90°, and γ=90° at a resolution of 2.96 Å.

The crystal of a florigen of the present invention has a space group of P63 and lattice constants of a=65 to 67 Å, b=65 to 67 Å, c=58 to 61 Å, α=90°, β=90°, and γ=120°. The crystal obtained by the present invention has sufficient quality and size to conduct X-ray crystallographic analysis at a resolution of about 1.0 Å to about 3.5 Å (preferably 1.0 to 1.5 Å).

The crystal of a florigen of the present invention is preferably selected from the following crystals:

(florigen 1) a crystal having a space group of P63 and lattice constants of a=65.9 Å, b=65.9 Å, c=59.8 Å, α=90°, β=90°, and γ=120° at a resolution of 1.3 Å; and
(florigen 2) a crystal having a space group of P6522 and lattice constants of a=66.0 Å, b=66.0 Å, c=60.2 Å, α=90°, β=90°, and γ=120° at a resolution of 1.4 Å.

Each of those crystals of a florigen are excellent in resolution, and conformational information obtained by subjecting each of those crystals to X-ray structure analysis is suitable for use in docking simulation with a computer or the like.

(X-Ray Crystallographic Analysis)

X-ray crystallographic analysis is most commonly performed as a technique for clarifying a conformation of a protein (polypeptide). This technique involves crystallizing a protein, irradiating the crystal with a monochromatic X-ray, and clarifying conformational information on the protein based on the resultant X-ray diffraction image. The conformational information includes an electron density map and atomic coordinates, and the atomic coordinates may be acquired by analysis according to a method known in the art (D. E. McRee, Practical Protein Crystallography, Academic Press, San Diego (1993)).

The X-ray crystallographic analysis involves the steps of: irradiating a crystal with an X-ray to acquire diffraction data; analyzing the resultant diffraction data to acquire an electron density of a protein (polypeptide); and analyzing the resultant electron density to acquire atomic coordinates of the protein (polypeptide).

In the X-ray crystallographic analysis, through the use of an X-ray diffractometer in a laboratory or a large radiation facility (e.g., ESRF, APS, SPring-8, PF, ALS, CHESS, SRS, LLNL, SSRL, or Brookhaven), diffraction data is collected by oscillation photography or the like with a two-dimensional detector such as an imaging plate or a CCD camera, and an electron density may be obtained from the data to elucidate atomic coordinates.

A crystal of a protein often undergoes damage by irradiation with an X-ray, resulting in a deterioration in diffraction ability. Hence, it is preferred to perform high-resolution X-ray diffraction through low-temperature measurement. The low-temperature measurement refers to a method involving freezing a crystal by rapidly cooling to about −173° C., and collecting diffraction data in the state. In general, in the freezing of a crystal of a protein, a contrivance such as treatment in a solution containing a protectant (cryoprotectant) such as glycerol is made for the purpose of preventing the collapse of the crystal due to the freezing. A frozen crystal may be prepared, for example, by flash freezing involving directly immersing a crystal, which has been immersed in a preservative solution supplemented with a protectant, in liquid nitrogen.

A diffraction image collected by an X-ray diffraction experiment may be processed with data processing software to calculate diffraction intensities obtained by the indexing and integration of individual diffraction spots. Electron densities in a three-dimensional space are derived by performing inverse Fourier transform using the diffraction intensities and phase information of the diffraction spots. In a diffraction experiment, it is impossible in principle to measure phase information on each of the diffraction spots necessary for the calculation of the electron density. Hence, in order to obtain the electron density, the phase as lost information is estimated by a molecular replacement method, a heavy atom isomorphous replacement method, a multiwavelength anomalous dispersion method (MAD method), or a modified method thereof.

An electron density map is depicted based on the thus obtained electron density, and a three-dimensional model is constructed using software which operates in a graphics workstation in accordance with the electron density map. After the construction of the model, structural refinement is performed by a least-squares method or the like to give final atomic coordinates (conformational coordinates) of a protein.

(Conformational Information on Florigen Activation Complex or Florigen)

The atomic coordinates mean mathematical coordinates in which the positions of the atoms of the protein described above are expressed as three-dimensional coordinates. The atomic coordinates substantially mean a space configuration determined depending on distances between the respective molecules (atoms) which construct a chemical structure. When the space configuration is processed on a computer as information, a relative configuration is converted into numerical information as specific coordinates in a certain coordinate system (referred to as conversion to coordinates). This is processing necessary for convenience in performing computer processing, and it should be understood that the nature of the atomic coordinates is a configuration determined depending on distances between the respective molecules (atoms) as described above and is not coordinate values specified temporarily at the time of computer processing. Further, the atomic coordinates as used herein mean coordinates of individual atoms which construct a substance (such as a protein or an amino acid).

In this description, Table 1 shows atomic coordinates of the florigen activation complex 1, and Table 2 shows atomic coordinates of the florigen 1. The data of Tables 1 and 2 are described in conformity with the format of the protein data bank (PDB) (http://www.wwpdb.org/documentation/format23/v2.3.html). Further, in the present invention, through the use of the conformational information represented by the electron density and atomic coordinates, atomic coordinates obtained by homology modeling or the like on a computer may also be obtained as derivatives for a protein having 40% or more homology to the polypeptide of the present invention.

(Method of Screening Substance that Regulates Flowering of Plant)

In a florigen activation complex including a florigen, a 14-3-3 protein, and a bZIP transcription factor, a substance capable of regulating flowering may be obtained from numerous substances by selecting a substance (including a compound) capable of affecting the binding of each protein and a substance (including a compound) having a structure capable of competitively binding to a binding site.

As one aspect of the screening method of the present invention, there is given a method of screening a substance that regulates flowering of a plant by designing and/or selecting a candidate substance having a florigen activation complex activity regulating function using a computer (hereinafter, also simply referred to as “screening method using a computer”). Such screening method involves the following steps of:

(a) causing storage means to store conformational information obtained from the florigen activation complex or crystal of a florigen of the present invention;
(b) causing deriving means to derive a three-dimensional conformation model based on the conformational information;
(c) causing calculation means to calculate an interatomic distance in the derived three-dimensional conformation model; and
(d) causing calculation means to calculate, based on the calculated interatomic distance, conformational information on a candidate substance capable of enhancing and/or inhibiting binding between a florigen and a 14-3-3 protein and/or binding between a 14-3-3 protein and a bZIP transcription factor, and to design and/or select the candidate substance.

Atomic coordinates of a binding site of each protein may be used as the conformational information on the florigen activation complex or the like, and the whole atomic coordinates of each protein, derivatives thereof including the binding site, and parts thereof may be utilized. Further, atomic coordinates of a binding site appropriately altered on a computer so as to become suitable for screening may be utilized in the present invention.

In the step (a) and the step (b), modes of three-dimensional chemical interactions among various proteins may be displayed in detail by inputting atomic coordinates out of the conformational information on the florigen activation complex or the florigen to a computer or a storage medium of the computer in which a computer program that displays atomic coordinates of a molecule operates. There are known a large number of commercially available computer programs that display atomic coordinates of a molecule. In general, those programs include means for inputting atomic coordinates of a molecule, means for deriving a three-dimensional conformation model based on conformational information by deriving means and visually displaying the coordinates on a computer screen, means for measuring or calculating a distance, a bond angle, and the like between atoms in the displayed molecule, means for additionally correcting the coordinates, and the like. In addition, it is also possible to use a program including means for calculating structural energy of a molecule based on coordinates of the molecule and means for calculating free energy in consideration of a solvent molecule such as a water molecule. Computer programs InsightII and QUANTA commercially available from Accerlys are suitably used for the screening method of the present invention. However, computer programs to be used in the present invention are not limited to the above-mentioned programs.

The candidate substance may be any of known and novel substances, and structures, origins, physical properties, and the like thereof are not particularly limited. Further, the candidate substance may be any of a natural compound, a synthetic compound, a high-molecular-weight compound, a low-molecular-weight compound, a peptide, and a nucleic acid analog. A known program has only to be used for the conversion of the conformation of the candidate substance into coordinates. For example, as a program that converts the conformation of the low-molecular-weight compound into coordinates, CORINA (http://www2.chemie.uni-erlangen.de/software/corina/index.html), Concord (http://www.tripos.com/sciTech/in SilicoDisc/chemInfo/concord.html), Converter, or the like may be utilized.

The steps (c) and (d) include the stage of evaluating the matching state of the atomic coordinates of the candidate substance and atomic coordinates having binding sites of a florigen, a 14-3-3 protein, and a bZIP transcription factor (or a complex of two out of the proteins or a florigen activation complex) by overlapping both the coordinates in the same coordinate system, or the stage of calculating an interatomic distance based on the atomic coordinates of the florigen activation complex or the like to design a candidate substance based on the interatomic distance. Those stages may be performed using the above-mentioned commercially available package software and a computer system capable of operating the software. The computer system appropriately includes various means necessary for operating software of interest, for example, storage means for storing a structural formula of a substance such as a compound, means for converting a conformation of a substance such as a compound into coordinates, storage means for storing atomic coordinates of a substance such as a compound, storage means for storing atomic coordinates of each protein to be used in the step (a), storage means for storing evaluation results, means for displaying contents in each storage means, input means such as a keyboard, display means such as a display, and a central processing unit.

Any software for analysis may be used as long as the software can perform an operation for docking a candidate substance to a protein on a computer, and for example, DOCK, FlexX (Tripos), LigandFit (Accelrys), Ludi (Accelrys), and the like may be used. In addition, the operation may be performed interactively using molecular display software such as InsightII. In that case, as an indicator in evaluating the matching state using each of those programs, a free energy calculated value for the whole complex, an empirical scoring function, shape complementarity evaluation, and the like may be arbitrarily selected and used. The indicator allows whether the binding is good or bad to be objectively evaluated.

The design or selection of a substance capable of regulating flowering using atomic coordinates of various proteins such as a florigen and a florigen activation complex allows quick screening on a computer. Further, it is desired to experimentally evaluate a group of candidate substances selected by screening utilizing a computer.

In the method of screening a substance that regulates flowering of a plant using a computer, in order to experimentally evaluate a function of regulating an action of a florigen activation complex of the candidate substance, the candidate substance is preferably synthesized or acquired. The candidate substance has only to be synthesized using a known technique or acquired, for example, by purifying a substance derived from a living body.

In addition, the resultant candidate substance is experimentally evaluated by subjecting the substance to, for example, a biochemical technique or a biological technique using various proteins such as a florigen, which makes it possible to select a more effective substance having a function of regulating an action of a florigen activation complex, and further, a substance that regulates flowering.

As another aspect of the screening method of the present invention, there is given a method itself of selecting a substance having a function of regulating an action of a florigen activation complex (i.e., a substance having a function capable of regulating flowering) using a biochemical technique or a biological technique (hereinafter, also simply referred to as “screening method using a biochemical technique or the like”). In order to confirm whether or not the candidate substance exhibits a function of regulating an action of a florigen activation complex, it is recommended to examine whether or not there is a difference in action of a florigen activation complex, e.g., whether or not there is a difference in amount of a florigen activation complex between the cases where the candidate substance is added and is not added to a system in which the function of regulating an action of a florigen activation complex can be confirmed. The system in which the function of regulating an action of a florigen activation complex can be confirmed is exemplified by the step of bringing the candidate substance into contact with a florigen, a 14-3-3 protein, and/or a bZIP transcription factor, more specifically the following steps:

(1) a step including bringing a candidate substance into contact with any one of a florigen and a 14-3-3 protein, and bringing the candidate substance into contact with any one of the 14-3-3 protein and the florigen, respectively; and
(2) a step including bringing a candidate substance into contact with any one of a 14-3-3 protein to which a florigen is bound or unbound and a bZIP transcription factor, and bringing the candidate substance into contact with any one of the bZIP transcription factor and the 14-3-3 protein to which a florigen is bound or unbound, respectively.

The function of regulating an action of a florigen activation complex preferably means a function of enhancing and/or suppressing the formation of a florigen activation complex. It is considered that the enhancement and/or suppression of the formation of a florigen activation complex allows flowering of a plant to be regulated, that is, the flowering to be accelerated and/or delayed.

The step of bringing a candidate substance into contact with a florigen, a 14-3-3 protein, and/or a bZIP transcription factor may be performed by performing a yeast two-hybrid method, a BiFC method, or the like in the presence or absence of the candidate substance to confirm an interaction, and the function of regulating an action of a florigen activation complex may be evaluated. Alternatively, the function of regulating an action of a florigen activation complex may also be evaluated by fixing a florigen and a bZIP transcription factor on a plate, adding a fluorescence-labeled 14-3-3 protein and a candidate substance, and measuring the fluorescence intensity of the plate. In addition, isothermal titration calorimetry (ITC), surface plasmon resonance (SPR), or the like may be employed for evaluating the function of regulating an action of a florigen activation complex.

In the method of selecting a substance having a function of regulating an action of a florigen activation complex (further, a substance that regulates flowering), it is preferred to use, as an indicator, binding in a binding site between a florigen and a 14-3-3 protein and/or a binding site between a 14-3-3 protein and a bZIP transcription factor. The use of the binding in each of those binding sites as an indicator means confirming the binding state of a binding site in a florigen activation complex. When binding is inhibited in the presence of a candidate substance as compared to the case in the absence of the candidate substance, the candidate substance may be selected as a substance having a function of suppressing the formation of a florigen activation complex. When binding is promoted, the candidate substance may be selected as a substance having a function of enhancing the formation of a florigen activation complex.

The binding state of a binding site in a complex may be confirmed by an NMR method, a fluorescence labeling method, or the like.

In the NMR method, for example, in the case of binding an unlabeled 14-3-3 protein and bZIP transcription factor to a stable isotope-labeled florigen in the presence of a candidate substance, when the NMR spectrum of the florigen changes in a binding site-specific manner as compared to the case in the absence of the candidate substance, it can be confirmed that the candidate substance affected binding in the binding site. When NMR signals from the florigen have been assigned for all residues (for example, when the florigen is Hd3a, which amino acid of Hd3a gives each NMR peak has been identified), an amino acid to which the candidate substance is bound can be specified.

In the fluorescence labeling method, for example, an amino acid in the vicinity of a binding site is mutated into a cysteine residue and subjected to a reaction with a fluorescence reagent which acts in a cysteine-specific manner to introduce a fluorescence label, and the resultant may be used. When the binding state changes depending on the presence or absence of a candidate substance, it is considered that the intensity of the introduced fluorescence also changes. Thus, the binding state of the binding site can be confirmed.

The NMR method may be performed by dissolving a florigen, a 14-3-3 protein, and a bZIP transcription factor in a solution and measuring the NMR spectrum of the solution. Any solution may be used for dissolving the florigen and the like as long as the NMR spectrum can be measured, and for example, a buffer containing dithiothreitol (DTT), potassium chloride (KCl), sodium chloride (NaCl), and deuterium oxide is used. As an NMR measurement method, homonuclear multidimensional NMR measurement, heteronuclear multidimensional NMR measurement, or the like is preferably employed. For example, the measurement may be performed by an NMR measurement method called 1H-15NHSQC. Such measurement is a technology known to a person skilled in the art. 1H-15NHSQC is a correlation spectrum of a hydrogen atom and a nitrogen atom in a peptide bond in a protein, that is, a 1H-15N correlation spectrum, and information on individual residues may be obtained from a 1H-15N signal attributed to a main chain. Such NMR measurement method allows the conformation analysis of a target high-molecular-weight substance such as a protein, and allows the interaction analysis of a protein.

In the screening method of the present invention, a screening method using a computer and a screening method using a biochemical technique or the like may be employed in combination.

In addition, the evaluation of a function of regulating flowering of a plant, that is, a function of accelerating and/or delaying of flowering, of a candidate substance may be examined by feeding the candidate substance to a plant body (e.g., the candidate substance is absorbed with water from the root, or a transformant is produced by introducing a gene or the like encoding the candidate substance).

(Method of Regulating Flowering of Plant)

The present invention also encompasses a method of regulating flowering of a plant. The regulation of flowering means accelerating and/or delaying flowering. In more detail, the present invention relates to a method of regulating flowering of a plant, the method including promoting (enhancing) and/or suppressing the formation of a florigen activation complex by affecting any one or a plurality of binding sites shown in the following items (1) and/or (2), that is, by regulating binding in the binding sites:

(1) binding sites between a florigen and a 14-3-3 protein: sites of the florigen corresponding to D62, M63, R64, P96, T98, F103, and R132 in SEQ ID NO: 1 and sites of the 14-3-3 protein corresponding to F200, D201, I204, E212, Y215, R226, and D227 in SEQ ID NO: 2; and
(2) binding sites between a 14-3-3 protein and a bZIP transcription factor: sites of the 14-3-3 protein corresponding to K51, R58, F121, R131, L224, and D227 in SEQ ID NO: 2 and sites of the bZIP transcription factor corresponding to R189 to F195 in SEQ ID NO: 3.

The promotion (enhancement) and/or suppression of the formation of a florigen activation complex is exemplified by the introduction of a mutation into a binding site between a florigen and a 14-3-3 protein and/or a binding site between a 14-3-3 protein and a bZIP transcription factor, and the feeding of a substance capable of promoting and/or suppressing binding between a florigen and a 14-3-3 protein and/or binding between a 14-3-3 protein and a bZIP transcription factor to a plant body.

Examples of the substance capable of promoting binding between a florigen and a 14-3-3 protein and/or binding between a 14-3-3 protein and a bZIP transcription factor include a gene itself encoding the florigen, the 14-3-3 protein, or the bZIP transcription factor. Examples of the gene encoding the florigen, the 14-3-3 protein, or the bZIP transcription factor include genes encoding rice Hd3a (SEQ ID NO: 11), rice GF14b (SEQ ID NO: 14), rice GF14c (SEQ ID NO: 12), and rice FD1 (SEQ ID NO: 13). The gene encoding the florigen, the 14-3-3 protein, or the bZIP transcription factor may be a gene encoding a protein (mutant protein) having a mutation in at least one binding site among sites corresponding to D62 and the like in SEQ ID NO: 1, sites corresponding to F200 and the like in SEQ ID NO: 2, and sites corresponding to R189 to F195 in SEQ ID NO 3. A gene encoding a mutant protein may be produced by introducing a mutation using a genetic engineering technique well-known to a person skilled in the art such as a site-directed mutagenesis method. For example, a gene encoding a mutant protein may be produced by combining a PCR reaction, a restriction enzyme reaction, a ligation reaction, and the like. Specifically, a kit such as a QuikChange™ Site-Directed Mutagenesis Kit (STRATAGENE) may be used. The (over)expression of each of those genes in a plant body, preferably in a desired plant tissue cells, more preferably in cell organelles allows binding between a florigen and a 14-3-3 protein and/or binding between a 14-3-3 protein and a bZIP transcription factor to be promoted, and allows flowering to be promoted (accelerated).

A method of (over) expressing a gene is not particularly limited. In general, however, there is adopted a known method such as introducing an isolated gene into plant cells in an expressible manner by a genetic engineering technique. The known method is exemplified by a method involving introducing a recombinant vector containing a desired gene into a host. Specific examples thereof may include a method utilizing infection with Agrobacterium bacteria, such as a protoplast co-culture method or a leaf disk method, a polyethylene glycol method, an electroporation method, a microinjection method, a particle gun method, a liposome method, and an introduction method using an appropriate vector system, and an optimum method has only to be selected from the methods depending on the kind of host cells. It should be noted that examples of the vector include, but not particularly limited to, a plasmid, a phage, and a cosmid, and the vector has only to be appropriately selected depending on the kind of host cells. Further, a wide range of kinds of plants may be used as the host cells. In addition, the host cells may be proliferative plant materials such as protoplasts, cells, calli, organ leaves, seeds, germs, pollens, egg cells, and zygotes, or may be parts of a plant body such as flowers, fruits, leaves, roots, or rooted cuttings.

The promotion of flowering means shortening a vegetative growth period and accelerating the formation of flowers. It is considered that this allows picking seasons of agricultural and horticultural crops to be shifted (generally accelerated), and allows an improvement in efficiency of breeding and increases in yield of agricultural and horticultural crops to be achieved. Further, abilities to form flowers and seeds are estimated to be normal, and hence plant seeds can be quickly produced.

Further, examples of the substance capable of suppressing binding between a florigen and a 14-3-3 protein and/or binding between a 14-3-3 protein and a bZIP transcription factor include florigen, 14-3-3 protein, and bZIP transcription factor polypeptide fragments each having a binding ability, and modified proteins of a florigen and the like, which inhibit functions of normal proteins. The introduction of a gene expressing each of those substances into a plant body allows the formation of a florigen activation complex to be inhibited and allows flowering to be regulated. The “modification” as used herein means completely deleting any one of amino acid sequence regions characteristic of those proteins, and introducing deletions, substitutions, and/or insertions into one or several amino acid residues for a particular site of those sequence regions. The modified protein includes a mutant protein.

A method of modifying a gene encoding each protein has only to be performed by a conventionally known technique and is not particularly limited. That is, as mentioned above, the method has only to be performed, for example, by introducing a mutation into a base sequence utilizing a PCR method, by a known site-directed mutagenesis method (Kunkel et al.: Proc. Natl. Acad. Sci. USA, vol. 82. p 488- (1985)), or with a commercially available kit (e.g., Quikchange Site-Directed Mutagenesis Kit: STRATAGENE).

The modified protein (including a polypeptide) acts in a dominant manner on a protein obtained from a wild-type gene, and suppresses the transcription of a target gene to inhibit the expression of the target gene. Therefore, through the use of a modified gene, a function inherent in a wild-type gene is inhibited and lost, and a transformant in which flowering is suppressed can be produced. Hence, the time and effort required for the production of a transformant in which flowering is suppressed can be reduced.

A method of introducing a modified protein may be performed by the above-mentioned gene expression method.

Further, the introduction of a mutation into a binding site of an endogenous protein of a plant body, and the knockout of a protein may be performed by any of known methods utilizing an antisense method, a gene targeting method, a gene knockout tagging method, and the like (Kempin et al. Nature 389: 802-803. 1997).

The antisense method is a method involving constructing a vector containing an appropriate promoter and a target gene in a reverse direction (antisense DNA) disposed downstream of the promoter, and introducing the vector into a plant, thereby suppressing the expression of the target gene. The gene targeting method is a method involving inserting another DNA by homologous recombination into part of a target gene to knock out the target gene. The method of the present invention using the gene knockout tagging method is a method involving inserting T-DNA or a transposon into a genome at random and screening a target gene-knockout strain by utilizing PCR.

The suppression of flowering means extending a vegetative growth period and delaying the formation of flowers. It is considered that the suppression results in an increase in weight of a plant body as compared to a wild-type plant body (Development 135, 767-774 (2008) doi:10.1242/dev.008631, FIG. 3E). That is, the suppression of flowering allows a plant body having an increased amount of biomass to be produced per individual to be provided. The increased amount of biomass to be produced means that a total weight per individual is large as compared to a wild-type, and also includes the case where the weight of part of tissues of a plant body is specifically large and the weights of the other tissues are similar to those of a wild-type. Plant biomass is generated by fixation of carbon dioxide in air using solar energy, and hence can be trapped as so-called carbon neutral energy. An increase in plant biomass has effects of global environment conservation, global warming prevention, and greenhouse gas emission reduction. Further, for a plant in which parts other than organs involved in flower-bud formation (flowers or seeds) are used as foods and the like, increases in yield of agricultural and horticultural crops per individual can be achieved by increasing the weight of a plant body through the suppression of flowering.

(Transformant)

The present invention also encompasses a transformant having a desired gene introduced therein or a transformant having a desired gene knocked out. The transformant of the present invention includes one obtained in the method of regulating flowering of a plant. The “transformant” includes a cell, a tissue, an organ, and a protoplast as well as a plant individual. Further, organisms as targets of transformation are also not particularly limited and examples thereof may include various microorganisms (including Escherichia coli and the like) and plants. Further, animals and insects may also be used as targets of transformation by selecting promoters and vectors. Further, the transformant as used herein also includes a plant having introduced therein the gene according to the present invention or an offspring of the plant having the same properties as the plant, or a tissue thereof. Such transformant may be produced using the technique described in the above-mentioned “Method of regulating flowering of plant” section.

### EXAMPLES

Hereinafter, the present invention is specifically described by way of examples and experimental examples for further understanding of the present invention. However, it should be appreciated that the scope of the present invention is by no means limited by these examples and experimental examples. Further, all of Hd3a, GF14, and FD1 used in the following examples are proteins derived from rice. In the notation of the proteins, each of the proteins may be specified with or without the prefix “Os.” However, all of the proteins used in the following examples are proteins derived from rice.

First, a production method for a plasmid used in examples is shown below.

Full-length cDNAs for Hd3a (Os06g0157700), OsGF14 (OsGF14a: Os08g0480800, OsGF14b: Os04g0462500, OsGF14c: Os08g0430500, OsGF14d: Os11g0546900, OsGF14e: Os02g0580300, OsGF14f: Os03g0710800, OsGF14g: Os01g0209200, and OsGF14h: Os11g0609600), and OsFD1 (Os09g0540800) were cloned by RT-PCR. The coding regions were PCR-amplified by a conventional method and introduced into a pENTR/D-TOPO cloning vector (Invitrogen) to obtain entry clones.

The introduction of amino-acid substitutions or deletions into OsGF14, OsFD1, and Hd3a was performed by PCR with KOD FX DNA polymerase (TOYOBO). The PCR-amplified fragments were introduced into the pENTR/D-TOPO cloning vector to obtain entry clones.

pGII pUbq GW-T7 and pGII pUbq HA-GW were produced by inserting a ubiquitin promoter derived from maize, an NOS termination region (Miki and Shimamoto, Plant Cell Physiol. 45, 490 (2004).), and an attR recombination region (Nakagawa et al., 2007) with a T7 tag region or an HA tag region into a pGreen II vector (Hellens et al., Plant Mol. Biol. 42, 819 (2000).).

A pUbq:Hd3a-mCherry expression vector (Non Patent Literature 2) and pHd3a:Hd3a-GFP and prolC:Hd3a-GFP binary vectors (Tamaki et al., Science 316, 1033 (2007)) were produced based on the descriptions of the literatures. A prolC:Hd3a (R64G)-GFP binary vector and a prolC:Hd3a (R64G/R132A)-GFP binary vector were produced using mutant Hd3a cDNA as described in Tamaki et al., Science 316, 1033 (2007). In order to produce other vectors, a mutant Hd3a gene, a mutant OsGF14 gene, and a mutant OsFD1 gene were transformed into various vectors in pENTR D-TOPO vectors by a Gateway recombination method with Gateway BP clonase II (Invitrogen).

Those vectors may be included in pBTM116-GW and pVP16-GW in a yeast two-hybrid assay (Examples 8 and 9). Further, the vectors may be included in pGII pUbq GW-T7, pGII pUbq HA-GW, p35S-GFP-GW, p35S-mCerulean-GW, p35S-Vn-GW, p35S-Vc-GW, p35S-GW-Vn, p35S-GW-Vc, and pUbq-GW in a transient expression assay (Examples 11 and 12) and in a p2K-GW binary vector and a pANDA RNAi vector (Miki and Shimamoto 2004) in the production of a transgenic plant (Examples 10, 13-1, and 13-2. SV40 NLS was fused with GF14b and GF14e by PCR.

### Example 1

### Confirmation of Interactions Among Three Proteins

(1) Interactions among three proteins were confirmed by an in vitro GST pull-down assay.

A plasmid having incorporated therein cDNA encoding full-length Hd3a (residues 1 to 179) set forth in SEQ ID NO: 1 and cDNA encoding full-length GF14c (residues 1 to 256) set forth in SEQ ID NO: 2 was subjected to restriction enzyme treatment, and fragments obtained by the restriction enzyme treatment were fused with a polynucleotide encoding GST and introduced into a vector. The vector was introduced into Escherichia coli, and a GST-fused GF14c protein or a GST-fused Hd3a protein was expressed in Escherichia coli. After the culture of Escherichia coli, Escherichia coli was harvested by centrifugation, and the GST-fused GF14c protein or the GST-fused Hd3a protein was isolated and purified from the lysis solution.

10 nmol of each of the isolated and purified GST-fused GF14c protein and GST-fused Hd3a protein were incubated with 10 μl of a Glutathione Sepharose 4B resin (GE Healthcare) to bind each of the polypeptides to the resin. After that, the resin was washed with a phosphate buffer (pH 6.8) containing 50 mM KCl and 1 mM DTT to remove a free GST-fused protein unbound to the resin. The isolated and purified GST protein bound to the resin was used as a negative control. To three kinds of resins to which the GST-GF14c protein, the GST-Hd3a protein, and the GST protein were bound, 1 nmol of each of the isolated and purified proteins (OsFD1 having introduced therein a mutation of S192E in a region of positions 147 to 195 in SEQ ID NO: 3 and full-length Hd3a, both of which were not fused with GST) was added and the mixtures were incubated at room temperature for 15 minutes. After that, the resins were washed with a phosphate buffer (pH 6.8) containing 50 mM KCl and 1 mM DTT to remove a free GST-GF14c polypeptide unbound to the resins. Each of the bound proteins was eluted with a phosphate buffer (pH 6.8) containing 50 mM glutathione, 50 mM KCl, and 1 mM DTT and confirmed by SDS polyacrylamide electrophoresis.

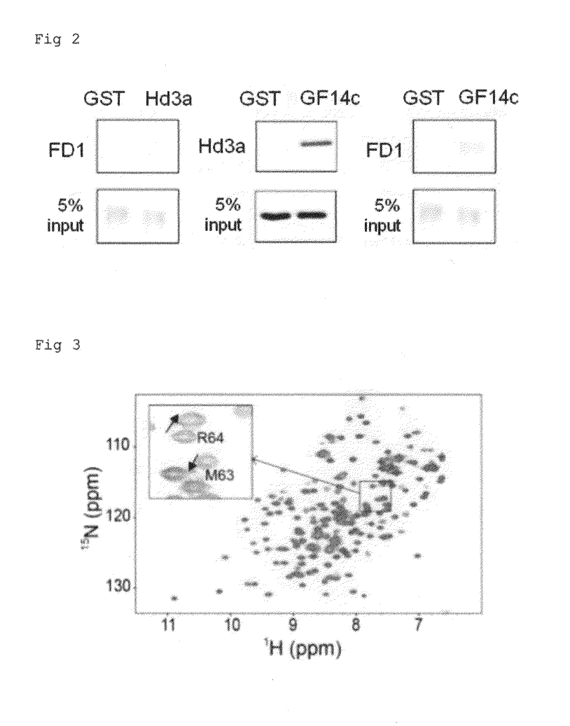

FIG. 2 shows the results. No interaction was observed between Hd3a and OsFD1 (FD1), whereas interactions were observed between Hd3a and GF14c and between GF14c and OsFD1, suggesting that Hd3a and OsFD1 were bound via GF14c.

(2) The interaction between Hd3a and GF14c was confirmed using NMR. A 15N stable isotope-labeled Hd3a protein solution was prepared with a 50 mM phosphate buffer (pH 6.8) containing 100 mM KCl, 1 mM DTT, and 7% deuterium oxide so as to achieve a final concentration of 0.2 mM. After that, 15N-HSQC NMR spectra of the following solutions were measured: the prepared Hd3a protein solution alone; and a mixed solution obtained by adding to the Hd3a protein solution a GF14c protein unlabeled with a stable isotope at a molar ratio of 1:0.5. The spectra were compared to each other. The NMR measurement was performed at a temperature of 30° C. and performed using an AV500 NMR apparatus manufactured by Bruker.

FIG. 3 shows the results. The NMR spectrum of Hd3a changed through binding with GF14c. Thus, binding between Hd3a and GF14c was confirmed. Further, the binding was found to be site-specific because the shift of NMR signals was partially (e.g., Hd3a R64 and M63 in the expanded view of FIG. 3) observed.

### Example 2

### Production of Crystals of Florigen Activation Complex

A plasmid having incorporated therein cDNA encoding residues 6 to 170 in full-length Hd3a (residues 1 to 179) set forth in SEQ ID NO: 1 and cDNA encoding residues 1 to 235 in full-length GF14c (residues 1 to 256) set forth in SEQ ID NO: 2 was subjected to restriction enzyme treatment, and fragments obtained by the restriction enzyme treatment were fused with a polynucleotide encoding GST and introduced into a vector. The vector was introduced into Escherichia coli. A GST-fused Hd3a protein and GF14c protein were expressed in Escherichia coli. After the culture of Escherichia coli, Escherichia coli was harvested by centrifugation, and the proteins were purified from the lysis solution. After that, analysis was performed by an SDS-polyacrylamide electrophoresis method (SDS-PAGE). As a result, the proteins were each found to have a purity of 95% or more. A polypeptide encoding residues 187 to 195 in full-length OsFD1 (residues 1 to 195) set forth in SEQ ID NO: 3 was prepared by chemical synthesis.

The resultant Hd3a and GF14c were mixed with each other at a molar ratio of 1:1.5 and dialyzed against a 10 mM Tris-HCl buffer (pH 7.5) containing 20 mM NaCl to prepare a sample of a complex. The crystallization of the complex was performed at 4° C. by a sitting drop method using a mixture of 1 μl of the protein solution (protein concentration: 10 mg/mL) and 1 μl of a precipitant solution (0.1 M HEPES (pH 7.5), 0.2 M ammonium sulfate, and 25% PEG 3350). After about 2 weeks, single crystals each measuring 0.1 by 0.1 mm were obtained. The obtained Hd3a-GF14c complex crystals were collected and incubated with the precipitant solution containing 25% ethylene glycol and a 2 mM OsFD1 polypeptide for 15 minutes to produce crystals of a florigen activation complex of Hd3a, GF14c, and OsFD1.

The resultant crystals were the following five kinds:

(florigen activation complex 1) a crystal having a space group of P1 and lattice constants of a=76.7 Å, b=96.6 Å, c=99.5 Å, α=68.2°, β87.9°, and γ=77.9° at a resolution of 2.4 Å;
(florigen activation complex 2) a crystal having a space group of P1 and lattice constants of a=76.8 Å, b=97.3 Å, c=99.8 Å, α=68.1°, β87.8°, and γ=77.9° at a resolution of 2.2 Å;
(florigen activation complex 3) a crystal having a space group of P1 and lattice constants of a=76.2 Å, b=96.1 Å, c=99.1 Å, α=68.2°, β=88.6°, and γ=77.8° at a resolution of 2.8 Å;
(florigen activation complex 4) a crystal having a space group of P6522 and lattice constants of a=129.0 Å, b=129.0 Å, c=342.0 Å, α=90°, β=90°, and γ=120° at a resolution of 2.85 Å; and
(florigen activation complex 5) a crystal having a space group of P4 and lattice constants of a=155.9 Å, b=155.9 Å, c=496.4 Å, α=90°, β=90°, and γ=90° at a resolution of 2.96 Å.

### Example 3

### X-Ray Structure Analysis of Crystals of Florigen Activation Complex

The resultant crystals were measured for their X-ray diffraction images using a CCD detector in the BL5A beamline of a synchrotron radiation research facility Photon Factory. Atomic coordinates of a florigen activation complex were obtained based on the resultant diffraction images.

X-ray diffraction data was collected to perform the indexing of individual diffraction spots and the calculation of diffraction intensities. Phase angles were determined from the resultant diffraction intensities and search models by a molecular replacement method. Electron density maps were derived by inverse Fourier transform based on the diffraction intensities of the diffraction spots and the phase angles described above. Atomic coordinates were constructed based on the resultant electron density maps.

Specifically, the resultant data was processed using HKL2000 and scaled with SCALEPACK. As a search model, the crystal structure of a florigen was determined by a molecular replacement method. The resultant model was refined at 2.4 Å using CNS and REFMAC. After each refinement, the resultant model was corrected with an electron density map 2Fo-Fc map using COOT.

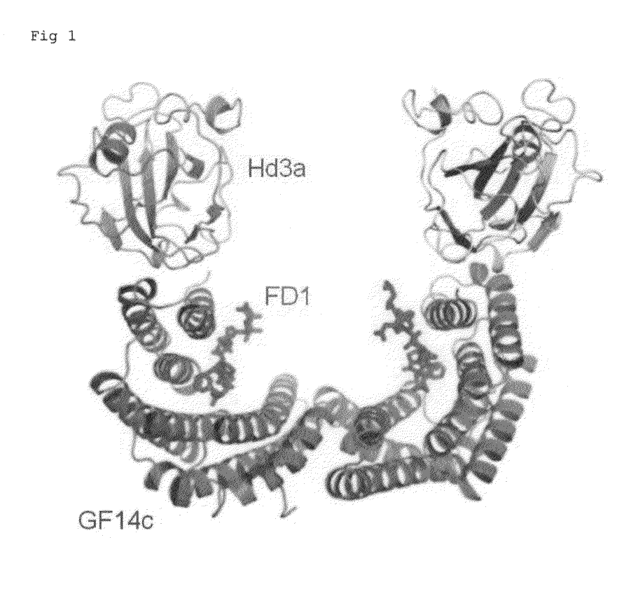

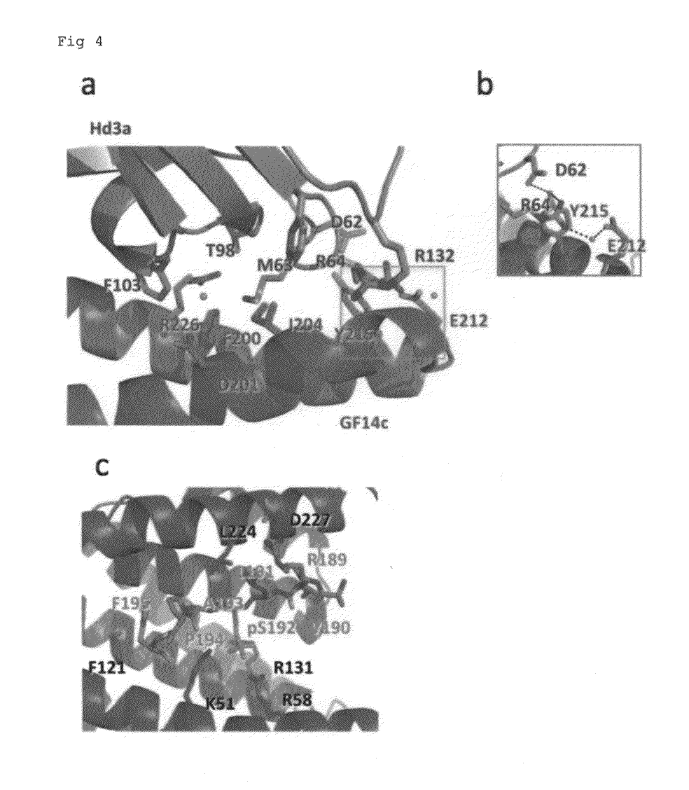

Table 1 shows atomic coordinates obtained from the crystal of the florigen activation complex 1. Further, FIGS. 1 and 4 illustrate conformation models constructed from the atomic coordinates. It is understood that phosphorylated 5192 (pS192) in OsFD1 is specifically recognized by GF14c.

Lengthy table referenced here
US20130019345A1-20130117-T00001
Please refer to the end of the specification for access instructions.

### Example 4

### Production of Crystals of Florigen

### (1) Production of Expression Vector

A plasmid having incorporated therein cDNA encoding residues 6 to 170 in full-length Hd3a (residues 1 to 179) set forth in SEQ ID NO: 1 was subjected to restriction enzyme treatment. Fragments obtained by the restriction enzyme treatment were fused with a polynucleotide encoding GST and introduced into a vector. The vector was introduced into Escherichia coli. A GST-fused Hd3a protein was expressed in Escherichia coli. After the culture of Escherichia coli, Escherichia coli was harvested by centrifugation, and the Hd3a protein was purified from the lysis solution. After that, analysis was performed by a SDS-polyacrylamide electrophoresis method (SDS-PAGE). As a result, the Hd3a protein was found to have a purity of 95% or more.

The purified Hd3a protein was dialyzed against a 10 mM Tris buffer (pH 7.5) containing 20 mM NaCl and concentrated so as to achieve a concentration of 5 mg/ml. Crystallization was performed at 4° C. by a sitting drop method using a mixture of 1 μl of the protein solution and 1 μl of a precipitant solution (0.1 M cacodylic acid (pH 6.5), 0.2 M ammonium tartrate, and 30% PEG 8000). Single crystals each measuring 0.1 by 0.1 mm were obtained after about 1 day.

The resultant crystals were the following two kinds:

(florigen 1) a crystal having a space group of P63 and lattice constants of a=65.9 Å, b=65.9 Å, c=59.8 Å, α=90°, β=90°, and γ=120° at a resolution of 1.3 Å; and
(florigen 2) a crystal having a space group of P6522 and lattice constants of a=66.0 Å, b=66.0 Å, c=60.2 Å, α=90°, β=90°, and γ=120° at a resolution of 1.4 Å.

### Example 5

### X-Ray Structure Analysis of Crystals of Florigen

The resultant crystals were measured for their X-ray diffraction images using a CCD detector in the BL5A beamline of a synchrotron radiation research facility Photon Factory. Atomic coordinates of a florigen were obtained based on the resultant diffraction images.

X-ray diffraction data was collected to perform the indexing of individual diffraction spots and the calculation of diffraction intensities. Phase angles were determined from the resultant diffraction intensities and search models by a molecular replacement method. Electron density maps were derived by inverse Fourier transform based on the diffraction intensities of the diffraction spots and the phase angles described above. Atomic coordinates were constructed based on the resultant electron density maps.

Specifically, the resultant data was processed using HKL2000 and scaled with SCALEPACK. As a search model, the crystal structure of a florigen was determined by a molecular replacement method. The resultant model was refined at 1.3 Å and 1.4 Å using CNS and REFMAC. After each refinement, the resultant model was corrected with an electron density map 2Fo-Fc map using COOT.

Table 2 below shows atomic coordinates obtained from the crystal of the florigen 1.

TABLE 2
Atomic coordinates of florigen

| | | | | | | | | | | | |
|---|---|---|---|---|---|---|---|---|---|---|---|
| ATOM | 1 | N | GLY | A | 5 | 8.706 | 4.683 | 11.752 | 1.00 | 46.29 | N |
| ATOM | 2 | CA | GLY | A | 5 | 9.393 | 3.368 | 11.940 | 1.00 | 46.24 | C |
| ATOM | 3 | C | GLY | A | 5 | 10.574 | 3.541 | 12.876 | 1.00 | 46.01 | C |
| ATOM | 4 | O | GLY | A | 5 | 10.375 | 3.941 | 14.027 | 1.00 | 46.10 | O |
| ATOM | 5 | N | ARG | A | 6 | 11.804 | 3.261 | 12.435 | 1.00 | 45.65 | N |
| ATOM | 6 | CA | ARG | A | 6 | 12.210 | 2.730 | 11.112 | 1.00 | 45.26 | C |
| ATOM | 7 | CB | ARG | A | 6 | 11.465 | 3.383 | 9.940 | 1.00 | 45.27 | C |
| ATOM | 8 | CG | ARG | A | 6 | 11.788 | 2.843 | 8.535 | 1.00 | 45.56 | C |
| ATOM | 9 | CD | ARG | A | 6 | 11.793 | 1.306 | 8.431 | 1.00 | 44.76 | C |
| ATOM | 10 | NE | ARG | A | 6 | 10.455 | 0.711 | 8.470 | 1.00 | 44.76 | N |
| ATOM | 11 | CZ | ARG | A | 6 | 10.174 | −0.544 | 8.112 | 1.00 | 44.36 | C |
| ATOM | 12 | NH1 | ARG | A | 6 | 11.133 | −1.355 | 7.673 | 1.00 | 43.31 | N |
| ATOM | 13 | NH2 | ARG | A | 6 | 8.926 | −0.989 | 8.181 | 1.00 | 42.93 | N |
| ATOM | 14 | C | ARG | A | 6 | 13.715 | 2.991 | 11.021 | 1.00 | 44.86 | C |
| ATOM | 15 | O | ARG | A | 6 | 14.443 | 2.270 | 10.333 | 1.00 | 44.69 | O |
| ATOM | 16 | N | ALA | A | 7 | 14.138 | 4.063 | 11.699 | 1.00 | 44.40 | N |
| ATOM | 17 | CA | ALA | A | 7 | 15.500 | 4.266 | 12.224 | 1.00 | 43.74 | C |
| ATOM | 18 | CB | ALA | A | 7 | 15.950 | 3.064 | 13.070 | 1.00 | 43.95 | C |
| ATOM | 19 | C | ALA | A | 7 | 16.621 | 4.709 | 11.283 | 1.00 | 43.16 | C |
| ATOM | 20 | O | ALA | A | 7 | 17.202 | 3.903 | 10.552 | 1.00 | 43.44 | O |
| ATOM | 21 | N | ARG | A | 8 | 16.913 | 6.009 | 11.342 | 1.00 | 42.15 | N |
| ATOM | 22 | CA | ARG | A | 8 | 18.164 | 6.599 | 10.848 | 1.00 | 40.76 | C |
| ATOM | 23 | CB | ARG | A | 8 | 19.375 | 5.874 | 11.472 | 1.00 | 40.84 | C |
| ATOM | 24 | CG | ARG | A | 8 | 20.377 | 6.769 | 12.212 | 1.00 | 40.73 | C |
| ATOM | 25 | CD | ARG | A | 8 | 21.157 | 7.706 | 11.292 | 1.00 | 41.25 | C |
| ATOM | 26 | NE | ARG | A | 8 | 21.484 | 7.093 | 10.003 | 1.00 | 41.84 | N |
| ATOM | 27 | CZ | ARG | A | 8 | 22.667 | 6.576 | 9.682 | 1.00 | 43.45 | C |
| ATOM | 28 | NH1 | ARG | A | 8 | 22.849 | 6.045 | 8.478 | 1.00 | 43.18 | N |
| ATOM | 29 | NH2 | ARG | A | 8 | 23.671 | 6.582 | 10.554 | 1.00 | 43.26 | N |
| ATOM | 30 | C | ARG | A | 8 | 18.310 | 6.740 | 9.314 | 1.00 | 40.13 | C |
| ATOM | 31 | O | ARG | A | 8 | 19.319 | 6.320 | 8.742 | 1.00 | 40.05 | O |
| ATOM | 32 | N | ASP | A | 9 | 17.323 | 7.356 | 8.652 | 1.00 | 39.15 | N |
| ATOM | 33 | CA | ASP | A | 9 | 17.456 | 7.694 | 7.230 | 1.00 | 37.79 | C |
| ATOM | 34 | CB | ASP | A | 9 | 16.125 | 8.171 | 6.626 | 1.00 | 38.31 | C |
| ATOM | 35 | CG | ASP | A | 9 | 14.930 | 7.384 | 7.110 | 1.00 | 39.78 | C |
| ATOM | 36 | OD1 | ASP | A | 9 | 13.962 | 8.033 | 7.568 | 1.00 | 41.40 | O |
| ATOM | 37 | OD2 | ASP | A | 9 | 14.943 | 6.135 | 7.015 | 1.00 | 41.38 | O |
| ATOM | 38 | C | ASP | A | 9 | 18.475 | 8.823 | 7.061 | 1.00 | 36.45 | C |
| ATOM | 39 | O | ASP | A | 9 | 18.967 | 9.371 | 8.045 | 1.00 | 36.65 | O |
| ATOM | 40 | N | PRO | A | 10 | 18.799 | 9.184 | 5.809 | 1.00 | 34.65 | N |
| ATOM | 41 | CA | PRO | A | 10 | 19.433 | 10.483 | 5.641 | 1.00 | 33.25 | C |
| ATOM | 42 | CB | PRO | A | 10 | 19.729 | 10.528 | 4.142 | 1.00 | 32.98 | C |
| ATOM | 43 | CG | PRO | A | 10 | 19.779 | 9.089 | 3.730 | 1.00 | 34.18 | C |
| ATOM | 44 | CD | PRO | A | 10 | 18.692 | 8.465 | 4.528 | 1.00 | 34.61 | C |
| ATOM | 45 | C | PRO | A | 10 | 18.500 | 11.636 | 6.065 | 1.00 | 31.38 | C |
| ATOM | 46 | O | PRO | A | 10 | 18.947 | 12.775 | 6.155 | 1.00 | 31.77 | O |
| ATOM | 47 | N | LEU | A | 11 | 17.225 | 11.336 | 6.316 | 1.00 | 29.48 | N |
| ATOM | 48 | CA | LEU | A | 11 | 16.274 | 12.345 | 6.813 | 1.00 | 27.68 | C |
| ATOM | 49 | CB | LEU | A | 11 | 14.816 | 11.916 | 6.588 | 1.00 | 26.63 | C |
| ATOM | 50 | CG | LEU | A | 11 | 14.223 | 11.864 | 5.168 | 1.00 | 25.43 | C |
| ATOM | 51 | CD1 | LEU | A | 11 | 12.770 | 11.460 | 5.229 | 1.00 | 23.05 | C |
| ATOM | 52 | CD2 | LEU | A | 11 | 14.346 | 13.191 | 4.424 | 1.00 | 23.76 | C |
| ATOM | 53 | C | LEU | A | 11 | 16.500 | 12.672 | 8.288 | 1.00 | 27.42 | C |
| ATOM | 54 | O | LEU | A | 11 | 16.226 | 13.794 | 8.730 | 1.00 | 25.74 | O |
| ATOM | 55 | N | VAL | A | 12 | 16.976 | 11.695 | 9.059 | 1.00 | 27.11 | N |
| ATOM | 56 | CA | VAL | A | 12 | 17.276 | 11.955 | 10.468 | 1.00 | 27.10 | C |
| ATOM | 57 | CB | VAL | A | 12 | 17.000 | 10.742 | 11.410 | 1.00 | 27.47 | C |
| ATOM | 58 | CG1 | VAL | A | 12 | 16.029 | 9.764 | 10.781 | 1.00 | 27.69 | C |
| ATOM | 59 | CG2 | VAL | A | 12 | 18.287 | 10.049 | 11.820 | 1.00 | 28.05 | C |
| ATOM | 60 | C | VAL | A | 12 | 18.697 | 12.474 | 10.605 | 1.00 | 27.02 | C |
| ATOM | 61 | O | VAL | A | 12 | 18.943 | 13.378 | 11.399 | 1.00 | 26.52 | O |
| ATOM | 62 | N | VAL | A | 13 | 19.619 | 11.920 | 9.813 | 1.00 | 26.34 | N |
| ATOM | 63 | CA | VAL | A | 13 | 20.974 | 12.455 | 9.688 | 1.00 | 26.40 | C |
| ATOM | 64 | CB | VAL | A | 13 | 21.832 | 11.636 | 8.672 | 1.00 | 26.94 | C |
| ATOM | 65 | CG1 | VAL | A | 13 | 23.016 | 12.446 | 8.154 | 1.00 | 27.65 | C |
| ATOM | 66 | CG2 | VAL | A | 13 | 22.314 | 10.349 | 9.295 | 1.00 | 27.69 | C |
| ATOM | 67 | C | VAL | A | 13 | 20.891 | 13.917 | 9.239 | 1.00 | 25.34 | C |
| ATOM | 68 | O | VAL | A | 13 | 21.604 | 14.782 | 9.758 | 1.00 | 25.67 | O |
| ATOM | 69 | N | GLY | A | 14 | 20.002 | 14.186 | 8.279 | 1.00 | 24.50 | N |
| ATOM | 70 | CA | GLY | A | 14 | 19.808 | 15.539 | 7.763 | 1.00 | 22.95 | C |
| ATOM | 71 | C | GLY | A | 14 | 18.975 | 16.440 | 8.656 | 1.00 | 21.25 | C |
| ATOM | 72 | O | GLY | A | 14 | 18.804 | 17.619 | 8.343 | 1.00 | 21.51 | O |
| ATOM | 73 | N | ARG | A | 15 | 18.471 | 15.890 | 9.763 | 1.00 | 20.71 | N |
| ATOM | 74 | CA | ARG | A | 15 | 17.691 | 16.624 | 10.770 | 1.00 | 19.32 | C |
| ATOM | 75 | CB | ARG | A | 15 | 18.516 | 17.757 | 11.419 | 1.00 | 19.74 | C |
| ATOM | 76 | CG | ARG | A | 15 | 19.854 | 17.337 | 12.057 | 1.00 | 22.06 | C |
| ATOM | 77 | CD | ARG | A | 15 | 19.672 | 16.149 | 12.971 | 1.00 | 27.33 | C |
| ATOM | 78 | NE | ARG | A | 15 | 20.898 | 15.788 | 13.677 | 1.00 | 31.03 | N |
| ATOM | 79 | CZ | ARG | A | 15 | 21.086 | 14.638 | 14.326 | 1.00 | 32.99 | C |
| ATOM | 80 | NH1 | ARG | A | 15 | 20.134 | 13.708 | 14.357 | 1.00 | 33.51 | N |
| ATOM | 81 | NH2 | ARG | A | 15 | 22.236 | 14.413 | 14.940 | 1.00 | 34.74 | N |
| ATOM | 82 | C | ARG | A | 15 | 16.344 | 17.158 | 10.256 | 1.00 | 18.54 | C |
| ATOM | 83 | O | ARG | A | 15 | 15.694 | 17.977 | 10.915 | 1.00 | 16.85 | O |
| ATOM | 84 | N | VAL | A | 16 | 15.926 | 16.676 | 9.088 | 1.00 | 17.20 | N |
| ATOM | 85 | CA | VAL | A | 16 | 14.594 | 16.981 | 8.554 | 1.00 | 16.42 | C |
| ATOM | 86 | CB | VAL | A | 16 | 14.452 | 16.497 | 7.111 | 1.00 | 16.16 | C |
| ATOM | 87 | CG1 | VAL | A | 16 | 13.025 | 16.678 | 6.618 | 1.00 | 15.31 | C |
| ATOM | 88 | CG2 | VAL | A | 16 | 15.429 | 17.228 | 6.198 | 1.00 | 16.22 | C |
| ATOM | 89 | C | VAL | A | 16 | 13.529 | 16.339 | 9.438 | 1.00 | 16.42 | C |
| ATOM | 90 | O | VAL | A | 16 | 12.586 | 17.007 | 9.861 | 1.00 | 14.92 | O |
| ATOM | 91 | N | VAL | A | 17 | 13.699 | 15.042 | 9.741 | 1.00 | 17.05 | N |
| ATOM | 92 | CA | VAL | A | 17 | 12.971 | 14.429 | 10.836 | 1.00 | 18.33 | C |
| ATOM | 93 | CB | VAL | A | 17 | 12.979 | 12.880 | 10.776 | 1.00 | 18.85 | C |
| ATOM | 94 | CG1 | VAL | A | 17 | 12.460 | 12.297 | 12.090 | 1.00 | 20.27 | C |
| ATOM | 95 | CG2 | VAL | A | 17 | 12.130 | 12.398 | 9.592 | 1.00 | 19.66 | C |
| ATOM | 96 | C | VAL | A | 17 | 13.664 | 14.958 | 12.096 | 1.00 | 18.52 | C |
| ATOM | 97 | O | VAL | A | 17 | 14.863 | 14.725 | 12.292 | 1.00 | 19.61 | O |
| ATOM | 98 | N | GLY | A | 18 | 12.908 | 15.699 | 12.902 | 1.00 | 18.19 | N |
| ATOM | 99 | CA | GLY | A | 18 | 13.460 | 16.496 | 13.984 | 1.00 | 18.34 | C |
| ATOM | 100 | C | GLY | A | 18 | 13.054 | 17.950 | 13.814 | 1.00 | 17.75 | C |
| ATOM | 101 | O | GLY | A | 18 | 12.180 | 18.441 | 14.531 | 1.00 | 19.12 | O |
| ATOM | 102 | N | ASP | A | 19 | 13.671 | 18.636 | 12.854 | 1.00 | 16.90 | N |
| ATOM | 103 | CA | ASP | A | 19 | 13.386 | 20.057 | 12.654 | 1.00 | 15.56 | C |
| ATOM | 104 | CB | ASP | A | 19 | 14.466 | 20.725 | 11.810 | 1.00 | 15.27 | C |
| ATOM | 105 | CG | ASP | A | 19 | 15.803 | 20.825 | 12.521 | 1.00 | 15.22 | C |
| ATOM | 106 | OD1 | ASP | A | 19 | 15.879 | 20.650 | 13.763 | 1.00 | 18.15 | O |
| ATOM | 107 | OD2 | ASP | A | 19 | 16.786 | 21.090 | 11.814 | 1.00 | 14.86 | O |
| ATOM | 108 | C | ASP | A | 19 | 12.040 | 20.309 | 11.991 | 1.00 | 14.92 | C |
| ATOM | 109 | O | ASP | A | 19 | 11.388 | 21.298 | 12.292 | 1.00 | 16.18 | O |
| ATOM | 110 | N | VAL | A | 20 | 11.630 | 19.418 | 11.093 | 1.00 | 13.95 | N |
| ATOM | 111 | CA | VAL | A | 20 | 10.422 | 19.676 | 10.306 | 1.00 | 13.45 | C |
| ATOM | 112 | CB | VAL | A | 20 | 10.761 | 19.793 | 8.804 | 1.00 | 12.68 | C |
| ATOM | 113 | CG1 | VAL | A | 20 | 9.490 | 20.028 | 7.991 | 1.00 | 11.79 | C |
| ATOM | 114 | CG2 | VAL | A | 20 | 11.789 | 20.907 | 8.550 | 1.00 | 14.23 | C |
| ATOM | 115 | C | VAL | A | 20 | 9.382 | 18.583 | 10.489 | 1.00 | 13.69 | C |
| ATOM | 116 | O | VAL | A | 20 | 8.184 | 18.864 | 10.607 | 1.00 | 13.87 | O |
| ATOM | 117 | N | LEU | A | 21 | 9.865 | 17.342 | 10.540 | 1.00 | 14.73 | N |
| ATOM | 118 | CA | LEU | A | 21 | 8.987 | 16.190 | 10.556 | 1.00 | 15.26 | C |
| ATOM | 119 | CB | LEU | A | 21 | 9.166 | 15.364 | 9.277 | 1.00 | 14.80 | C |
| ATOM | 120 | CG | LEU | A | 21 | 8.941 | 16.094 | 7.945 | 1.00 | 12.27 | C |
| ATOM | 121 | CD1 | LEU | A | 21 | 9.251 | 15.176 | 6.794 | 1.00 | 12.76 | C |
| ATOM | 122 | CD2 | LEU | A | 21 | 7.554 | 16.679 | 7.823 | 1.00 | 12.86 | C |
| ATOM | 123 | C | LEU | A | 21 | 9.253 | 15.308 | 11.752 | 1.00 | 16.68 | C |
| ATOM | 124 | O | LEU | A | 21 | 10.353 | 15.303 | 12.306 | 1.00 | 16.92 | O |
| ATOM | 125 | N | ASP | A | 22 | 8.219 | 14.563 | 12.111 | 1.00 | 17.80 | N |
| ATOM | 126 | CA | ASP | A | 22 | 8.346 | 13.448 | 13.039 | 1.00 | 19.01 | C |
| ATOM | 127 | CB | ASP | A | 22 | 7.083 | 13.333 | 13.882 | 1.00 | 19.32 | C |
| ATOM | 128 | CG | ASP | A | 22 | 6.985 | 14.440 | 14.926 | 1.00 | 20.48 | C |
| ATOM | 129 | OD1 | ASP | A | 22 | 8.033 | 14.986 | 15.317 | 1.00 | 24.05 | O |
| ATOM | 130 | OD2 | ASP | A | 22 | 5.868 | 14.782 | 15.330 | 1.00 | 23.30 | O |
| ATOM | 131 | C | ASP | A | 22 | 8.608 | 12.186 | 12.239 | 1.00 | 20.19 | C |
| ATOM | 132 | O | ASP | A | 22 | 8.469 | 12.173 | 11.001 | 1.00 | 20.87 | O |
| ATOM | 133 | N | ALA | A | 23 | 9.008 | 11.130 | 12.941 | 1.00 | 20.00 | N |
| ATOM | 134 | CA | ALA | A | 23 | 9.253 | 9.829 | 12.338 | 1.00 | 20.49 | C |
| ATOM | 135 | CB | ALA | A | 23 | 9.538 | 8.795 | 13.419 | 1.00 | 20.19 | C |
| ATOM | 136 | C | ALA | A | 23 | 8.059 | 9.395 | 11.525 | 1.00 | 19.62 | C |
| ATOM | 137 | O | ALA | A | 23 | 6.915 | 9.584 | 11.940 | 1.00 | 20.07 | O |
| ATOM | 138 | N | PHE | A | 24 | 8.347 | 8.827 | 10.354 | 1.00 | 20.67 | N |
| ATOM | 139 | CA | PHE | A | 24 | 7.310 | 8.278 | 9.475 | 1.00 | 21.24 | C |
| ATOM | 140 | CB | PHE | A | 24 | 6.691 | 9.382 | 8.573 | 1.00 | 21.46 | C |
| ATOM | 141 | CG | PHE | A | 24 | 7.599 | 9.878 | 7.481 | 1.00 | 20.91 | C |
| ATOM | 142 | CD1 | PHE | A | 24 | 8.475 | 10.933 | 7.714 | 1.00 | 21.48 | C |
| ATOM | 143 | CE1 | PHE | A | 24 | 9.318 | 11.396 | 6.704 | 1.00 | 20.22 | C |
| ATOM | 144 | CZ | PHE | A | 24 | 9.273 | 10.814 | 5.443 | 1.00 | 20.95 | C |
| ATOM | 145 | CE2 | PHE | A | 24 | 8.398 | 9.772 | 5.190 | 1.00 | 20.11 | C |
| ATOM | 146 | CD2 | PHE | A | 24 | 7.564 | 9.308 | 6.204 | 1.00 | 20.15 | C |
| ATOM | 147 | C | PHE | A | 24 | 7.834 | 7.104 | 8.636 | 1.00 | 21.22 | C |
| ATOM | 148 | O | PHE | A | 24 | 9.045 | 6.933 | 8.449 | 1.00 | 22.17 | O |
| ATOM | 149 | N | VAL | A | 25 | 6.917 | 6.306 | 8.108 | 1.00 | 22.54 | N |
| ATOM | 150 | CA | VAL | A | 25 | 7.291 | 5.228 | 7.186 | 1.00 | 23.16 | C |
| ATOM | 151 | CB | VAL | A | 25 | 6.736 | 3.831 | 7.637 | 1.00 | 23.86 | C |
| ATOM | 152 | CG1 | VAL | A | 25 | 5.285 | 3.923 | 8.093 | 1.00 | 25.21 | C |
| ATOM | 153 | CG2 | VAL | A | 25 | 6.915 | 2.782 | 6.548 | 1.00 | 24.61 | C |
| ATOM | 154 | C | VAL | A | 25 | 6.897 | 5.604 | 5.749 | 1.00 | 22.57 | C |
| ATOM | 155 | O | VAL | A | 25 | 5.761 | 5.981 | 5.494 | 1.00 | 23.29 | O |
| ATOM | 156 | N | ARG | A | 26 | 7.864 | 5.544 | 4.841 | 1.00 | 22.73 | N |
| ATOM | 157 | CA | ARG | A | 26 | 7.631 | 5.866 | 3.435 | 1.00 | 21.98 | C |
| ATOM | 158 | CB | ARG | A | 26 | 8.945 | 5.827 | 2.654 | 1.00 | 22.63 | C |
| ATOM | 159 | CG | ARG | A | 26 | 9.985 | 6.859 | 3.069 | 1.00 | 22.46 | C |
| ATOM | 160 | CD | ARG | A | 26 | 11.343 | 6.518 | 2.484 | 1.00 | 24.06 | C |
| ATOM | 161 | NE | ARG | A | 26 | 12.366 | 7.534 | 2.740 | 1.00 | 26.16 | N |
| ATOM | 162 | CZ | ARG | A | 26 | 13.118 | 7.594 | 3.838 | 1.00 | 27.74 | C |
| ATOM | 163 | NH1 | ARG | A | 26 | 12.959 | 6.708 | 4.817 | 1.00 | 28.99 | N |
| ATOM | 164 | NH2 | ARG | A | 26 | 14.038 | 8.541 | 3.953 | 1.00 | 28.55 | N |
| ATOM | 165 | C | ARG | A | 26 | 6.635 | 4.870 | 2.852 | 1.00 | 22.09 | C |
| ATOM | 166 | O | ARG | A | 26 | 6.716 | 3.663 | 3.148 | 1.00 | 22.31 | O |
| ATOM | 167 | N | SER | A | 27 | 5.691 | 5.359 | 2.046 | 1.00 | 21.66 | N |
| ATOM | 168 | CA | SER | A | 27 | 4.698 | 4.475 | 1.413 | 1.00 | 20.78 | C |
| ATOM | 169 | CB | SER | A | 27 | 3.442 | 4.381 | 2.269 | 1.00 | 21.01 | C |
| ATOM | 170 | OG | SER | A | 27 | 2.776 | 5.632 | 2.331 | 1.00 | 22.05 | O |
| ATOM | 171 | C | SER | A | 27 | 4.315 | 4.863 | −0.021 | 1.00 | 21.09 | C |
| ATOM | 172 | O | SER | A | 27 | 3.502 | 4.189 | −0.659 | 1.00 | 21.20 | O |
| ATOM | 173 | N | THR | A | 28 | 4.878 | 5.957 | −0.513 | 1.00 | 20.27 | N |
| ATOM | 174 | CA | THR | A | 28 | 4.651 | 6.366 | −1.884 | 1.00 | 19.84 | C |
| ATOM | 175 | CB | THR | A | 28 | 3.536 | 7.430 | −1.989 | 1.00 | 19.89 | C |
| ATOM | 176 | OG1 | THR | A | 28 | 2.291 | 6.839 | −1.602 | 1.00 | 22.54 | O |
| ATOM | 177 | CG2 | THR | A | 28 | 3.396 | 7.989 | −3.416 | 1.00 | 19.83 | C |
| ATOM | 178 | C | THR | A | 28 | 5.963 | 6.912 | −2.356 | 1.00 | 19.45 | C |
| ATOM | 179 | O | THR | A | 28 | 6.668 | 7.586 | −1.602 | 1.00 | 20.19 | O |
| ATOM | 180 | N | ASN | A | 29 | 6.308 | 6.581 | −3.590 | 1.00 | 18.69 | N |
| ATOM | 181 | CA | ASN | A | 29 | 7.517 | 7.079 | −4.189 | 1.00 | 18.29 | C |
| ATOM | 182 | CB | ASN | A | 29 | 8.015 | 6.114 | −5.277 | 1.00 | 19.37 | C |
| ATOM | 183 | CG | ASN | A | 29 | 8.370 | 4.721 | −4.709 | 1.00 | 20.85 | C |
| ATOM | 184 | OD1 | ASN | A | 29 | 7.979 | 3.682 | −5.256 | 1.00 | 21.94 | O |
| ATOM | 185 | ND2 | ASN | A | 29 | 9.092 | 4.707 | −3.592 | 1.00 | 22.85 | N |
| ATOM | 186 | C | ASN | A | 29 | 7.349 | 8.541 | −4.637 | 1.00 | 17.79 | C |
| ATOM | 187 | O | ASN | A | 29 | 6.278 | 8.978 | −5.088 | 1.00 | 16.90 | O |
| ATOM | 188 | N | LEU | A | 30 | 8.417 | 9.294 | −4.422 | 1.00 | 16.34 | N |
| ATOM | 189 | CA | LEU | A | 30 | 8.494 | 10.699 | −4.743 | 1.00 | 15.26 | C |
| ATOM | 190 | CB | LEU | A | 30 | 8.590 | 11.503 | −3.448 | 1.00 | 15.20 | C |
| ATOM | 191 | CG | LEU | A | 30 | 8.821 | 13.013 | −3.506 | 1.00 | 14.70 | C |
| ATOM | 192 | CD1 | LEU | A | 30 | 7.612 | 13.720 | −4.106 | 1.00 | 13.91 | C |
| ATOM | 193 | CD2 | LEU | A | 30 | 9.103 | 13.540 | −2.110 | 1.00 | 15.07 | C |
| ATOM | 194 | C | LEU | A | 30 | 9.768 | 10.865 | −5.520 | 1.00 | 14.75 | C |
| ATOM | 195 | O | LEU | A | 30 | 10.820 | 10.418 | −5.079 | 1.00 | 15.24 | O |
| ATOM | 196 | N | LYS | A | 31 | 9.681 | 11.515 | −6.673 | 1.00 | 14.21 | N |
| ATOM | 197 | CA | LYS | A | 31 | 10.836 | 11.703 | −7.515 | 1.00 | 13.67 | C |
| ATOM | 198 | CB | LYS | A | 31 | 10.774 | 10.760 | −8.723 | 1.00 | 14.85 | C |
| ATOM | 199 | CG | LYS | A | 31 | 11.932 | 10.899 | −9.662 | 1.00 | 15.89 | C |
| ATOM | 200 | CD | LYS | A | 31 | 11.819 | 9.944 | −10.838 | 1.00 | 19.52 | C |
| ATOM | 201 | CE | LYS | A | 31 | 12.921 | 10.178 | −11.844 | 1.00 | 22.81 | C |
| ATOM | 202 | NZ | LYS | A | 31 | 12.789 | 9.222 | −12.987 | 1.00 | 27.06 | N |
| ATOM | 203 | C | LYS | A | 31 | 10.854 | 13.156 | −7.935 | 1.00 | 13.16 | C |
| ATOM | 204 | O | LYS | A | 31 | 9.966 | 13.601 | −8.658 | 1.00 | 13.34 | O |
| ATOM | 205 | N | VAL | A | 32 | 11.860 | 13.882 | −7.458 | 1.00 | 12.62 | N |
| ATOM | 206 | CA | VAL | A | 32 | 11.998 | 15.309 | −7.722 | 1.00 | 11.96 | C |
| ATOM | 207 | CB | VAL | A | 32 | 12.264 | 16.078 | −6.395 | 1.00 | 11.66 | C |
| ATOM | 208 | CG1 | VAL | A | 32 | 12.475 | 17.570 | −6.663 | 1.00 | 11.11 | C |
| ATOM | 209 | CG2 | VAL | A | 32 | 11.137 | 15.844 | −5.403 | 1.00 | 11.90 | C |
| ATOM | 210 | C | VAL | A | 32 | 13.191 | 15.509 | −8.658 | 1.00 | 12.56 | C |
| ATOM | 211 | O | VAL | A | 32 | 14.326 | 15.142 | −8.314 | 1.00 | 13.46 | O |
| ATOM | 212 | N | THR | A | 33 | 12.956 | 16.076 | −9.839 | 1.00 | 11.70 | N |
| ATOM | 213 | CA | THR | A | 33 | 13.988 | 16.182 | −10.858 | 1.00 | 12.55 | C |
| ATOM | 214 | CB | THR | A | 33 | 13.748 | 15.161 | −12.019 | 1.00 | 12.70 | C |
| ATOM | 215 | OG1 | THR | A | 33 | 13.542 | 13.854 | −11.471 | 1.00 | 13.48 | O |
| ATOM | 216 | CG2 | THR | A | 33 | 14.918 | 15.097 | −12.978 | 1.00 | 14.67 | C |
| ATOM | 217 | C | THR | A | 33 | 14.116 | 17.599 | −11.399 | 1.00 | 11.41 | C |
| ATOM | 218 | O | THR | A | 33 | 13.142 | 18.205 | −11.854 | 1.00 | 11.41 | O |
| ATOM | 219 | N | TYR | A | 34 | 15.337 | 18.119 | −11.343 | 1.00 | 11.75 | N |
| ATOM | 220 | CA | TYR | A | 34 | 15.698 | 19.371 | −11.959 | 1.00 | 12.44 | C |
| ATOM | 221 | CB | TYR | A | 34 | 16.520 | 20.211 | −10.995 | 1.00 | 12.68 | C |
| ATOM | 222 | CG | TYR | A | 34 | 15.756 | 20.795 | −9.820 | 1.00 | 10.94 | C |
| ATOM | 223 | CD1 | TYR | A | 34 | 15.057 | 21.978 | −9.959 | 1.00 | 11.31 | C |
| ATOM | 224 | CE1 | TYR | A | 34 | 14.378 | 22.546 | −8.880 | 1.00 | 9.98 | C |
| ATOM | 225 | CZ | TYR | A | 34 | 14.432 | 21.943 | −7.660 | 1.00 | 11.59 | C |
| ATOM | 226 | OH | TYR | A | 34 | 13.787 | 22.548 | −6.589 | 1.00 | 10.34 | O |
| ATOM | 227 | CE2 | TYR | A | 34 | 15.138 | 20.768 | −7.479 | 1.00 | 11.29 | C |
| ATOM | 228 | CD2 | TYR | A | 34 | 15.806 | 20.193 | −8.570 | 1.00 | 11.35 | C |
| ATOM | 229 | C | TYR | A | 34 | 16.551 | 19.057 | −13.178 | 1.00 | 13.57 | C |
| ATOM | 230 | O | TYR | A | 34 | 17.664 | 18.553 | −13.029 | 1.00 | 13.66 | O |
| ATOM | 231 | N | GLY | A | 35 | 16.033 | 19.344 | −14.374 | 1.00 | 13.84 | N |
| ATOM | 232 | CA | GLY | A | 35 | 16.732 | 18.958 | −15.616 | 1.00 | 15.18 | C |
| ATOM | 233 | C | GLY | A | 35 | 16.800 | 17.440 | −15.705 | 1.00 | 16.38 | C |
| ATOM | 234 | O | GLY | A | 35 | 15.775 | 16.761 | −15.785 | 1.00 | 16.96 | O |
| ATOM | 235 | N | SER | A | 36 | 18.010 | 16.896 | −15.662 | 1.00 | 17.77 | N |
| ATOM | 236 | CA | SER | A | 36 | 18.150 | 15.438 | −15.603 | 1.00 | 18.97 | C |
| ATOM | 237 | CB | SER | A | 36 | 19.090 | 14.942 | −16.696 | 1.00 | 19.64 | C |
| ATOM | 238 | OG | SER | A | 36 | 20.385 | 15.464 | −16.506 | 1.00 | 21.57 | O |
| ATOM | 239 | C | SER | A | 36 | 18.637 | 14.949 | −14.238 | 1.00 | 19.73 | C |
| ATOM | 240 | O | SER | A | 36 | 18.903 | 13.764 | −14.047 | 1.00 | 20.43 | O |
| ATOM | 241 | N | LYS | A | 37 | 18.728 | 15.869 | −13.281 | 1.00 | 19.10 | N |
| ATOM | 242 | CA | LYS | A | 37 | 19.241 | 15.559 | −11.950 | 1.00 | 20.22 | C |
| ATOM | 243 | CB | LYS | A | 37 | 20.023 | 16.752 | −11.382 | 1.00 | 19.94 | C |
| ATOM | 244 | CG | LYS | A | 37 | 21.147 | 17.305 | −12.241 | 1.00 | 23.65 | C |
| ATOM | 245 | CD | LYS | A | 37 | 21.718 | 18.564 | −11.584 | 1.00 | 23.68 | C |
| ATOM | 246 | CE | LYS | A | 37 | 22.729 | 19.279 | −12.471 | 1.00 | 28.46 | C |
| ATOM | 247 | NZ | LYS | A | 37 | 23.991 | 18.497 | −12.635 | 1.00 | 29.30 | N |
| ATOM | 248 | C | LYS | A | 37 | 18.100 | 15.234 | −10.999 | 1.00 | 19.27 | C |
| ATOM | 249 | O | LYS | A | 37 | 17.367 | 16.135 | −10.580 | 1.00 | 17.57 | O |
| ATOM | 250 | N | THR | A | 38 | 17.958 | 13.963 | −10.640 | 1.00 | 18.79 | N |
| ATOM | 251 | CA | THR | A | 38 | 17.002 | 13.549 | −9.619 | 1.00 | 18.53 | C |
| ATOM | 252 | CB | THR | A | 38 | 16.510 | 12.088 | −9.837 | 1.00 | 19.21 | C |
| ATOM | 253 | OG1 | THR | A | 38 | 15.766 | 12.018 | −11.064 | 1.00 | 19.55 | O |
| ATOM | 254 | CG2 | THR | A | 38 | 15.620 | 11.605 | −8.678 | 1.00 | 19.05 | C |
| ATOM | 255 | C | THR | A | 38 | 17.589 | 13.750 | −8.220 | 1.00 | 18.54 | C |
| ATOM | 256 | O | THR | A | 38 | 18.716 | 13.357 | −7.942 | 1.00 | 19.06 | O |
| ATOM | 257 | N | VAL | A | 39 | 16.823 | 14.387 | −7.342 | 1.00 | 16.92 | N |
| ATOM | 258 | CA | VAL | A | 39 | 17.256 | 14.629 | −5.964 | 1.00 | 17.12 | C |
| ATOM | 259 | CB | VAL | A | 39 | 16.296 | 15.651 | −5.283 | 1.00 | 16.75 | C |
| ATOM | 260 | CG1 | VAL | A | 39 | 16.601 | 15.836 | −3.789 | 1.00 | 17.52 | C |
| ATOM | 261 | CG2 | VAL | A | 39 | 16.350 | 16.980 | −6.017 | 1.00 | 15.54 | C |
| ATOM | 262 | C | VAL | A | 39 | 17.315 | 13.338 | −5.148 | 1.00 | 17.46 | C |
| ATOM | 263 | O | VAL | A | 39 | 16.390 | 12.541 | −5.164 | 1.00 | 17.06 | O |
| ATOM | 264 | N | SER | A | 40 | 18.424 | 13.136 | −4.435 | 1.00 | 17.68 | N |
| ATOM | 265 | CA | SER | A | 40 | 18.441 | 12.193 | −3.315 | 1.00 | 18.58 | C |
| ATOM | 266 | CB | SER | A | 40 | 19.397 | 11.027 | −3.552 | 1.00 | 19.00 | C |
| ATOM | 267 | OG | SER | A | 40 | 20.692 | 11.505 | −3.797 | 1.00 | 21.47 | O |
| ATOM | 268 | C | SER | A | 40 | 18.843 | 12.987 | −2.075 | 1.00 | 17.60 | C |
| ATOM | 269 | O | SER | A | 40 | 19.448 | 14.046 | −2.209 | 1.00 | 18.01 | O |
| ATOM | 270 | N | ASN | A | 41 | 18.496 | 12.470 | −0.901 | 1.00 | 18.90 | N |
| ATOM | 271 | CA | ASN | A | 41 | 18.746 | 13.150 | 0.374 | 1.00 | 18.58 | C |
| ATOM | 272 | CB | ASN | A | 41 | 18.370 | 12.236 | 1.538 | 1.00 | 18.85 | C |
| ATOM | 273 | CG | ASN | A | 41 | 16.874 | 12.043 | 1.670 | 1.00 | 18.46 | C |
| ATOM | 274 | OD1 | ASN | A | 41 | 16.381 | 10.933 | 1.825 | 1.00 | 21.16 | O |
| ATOM | 275 | ND2 | ASN | A | 41 | 16.137 | 13.139 | 1.614 | 1.00 | 15.79 | N |
| ATOM | 276 | C | ASN | A | 41 | 20.177 | 13.638 | 0.535 | 1.00 | 19.52 | C |
| ATOM | 277 | O | ASN | A | 41 | 21.117 | 12.846 | 0.432 | 1.00 | 20.11 | O |
| ATOM | 278 | N | GLY | A | 42 | 20.342 | 14.941 | 0.763 | 1.00 | 19.59 | N |
| ATOM | 279 | CA | GLY | A | 42 | 21.663 | 15.520 | 1.022 | 1.00 | 20.73 | C |
| ATOM | 280 | C | GLY | A | 42 | 22.414 | 15.963 | −0.218 | 1.00 | 21.31 | C |
| ATOM | 281 | O | GLY | A | 42 | 23.460 | 16.626 | −0.121 | 1.00 | 22.32 | O |
| ATOM | 282 | N | LEU | A | 43 | 21.887 | 15.603 | −1.388 | 1.00 | 21.41 | N |
| ATOM | 283 | CA | LEU | A | 43 | 22.442 | 16.031 | −2.661 | 1.00 | 21.73 | C |
| ATOM | 284 | CB | LEU | A | 43 | 21.517 | 15.623 | −3.810 | 1.00 | 22.13 | C |
| ATOM | 285 | CG | LEU | A | 43 | 21.984 | 15.984 | −5.223 | 1.00 | 22.38 | C |
| ATOM | 286 | CD1 | LEU | A | 43 | 23.238 | 15.189 | −5.609 | 1.00 | 21.96 | C |
| ATOM | 287 | CD2 | LEU | A | 43 | 20.871 | 15.734 | −6.233 | 1.00 | 22.92 | C |
| ATOM | 288 | C | LEU | A | 43 | 22.646 | 17.538 | −2.690 | 1.00 | 21.61 | C |
| ATOM | 289 | O | LEU | A | 43 | 21.733 | 18.301 | −2.371 | 1.00 | 20.99 | O |
| ATOM | 290 | N | GLU | A | 44 | 23.844 | 17.978 | −3.044 | 1.00 | 21.67 | N |
| ATOM | 291 | CA | GLU | A | 44 | 24.071 | 19.398 | −3.182 | 1.00 | 21.58 | C |
| ATOM | 292 | CB | GLU | A | 44 | 25.510 | 19.795 | −2.838 | 1.00 | 22.31 | C |
| ATOM | 293 | CG | GLU | A | 44 | 25.767 | 21.264 | −3.143 | 1.00 | 23.85 | C |
| ATOM | 294 | CD | GLU | A | 44 | 26.898 | 21.869 | −2.338 | 1.00 | 26.59 | C |
| ATOM | 295 | OE1 | GLU | A | 44 | 27.689 | 21.117 | −1.717 | 1.00 | 28.58 | O |
| ATOM | 296 | OE2 | GLU | A | 44 | 26.989 | 23.108 | −2.334 | 1.00 | 27.92 | O |
| ATOM | 297 | C | GLU | A | 44 | 23.709 | 19.854 | −4.577 | 1.00 | 21.63 | C |
| ATOM | 298 | O | GLU | A | 44 | 24.173 | 19.290 | −5.574 | 1.00 | 21.73 | O |
| ATOM | 299 | N | LEU | A | 45 | 22.856 | 20.871 | −4.648 | 1.00 | 21.04 | N |
| ATOM | 300 | CA | LEU | A | 45 | 22.528 | 21.507 | −5.914 | 1.00 | 20.48 | C |
| ATOM | 301 | CB | LEU | A | 45 | 21.049 | 21.313 | −6.277 | 1.00 | 20.50 | C |
| ATOM | 302 | CG | LEU | A | 45 | 20.593 | 19.903 | −6.656 | 1.00 | 19.35 | C |
| ATOM | 303 | CD1 | LEU | A | 45 | 19.071 | 19.851 | −6.802 | 1.00 | 18.58 | C |
| ATOM | 304 | CD2 | LEU | A | 45 | 21.269 | 19.409 | −7.938 | 1.00 | 21.10 | C |
| ATOM | 305 | C | LEU | A | 45 | 22.853 | 22.981 | −5.857 | 1.00 | 20.71 | C |
| ATOM | 306 | O | LEU | A | 45 | 22.694 | 23.618 | −4.808 | 1.00 | 21.18 | O |
| ATOM | 307 | N | LYS | A | 46 | 23.343 | 23.520 | −6.970 | 1.00 | 20.32 | N |
| ATOM | 308 | CA | LYS | A | 46 | 23.616 | 24.949 | −7.055 | 1.00 | 20.19 | C |
| ATOM | 309 | CB | LYS | A | 46 | 24.571 | 25.284 | −8.209 | 1.00 | 21.02 | C |
| ATOM | 310 | CG | LYS | A | 46 | 25.895 | 24.535 | −8.156 | 1.00 | 23.71 | C |
| ATOM | 311 | CD | LYS | A | 46 | 26.979 | 25.263 | −8.955 | 1.00 | 28.47 | C |
| ATOM | 312 | CE | LYS | A | 46 | 26.486 | 25.721 | −10.324 | 1.00 | 30.79 | C |
| ATOM | 313 | NZ | LYS | A | 46 | 27.442 | 26.682 | −10.955 | 1.00 | 32.21 | N |
| ATOM | 314 | C | LYS | A | 46 | 22.309 | 25.705 | −7.222 | 1.00 | 19.43 | C |
| ATOM | 315 | O | LYS | A | 46 | 21.385 | 25.207 | −7.870 | 1.00 | 18.78 | O |
| ATOM | 316 | N | PRO | A | 47 | 22.219 | 26.915 | −6.648 | 1.00 | 19.03 | N |
| ATOM | 317 | CA | PRO | A | 47 | 21.036 | 27.749 | −6.838 | 1.00 | 19.24 | C |
| ATOM | 318 | CB | PRO | A | 47 | 21.468 | 29.098 | −6.242 | 1.00 | 19.42 | C |
| ATOM | 319 | CG | PRO | A | 47 | 22.429 | 28.721 | −5.192 | 1.00 | 19.39 | C |
| ATOM | 320 | CD | PRO | A | 47 | 23.201 | 27.563 | −5.759 | 1.00 | 19.56 | C |
| ATOM | 321 | C | PRO | A | 47 | 20.602 | 27.882 | −8.311 | 1.00 | 18.88 | C |
| ATOM | 322 | O | PRO | A | 47 | 19.403 | 27.843 | −8.608 | 1.00 | 18.80 | O |
| ATOM | 323 | N | SER | A | 48 | 21.553 | 28.041 | −9.235 | 1.00 | 19.58 | N |
| ATOM | 324 | CA | SER | A | 48 | 21.211 | 28.192 | −10.651 | 1.00 | 19.51 | C |
| ATOM | 325 | CB | SER | A | 48 | 22.468 | 28.474 | −11.486 | 1.00 | 20.07 | C |
| ATOM | 326 | OG | SER | A | 48 | 23.320 | 27.347 | −11.455 | 1.00 | 22.63 | O |
| ATOM | 327 | C | SER | A | 48 | 20.473 | 26.977 | −11.226 | 1.00 | 19.17 | C |
| ATOM | 328 | O | SER | A | 48 | 19.767 | 27.096 | −12.216 | 1.00 | 19.62 | O |
| ATOM | 329 | N | MET | A | 49 | 20.653 | 25.812 | −10.617 | 1.00 | 18.45 | N |
| ATOM | 330 | CA | MET | A | 49 | 19.988 | 24.603 | −11.080 | 1.00 | 18.55 | C |
| ATOM | 331 | CB | MET | A | 49 | 20.745 | 23.363 | −10.589 | 1.00 | 18.68 | C |
| ATOM | 332 | CG | MET | A | 49 | 22.172 | 23.209 | −11.148 | 1.00 | 20.25 | C |
| ATOM | 333 | SD | MET | A | 49 | 23.138 | 22.099 | −10.126 | 1.00 | 23.81 | S |
| ATOM | 334 | CE | MET | A | 49 | 24.770 | 22.202 | −10.862 | 1.00 | 22.41 | C |
| ATOM | 335 | C | MET | A | 49 | 18.523 | 24.533 | −10.645 | 1.00 | 17.01 | C |
| ATOM | 336 | O | MET | A | 49 | 17.742 | 23.780 | −11.216 | 1.00 | 16.05 | O |
| ATOM | 337 | N | VAL | A | 50 | 18.156 | 25.331 | −9.644 | 1.00 | 14.99 | N |
| ATOM | 338 | CA | VAL | A | 50 | 16.839 | 25.205 | −8.993 | 1.00 | 13.94 | C |
| ATOM | 339 | CB | VAL | A | 50 | 16.974 | 24.654 | −7.542 | 1.00 | 12.87 | C |
| ATOM | 340 | CG1 | VAL | A | 50 | 17.667 | 23.304 | −7.565 | 1.00 | 13.50 | C |
| ATOM | 341 | CG2 | VAL | A | 50 | 17.724 | 25.622 | −6.631 | 1.00 | 14.15 | C |
| ATOM | 342 | C | VAL | A | 50 | 15.977 | 26.468 | −9.071 | 1.00 | 12.86 | C |
| ATOM | 343 | O | VAL | A | 50 | 15.020 | 26.634 | −8.300 | 1.00 | 12.24 | O |
| ATOM | 344 | N | THR | A | 51 | 16.289 | 27.345 | −10.018 | 1.00 | 13.48 | N |
| ATOM | 345 | CA | THR | A | 51 | 15.551 | 28.596 | −10.204 | 1.00 | 13.59 | C |
| ATOM | 346 | CB | THR | A | 51 | 16.220 | 29.446 | −11.302 | 1.00 | 14.31 | C |
| ATOM | 347 | OG1 | THR | A | 51 | 16.404 | 28.628 | −12.463 | 1.00 | 16.31 | O |
| ATOM | 348 | CG2 | THR | A | 51 | 17.574 | 29.940 | −10.845 | 1.00 | 14.50 | C |
| ATOM | 349 | C | THR | A | 51 | 14.060 | 28.386 | −10.544 | 1.00 | 13.98 | C |
| ATOM | 350 | O | THR | A | 51 | 13.204 | 29.184 | −10.159 | 1.00 | 14.64 | O |
| ATOM | 351 | N | HIS | A | 52 | 13.754 | 27.313 | −11.276 | 1.00 | 13.33 | N |
| ATOM | 352 | CA | HIS | A | 52 | 12.386 | 26.982 | −11.647 | 1.00 | 13.04 | C |
| ATOM | 353 | CB | HIS | A | 52 | 12.282 | 26.832 | −13.151 | 1.00 | 13.68 | C |
| ATOM | 354 | CG | HIS | A | 52 | 12.477 | 28.114 | −13.884 | 1.00 | 14.46 | C |
| ATOM | 355 | ND1 | HIS | A | 52 | 13.708 | 28.721 | −14.014 | 1.00 | 16.14 | N |
| ATOM | 356 | CE1 | HIS | A | 52 | 13.565 | 29.843 | −14.699 | 1.00 | 16.08 | C |
| ATOM | 357 | NE2 | HIS | A | 52 | 12.291 | 29.983 | −15.011 | 1.00 | 18.18 | N |
| ATOM | 358 | CD2 | HIS | A | 52 | 11.588 | 28.920 | −14.500 | 1.00 | 15.40 | C |
| ATOM | 359 | C | HIS | A | 52 | 11.971 | 25.678 | −10.991 | 1.00 | 11.09 | C |
| ATOM | 360 | O | HIS | A | 52 | 12.821 | 24.887 | −10.577 | 1.00 | 11.88 | O |
| ATOM | 361 | N | GLN | A | 53 | 10.662 | 25.453 | −10.916 | 1.00 | 11.62 | N |
| ATOM | 362 | CA | GLN | A | 53 | 10.182 | 24.293 | −10.182 | 1.00 | 11.19 | C |
| ATOM | 363 | CB | GLN | A | 53 | 8.651 | 24.285 | −10.044 | 1.00 | 10.34 | C |
| ATOM | 364 | CG | GLN | A | 53 | 7.900 | 24.105 | −11.336 | 1.00 | 11.51 | C |
| ATOM | 365 | CD | GLN | A | 53 | 6.494 | 24.537 | −11.186 | 1.00 | 15.07 | C |
| ATOM | 366 | OE1 | GLN | A | 53 | 6.220 | 25.739 | −11.055 | 1.00 | 15.59 | O |
| ATOM | 367 | NE2 | GLN | A | 53 | 5.583 | 23.584 | −11.193 | 1.00 | 14.39 | N |
| ATOM | 368 | C | GLN | A | 53 | 10.667 | 22.995 | −10.820 | 1.00 | 10.53 | C |
| ATOM | 369 | O | GLN | A | 53 | 10.759 | 22.893 | −12.064 | 1.00 | 11.42 | O |
| ATOM | 370 | N | PRO | A | 54 | 10.977 | 21.992 | −9.983 | 1.00 | 10.57 | N |
| ATOM | 371 | CA | PRO | A | 54 | 11.353 | 20.695 | −10.519 | 1.00 | 10.47 | C |
| ATOM | 372 | CB | PRO | A | 54 | 11.933 | 19.974 | −9.312 | 1.00 | 10.85 | C |
| ATOM | 373 | CG | PRO | A | 54 | 11.188 | 20.546 | −8.128 | 1.00 | 10.74 | C |
| ATOM | 374 | CD | PRO | A | 54 | 10.947 | 22.001 | −8.499 | 1.00 | 9.97 | C |
| ATOM | 375 | C | PRO | A | 54 | 10.128 | 19.932 | −10.989 | 1.00 | 11.10 | C |
| ATOM | 376 | O | PRO | A | 54 | 8.977 | 20.289 | −10.664 | 1.00 | 11.18 | O |
| ATOM | 377 | N | ARG | A | 55 | 10.362 | 18.893 | −11.779 | 1.00 | 11.44 | N |
| ATOM | 378 | CA | ARG | A | 55 | 9.305 | 17.929 | −12.042 | 1.00 | 12.31 | C |
| ATOM | 379 | CB | ARG | A | 55 | 9.648 | 17.104 | −13.267 | 1.00 | 13.37 | C |
| ATOM | 380 | CG | ARG | A | 55 | 9.553 | 17.863 | −14.555 | 1.00 | 16.91 | C |
| ATOM | 381 | CD | ARG | A | 55 | 9.591 | 16.900 | −15.732 | 1.00 | 19.52 | C |
| ATOM | 382 | NE | ARG | A | 55 | 10.788 | 16.061 | −15.678 | 1.00 | 21.39 | N |
| ATOM | 383 | CZ | ARG | A | 55 | 12.021 | 16.508 | −15.883 | 1.00 | 20.16 | C |
| ATOM | 384 | NH1 | ARG | A | 55 | 12.238 | 17.792 | −16.157 | 1.00 | 22.79 | N |
| ATOM | 385 | NH2 | ARG | A | 55 | 13.049 | 15.669 | −15.806 | 1.00 | 21.29 | N |
| ATOM | 386 | C | ARG | A | 55 | 9.191 | 17.027 | −10.841 | 1.00 | 12.50 | C |
| ATOM | 387 | O | ARG | A | 55 | 10.201 | 16.551 | −10.328 | 1.00 | 12.92 | O |
| ATOM | 388 | N | VAL | A | 56 | 7.978 | 16.792 | −10.366 | 1.00 | 11.65 | N |
| ATOM | 389 | CA | VAL | A | 56 | 7.798 | 15.967 | −9.178 | 1.00 | 11.93 | C |
| ATOM | 390 | CB | VAL | A | 56 | 7.371 | 16.797 | −7.952 | 1.00 | 12.02 | C |
| ATOM | 391 | CG1 | VAL | A | 56 | 7.209 | 15.940 | −6.723 | 1.00 | 12.50 | C |
| ATOM | 392 | CG2 | VAL | A | 56 | 8.393 | 17.899 | −7.678 | 1.00 | 11.26 | C |
| ATOM | 393 | C | VAL | A | 56 | 6.797 | 14.888 | −9.487 | 1.00 | 13.55 | C |
| ATOM | 394 | O | VAL | A | 56 | 5.595 | 15.142 | −9.510 | 1.00 | 13.06 | O |
| ATOM | 395 | N | GLU | A | 57 | 7.319 | 13.690 | −9.734 | 1.00 | 13.98 | N |
| ATOM | 396 | CA | GLU | A | 57 | 6.489 | 12.547 | −10.035 | 1.00 | 15.76 | C |
| ATOM | 397 | CB | GLU | A | 57 | 7.231 | 11.563 | −10.946 | 1.00 | 15.61 | C |
| ATOM | 398 | CG | GLU | A | 57 | 7.449 | 12.137 | −12.326 | 1.00 | 16.98 | C |
| ATOM | 399 | CD | GLU | A | 57 | 8.192 | 11.204 | −13.291 | 1.00 | 19.08 | C |
| ATOM | 400 | OE1 | GLU | A | 57 | 8.359 | 9.998 | −12.990 | 1.00 | 24.85 | O |
| ATOM | 401 | OE2 | GLU | A | 57 | 8.589 | 11.703 | −14.370 | 1.00 | 25.69 | O |
| ATOM | 402 | C | GLU | A | 57 | 6.079 | 11.914 | −8.737 | 1.00 | 15.53 | C |
| ATOM | 403 | O | GLU | A | 57 | 6.872 | 11.803 | −7.795 | 1.00 | 15.86 | O |
| ATOM | 404 | N | VAL | A | 58 | 4.804 | 11.548 | −8.666 | 1.00 | 15.80 | N |
| ATOM | 405 | CA | VAL | A | 58 | 4.287 | 10.872 | −7.488 | 1.00 | 17.11 | C |
| ATOM | 406 | CB | VAL | A | 58 | 3.144 | 11.638 | −6.767 | 1.00 | 17.67 | C |
| ATOM | 407 | CG1 | VAL | A | 58 | 3.577 | 13.081 | −6.451 | 1.00 | 16.49 | C |
| ATOM | 408 | CG2 | VAL | A | 58 | 1.843 | 11.613 | −7.563 | 1.00 | 19.47 | C |
| ATOM | 409 | C | VAL | A | 58 | 3.846 | 9.466 | −7.863 | 1.00 | 17.01 | C |
| ATOM | 410 | O | VAL | A | 58 | 3.220 | 9.241 | −8.923 | 1.00 | 17.90 | O |
| ATOM | 411 | N | GLY | A | 59 | 4.182 | 8.529 | −6.994 | 1.00 | 16.97 | N |
| ATOM | 412 | CA | GLY | A | 59 | 3.787 | 7.144 | −7.179 | 1.00 | 17.19 | C |
| ATOM | 413 | C | GLY | A | 59 | 2.376 | 6.870 | −6.695 | 1.00 | 17.33 | C |
| ATOM | 414 | O | GLY | A | 59 | 1.438 | 7.647 | −6.932 | 1.00 | 16.56 | O |
| ATOM | 415 | N | GLY | A | 60 | 2.224 | 5.744 | −6.008 | 1.00 | 18.31 | N |
| ATOM | 416 | CA | GLY | A | 60 | 0.909 | 5.284 | −5.586 | 1.00 | 19.22 | C |
| ATOM | 417 | C | GLY | A | 60 | 0.259 | 4.294 | −6.557 | 1.00 | 20.17 | C |
| ATOM | 418 | O | GLY | A | 60 | 0.885 | 3.886 | −7.546 | 1.00 | 20.71 | O |
| ATOM | 419 | N | ASN | A | 61 | −0.993 | 3.931 | −6.264 | 1.00 | 20.95 | N |
| ATOM | 420 | CA | ASN | A | 61 | −1.725 | 2.864 | −6.968 | 1.00 | 22.07 | C |
| ATOM | 421 | CB | ASN | A | 61 | −2.785 | 2.245 | −6.045 | 1.00 | 22.28 | C |
| ATOM | 422 | CG | ASN | A | 61 | −2.244 | 1.112 | −5.205 | 1.00 | 25.63 | C |
| ATOM | 423 | OD1 | ASN | A | 61 | −1.524 | 0.248 | −5.695 | 1.00 | 28.95 | O |
| ATOM | 424 | ND2 | ASN | A | 61 | −2.612 | 1.096 | −3.935 | 1.00 | 28.97 | N |
| ATOM | 425 | C | ASN | A | 61 | −2.435 | 3.335 | −8.218 | 1.00 | 21.66 | C |
| ATOM | 426 | O | ASN | A | 61 | −2.480 | 2.617 | −9.221 | 1.00 | 22.35 | O |
| ATOM | 427 | N | ASP | A | 62 | −3.044 | 4.515 | −8.137 | 1.00 | 21.12 | N |
| ATOM | 428 | CA | ASP | A | 62 | −3.845 | 5.041 | −9.237 | 1.00 | 21.14 | C |
| ATOM | 429 | CB | ASP | A | 62 | −5.113 | 4.184 | −9.483 | 1.00 | 21.61 | C |
| ATOM | 430 | CG | ASP | A | 62 | −6.047 | 4.134 | −8.274 | 1.00 | 22.61 | C |
| ATOM | 431 | OD1 | ASP | A | 62 | −6.580 | 5.188 | −7.876 | 1.00 | 24.91 | O |
| ATOM | 432 | OD2 | ASP | A | 62 | −6.287 | 3.023 | −7.746 | 1.00 | 24.60 | O |
| ATOM | 433 | C | ASP | A | 62 | −4.198 | 6.506 | −9.049 | 1.00 | 20.60 | C |
| ATOM | 434 | O | ASP | A | 62 | −3.890 | 7.115 | −8.023 | 1.00 | 19.85 | O |
| ATOM | 435 | N | MET | A | 63 | −4.862 | 7.052 | −10.061 | 1.00 | 19.74 | N |
| ATOM | 436 | CA | MET | A | 63 | −5.155 | 8.482 | −10.132 | 1.00 | 19.71 | C |
| ATOM | 437 | CB | MET | A | 63 | −5.462 | 8.876 | −11.578 | 1.00 | 19.87 | C |
| ATOM | 438 | CG | MET | A | 63 | −4.252 | 8.824 | −12.474 | 1.00 | 19.72 | C |
| ATOM | 439 | SD | MET | A | 63 | −4.641 | 9.187 | −14.196 | 1.00 | 23.34 | S |
| ATOM | 440 | CE | MET | A | 63 | −5.239 | 10.851 | −14.073 | 1.00 | 23.23 | C |
| ATOM | 441 | C | MET | A | 63 | −6.279 | 8.949 | −9.219 | 1.00 | 18.85 | C |
| ATOM | 442 | O | MET | A | 63 | −6.583 | 10.142 | −9.183 | 1.00 | 18.07 | O |
| ATOM | 443 | N | ARG | A | 64 | −6.908 | 8.023 | −8.496 | 1.00 | 18.97 | N |
| ATOM | 444 | CA | ARG | A | 64 | −8.016 | 8.387 | −7.616 | 1.00 | 18.87 | C |
| ATOM | 445 | CB | ARG | A | 64 | −9.180 | 7.387 | −7.736 | 1.00 | 19.18 | C |
| ATOM | 446 | CG | ARG | A | 64 | −9.836 | 7.376 | −9.104 | 1.00 | 21.09 | C |
| ATOM | 447 | CD | ARG | A | 64 | −11.063 | 6.463 | −9.113 | 1.00 | 21.95 | C |
| ATOM | 448 | NE | ARG | A | 64 | −12.268 | 7.086 | −8.552 | 1.00 | 26.78 | N |
| ATOM | 449 | CZ | ARG | A | 64 | −12.827 | 6.756 | −7.387 | 1.00 | 28.44 | C |
| ATOM | 450 | NH1 | ARG | A | 64 | −12.300 | 5.797 | −6.628 | 1.00 | 29.72 | N |
| ATOM | 451 | NH2 | ARG | A | 64 | −13.930 | 7.383 | −6.977 | 1.00 | 30.04 | N |
| ATOM | 452 | C | ARG | A | 64 | −7.566 | 8.560 | −6.164 | 1.00 | 18.10 | C |
| ATOM | 453 | O | ARG | A | 64 | −8.379 | 8.828 | −5.282 | 1.00 | 17.60 | O |
| ATOM | 454 | N | THR | A | 65 | −6.265 | 8.402 | −5.928 | 1.00 | 16.62 | N |
| ATOM | 455 | CA | THR | A | 65 | −5.654 | 8.824 | −4.671 | 1.00 | 16.22 | C |
| ATOM | 456 | CB | THR | A | 65 | −4.607 | 7.817 | −4.152 | 1.00 | 16.94 | C |
| ATOM | 457 | OG1 | THR | A | 65 | −5.246 | 6.567 | −3.857 | 1.00 | 19.49 | O |
| ATOM | 458 | CG2 | THR | A | 65 | −3.955 | 8.323 | −2.872 | 1.00 | 18.53 | C |
| ATOM | 459 | C | THR | A | 65 | −4.985 | 10.164 | −4.948 | 1.00 | 14.81 | C |
| ATOM | 460 | O | THR | A | 65 | −4.329 | 10.331 | −5.994 | 1.00 | 15.24 | O |
| ATOM | 461 | N | PHE | A | 66 | −5.157 | 11.106 | −4.021 | 1.00 | 13.11 | N |
| ATOM | 462 | CA | PHE | A | 66 | −4.630 | 12.459 | −4.197 | 1.00 | 12.59 | C |
| ATOM | 463 | CB | PHE | A | 66 | −5.768 | 13.486 | −4.208 | 1.00 | 13.15 | C |
| ATOM | 464 | CG | PHE | A | 66 | −6.740 | 13.272 | −5.318 | 1.00 | 14.50 | C |
| ATOM | 465 | CD1 | PHE | A | 66 | −6.507 | 13.820 | −6.573 | 1.00 | 14.94 | C |
| ATOM | 466 | CE1 | PHE | A | 66 | −7.398 | 13.585 | −7.635 | 1.00 | 17.78 | C |
| ATOM | 467 | CZ | PHE | A | 66 | −8.527 | 12.785 | −7.424 | 1.00 | 17.85 | C |
| ATOM | 468 | CE2 | PHE | A | 66 | −8.760 | 12.220 | −6.174 | 1.00 | 18.68 | C |
| ATOM | 469 | CD2 | PHE | A | 66 | −7.855 | 12.454 | −5.126 | 1.00 | 16.22 | C |
| ATOM | 470 | C | PHE | A | 66 | −3.599 | 12.811 | −3.143 | 1.00 | 12.46 | C |
| ATOM | 471 | O | PHE | A | 66 | −3.707 | 12.405 | −1.987 | 1.00 | 12.11 | O |
| ATOM | 472 | N | TYR | A | 67 | −2.626 | 13.621 | −3.564 | 1.00 | 11.47 | N |
| ATOM | 473 | CA | TYR | A | 67 | −1.486 | 13.976 | −2.740 | 1.00 | 10.69 | C |
| ATOM | 474 | CB | TYR | A | 67 | −0.231 | 13.287 | −3.283 | 1.00 | 11.54 | C |
| ATOM | 475 | CG | TYR | A | 67 | −0.299 | 11.783 | −3.258 | 1.00 | 12.17 | C |
| ATOM | 476 | CD1 | TYR | A | 67 | −0.789 | 11.084 | −4.356 | 1.00 | 12.88 | C |
| ATOM | 477 | CE1 | TYR | A | 67 | −0.863 | 9.696 | −4.344 | 1.00 | 15.49 | C |
| ATOM | 478 | CZ | TYR | A | 67 | −0.467 | 9.007 | −3.229 | 1.00 | 14.40 | C |
| ATOM | 479 | OH | TYR | A | 67 | −0.544 | 7.623 | −3.252 | 1.00 | 16.14 | O |
| ATOM | 480 | CE2 | TYR | A | 67 | 0.030 | 9.669 | −2.108 | 1.00 | 13.73 | C |
| ATOM | 481 | CD2 | TYR | A | 67 | 0.096 | 11.058 | −2.127 | 1.00 | 12.45 | C |
| ATOM | 482 | C | TYR | A | 67 | −1.259 | 15.485 | −2.645 | 1.00 | 10.61 | C |
| ATOM | 483 | O | TYR | A | 67 | −1.557 | 16.247 | −3.578 | 1.00 | 10.82 | O |
| ATOM | 484 | N | THR | A | 68 | −0.704 | 15.890 | −1.505 | 1.00 | 10.52 | N |
| ATOM | 485 | CA | THR | A | 68 | −0.263 | 17.277 | −1.282 | 1.00 | 10.36 | C |
| ATOM | 486 | CB | THR | A | 68 | −0.908 | 17.865 | −0.022 | 1.00 | 10.43 | C |
| ATOM | 487 | OG1 | THR | A | 68 | −2.267 | 18.195 | −0.329 | 1.00 | 10.77 | O |
| ATOM | 488 | CG2 | THR | A | 68 | −0.187 | 19.143 | 0.444 | 1.00 | 11.32 | C |
| ATOM | 489 | C | THR | A | 68 | 1.253 | 17.302 | −1.166 | 1.00 | 9.84 | C |
| ATOM | 490 | O | THR | A | 68 | 1.842 | 16.490 | −0.463 | 1.00 | 10.24 | O |
| ATOM | 491 | N | LEU | A | 69 | 1.865 | 18.253 | −1.877 | 1.00 | 9.28 | N |
| ATOM | 492 | CA | LEU | A | 69 | 3.304 | 18.502 | −1.870 | 1.00 | 8.99 | C |
| ATOM | 493 | CB | LEU | A | 69 | 3.862 | 18.468 | −3.294 | 1.00 | 9.04 | C |
| ATOM | 494 | CG | LEU | A | 69 | 5.358 | 18.743 | −3.419 | 1.00 | 10.30 | C |
| ATOM | 495 | CD1 | LEU | A | 69 | 6.159 | 17.556 | −2.945 | 1.00 | 11.75 | C |
| ATOM | 496 | CD2 | LEU | A | 69 | 5.683 | 19.017 | −4.870 | 1.00 | 10.37 | C |
| ATOM | 497 | C | LEU | A | 69 | 3.605 | 19.862 | −1.244 | 1.00 | 8.91 | C |
| ATOM | 498 | O | LEU | A | 69 | 3.053 | 20.894 | −1.650 | 1.00 | 9.05 | O |
| ATOM | 499 | N | VAL | A | 70 | 4.492 | 19.828 | −0.254 | 1.00 | 8.68 | N |
| ATOM | 500 | CA | VAL | A | 70 | 4.982 | 20.994 | 0.472 | 1.00 | 8.66 | C |
| ATOM | 501 | CB | VAL | A | 70 | 4.811 | 20.786 | 1.982 | 1.00 | 8.76 | C |
| ATOM | 502 | CG1 | VAL | A | 70 | 5.428 | 21.967 | 2.765 | 1.00 | 11.73 | C |
| ATOM | 503 | CG2 | VAL | A | 70 | 3.345 | 20.583 | 2.320 | 1.00 | 11.81 | C |
| ATOM | 504 | C | VAL | A | 70 | 6.464 | 21.117 | 0.207 | 1.00 | 7.88 | C |
| ATOM | 505 | O | VAL | A | 70 | 7.188 | 20.135 | 0.314 | 1.00 | 8.44 | O |
| ATOM | 506 | N | MET | A | 71 | 6.935 | 22.326 | −0.079 | 1.00 | 8.16 | N |
| ATOM | 507 | CA | MET | A | 71 | 8.375 | 22.575 | −0.162 | 1.00 | 7.63 | C |
| ATOM | 508 | CB | MET | A | 71 | 8.801 | 22.859 | −1.605 | 1.00 | 7.87 | C |
| ATOM | 509 | CG | MET | A | 71 | 10.298 | 23.096 | −1.763 | 1.00 | 8.73 | C |
| ATOM | 510 | SD | MET | A | 71 | 10.624 | 23.912 | −3.339 | 1.00 | 8.90 | S |
| ATOM | 511 | CE | MET | A | 71 | 12.412 | 24.075 | −3.272 | 1.00 | 10.72 | C |
| ATOM | 512 | C | MET | A | 71 | 8.737 | 23.719 | 0.761 | 1.00 | 6.96 | C |
| ATOM | 513 | O | MET | A | 71 | 8.220 | 24.833 | 0.606 | 1.00 | 7.60 | O |
| ATOM | 514 | N | VAL | A | 72 | 9.636 | 23.441 | 1.714 | 1.00 | 7.01 | N |
| ATOM | 515 | CA | VAL | A | 72 | 10.044 | 24.447 | 2.711 | 1.00 | 7.91 | C |
| ATOM | 516 | CB | VAL | A | 72 | 9.396 | 24.178 | 4.105 | 1.00 | 7.93 | C |
| ATOM | 517 | CG1 | VAL | A | 72 | 7.883 | 24.346 | 4.040 | 1.00 | 10.28 | C |
| ATOM | 518 | CG2 | VAL | A | 72 | 9.742 | 22.769 | 4.625 | 1.00 | 9.84 | C |
| ATOM | 519 | C | VAL | A | 72 | 11.557 | 24.552 | 2.884 | 1.00 | 8.26 | C |
| ATOM | 520 | O | VAL | A | 72 | 12.276 | 23.586 | 2.628 | 1.00 | 8.13 | O |
| ATOM | 521 | N | ASP | A | 73 | 12.011 | 25.743 | 3.298 | 1.00 | 8.24 | N |
| ATOM | 522 | CA | ASP | A | 73 | 13.401 | 25.974 | 3.737 | 1.00 | 8.48 | C |
| ATOM | 523 | CB | ASP | A | 73 | 14.015 | 27.175 | 3.029 | 1.00 | 9.04 | C |
| ATOM | 524 | CG | ASP | A | 73 | 15.486 | 27.391 | 3.379 | 1.00 | 10.14 | C |
| ATOM | 525 | OD1 | ASP | A | 73 | 16.011 | 26.721 | 4.280 | 1.00 | 9.98 | O |
| ATOM | 526 | OD2 | ASP | A | 73 | 16.088 | 28.275 | 2.747 | 1.00 | 12.13 | O |
| ATOM | 527 | C | ASP | A | 73 | 13.346 | 26.211 | 5.247 | 1.00 | 8.65 | C |
| ATOM | 528 | O | ASP | A | 73 | 12.906 | 27.252 | 5.703 | 1.00 | 9.12 | O |
| ATOM | 529 | N | PRO | A | 74 | 13.782 | 25.226 | 6.033 | 1.00 | 9.39 | N |
| ATOM | 530 | CA | PRO | A | 74 | 13.805 | 25.409 | 7.489 | 1.00 | 9.78 | C |
| ATOM | 531 | CB | PRO | A | 74 | 13.891 | 23.978 | 7.999 | 1.00 | 10.36 | C |
| ATOM | 532 | CG | PRO | A | 74 | 14.702 | 23.280 | 6.961 | 1.00 | 9.48 | C |
| ATOM | 533 | CD | PRO | A | 74 | 14.228 | 23.869 | 5.642 | 1.00 | 9.67 | C |
| ATOM | 534 | C | PRO | A | 74 | 15.026 | 26.203 | 7.961 | 1.00 | 10.88 | C |
| ATOM | 535 | O | PRO | A | 74 | 15.128 | 26.513 | 9.155 | 1.00 | 11.53 | O |
| ATOM | 536 | N | ASP | A | 75 | 15.911 | 26.550 | 7.024 | 1.00 | 11.03 | N |
| ATOM | 537 | CA | ASP | A | 75 | 17.165 | 27.260 | 7.311 | 1.00 | 11.09 | C |
| ATOM | 538 | CB | ASP | A | 75 | 18.338 | 26.511 | 6.659 | 1.00 | 11.39 | C |
| ATOM | 539 | CG | ASP | A | 75 | 18.491 | 25.119 | 7.195 | 1.00 | 11.34 | C |
| ATOM | 540 | OD1 | ASP | A | 75 | 18.436 | 24.972 | 8.420 | 1.00 | 13.96 | O |
| ATOM | 541 | OD2 | ASP | A | 75 | 18.635 | 24.129 | 6.430 | 1.00 | 13.93 | O |
| ATOM | 542 | C | ASP | A | 75 | 17.140 | 28.703 | 6.830 | 1.00 | 11.19 | C |
| ATOM | 543 | O | ASP | A | 75 | 18.185 | 29.242 | 6.482 | 1.00 | 12.38 | O |
| ATOM | 544 | N | ALA | A | 76 | 15.955 | 29.316 | 6.793 | 1.00 | 11.30 | N |
| ATOM | 545 | CA | ALA | A | 76 | 15.777 | 30.669 | 6.274 | 1.00 | 12.10 | C |
| ATOM | 546 | CB | ALA | A | 76 | 14.511 | 30.740 | 5.405 | 1.00 | 12.47 | C |
| ATOM | 547 | C | ALA | A | 76 | 15.685 | 31.690 | 7.412 | 1.00 | 12.33 | C |
| ATOM | 548 | O | ALA | A | 76 | 14.973 | 31.464 | 8.389 | 1.00 | 12.27 | O |
| ATOM | 549 | N | PRO | A | 77 | 16.405 | 32.821 | 7.300 | 1.00 | 13.41 | N |
| ATOM | 550 | CA | PRO | A | 77 | 17.378 | 33.201 | 6.264 | 1.00 | 13.79 | C |
| ATOM | 551 | CB | PRO | A | 77 | 17.556 | 34.718 | 6.500 | 1.00 | 14.09 | C |
| ATOM | 552 | CG | PRO | A | 77 | 16.417 | 35.130 | 7.423 | 1.00 | 15.20 | C |
| ATOM | 553 | CD | PRO | A | 77 | 16.202 | 33.910 | 8.271 | 1.00 | 13.53 | C |
| ATOM | 554 | C | PRO | A | 77 | 18.739 | 32.480 | 6.323 | 1.00 | 14.46 | C |
| ATOM | 555 | O | PRO | A | 77 | 19.449 | 32.445 | 5.318 | 1.00 | 14.37 | O |
| ATOM | 556 | N | SER | A | 78 | 19.100 | 31.917 | 7.474 | 1.00 | 14.30 | N |
| ATOM | 557 | CA | SER | A | 78 | 20.381 | 31.226 | 7.646 | 1.00 | 15.55 | C |
| ATOM | 558 | CB | SEA | A | 78 | 21.394 | 32.166 | 8.307 | 1.00 | 16.01 | C |
| ATOM | 559 | OG | SEA | A | 78 | 22.591 | 31.496 | 8.666 | 1.00 | 20.32 | O |
| ATOM | 560 | C | SEA | A | 78 | 20.214 | 30.012 | 8.532 | 1.00 | 14.81 | C |
| ATOM | 561 | O | SEA | A | 78 | 19.428 | 30.050 | 9.477 | 1.00 | 13.88 | O |
| ATOM | 562 | N | PRO | A | 79 | 20.981 | 28.931 | 8.277 | 1.00 | 15.25 | N |
| ATOM | 563 | CA | PRO | A | 79 | 20.892 | 27.817 | 9.219 | 1.00 | 15.73 | C |
| ATOM | 564 | CB | PRO | A | 79 | 21.821 | 26.753 | 8.620 | 1.00 | 15.83 | C |
| ATOM | 565 | CG | PRO | A | 79 | 22.718 | 27.490 | 7.683 | 1.00 | 15.98 | C |
| ATOM | 566 | CD | PRO | A | 79 | 21.911 | 28.648 | 7.166 | 1.00 | 15.07 | C |
| ATOM | 567 | C | PRO | A | 79 | 21.312 | 28.196 | 10.646 | 1.00 | 15.88 | C |
| ATOM | 568 | O | PRO | A | 79 | 20.826 | 27.608 | 11.601 | 1.00 | 16.58 | O |
| ATOM | 569 | N | SER | A | 80 | 22.175 | 29.197 | 10.791 | 1.00 | 16.07 | N |
| ATOM | 570 | CA | SER | A | 80 | 22.585 | 29.631 | 12.140 | 1.00 | 16.19 | C |
| ATOM | 571 | CB | SER | A | 80 | 24.035 | 30.105 | 12.116 | 1.00 | 17.04 | C |
| ATOM | 572 | OG | SEA | A | 80 | 24.132 | 31.288 | 11.370 | 1.00 | 17.95 | O |
| ATOM | 573 | C | SEA | A | 80 | 21.676 | 30.713 | 12.746 | 1.00 | 16.20 | C |
| ATOM | 574 | O | SER | A | 80 | 21.848 | 31.107 | 13.900 | 1.00 | 15.71 | O |
| ATOM | 575 | N | ASP | A | 81 | 20.691 | 31.170 | 11.978 | 1.00 | 15.78 | N |
| ATOM | 576 | CA | ASP | A | 81 | 19.703 | 32.137 | 12.445 | 1.00 | 15.53 | C |
| ATOM | 577 | CB | ASP | A | 81 | 20.245 | 33.562 | 12.278 | 1.00 | 17.12 | C |
| ATOM | 578 | CG | ASP | A | 81 | 19.265 | 34.646 | 12.725 | 1.00 | 19.91 | C |
| ATOM | 579 | OD1 | ASP | A | 81 | 18.211 | 34.363 | 13.338 | 1.00 | 20.54 | O |
| ATOM | 580 | OD2 | ASP | A | 81 | 19.575 | 35.820 | 12.444 | 1.00 | 26.03 | O |
| ATOM | 581 | C | ASP | A | 81 | 18.409 | 31.911 | 11.633 | 1.00 | 14.33 | C |
| ATOM | 582 | O | ASP | A | 81 | 18.048 | 32.710 | 10.775 | 1.00 | 14.61 | O |
| ATOM | 583 | N | PRO | A | 82 | 17.725 | 30.791 | 11.893 | 1.00 | 13.32 | N |
| ATOM | 584 | CA | PRO | A | 82 | 16.580 | 30.414 | 11.055 | 1.00 | 12.43 | C |
| ATOM | 585 | CB | PRO | A | 82 | 16.513 | 28.895 | 11.247 | 1.00 | 12.84 | C |
| ATOM | 586 | CG | PRO | A | 82 | 16.989 | 28.687 | 12.670 | 1.00 | 12.18 | C |
| ATOM | 587 | CD | PRO | A | 82 | 18.021 | 29.771 | 12.922 | 1.00 | 13.84 | C |
| ATOM | 588 | C | PRO | A | 82 | 15.273 | 31.072 | 11.490 | 1.00 | 12.14 | C |
| ATOM | 589 | O | PRO | A | 82 | 14.271 | 30.393 | 11.728 | 1.00 | 11.56 | O |
| ATOM | 590 | N | ASN | A | 83 | 15.272 | 32.401 | 11.571 | 1.00 | 11.82 | N |
| ATOM | 591 | CA | ASN | A | 83 | 14.128 | 33.108 | 12.156 | 1.00 | 12.00 | C |
| ATOM | 592 | CB | ASN | A | 83 | 14.508 | 34.544 | 12.560 | 1.00 | 12.64 | C |
| ATOM | 593 | CG | ASN | A | 83 | 14.971 | 35.375 | 11.398 | 1.00 | 13.45 | C |
| ATOM | 594 | OD1 | ASN | A | 83 | 14.177 | 35.749 | 10.530 | 1.00 | 15.06 | O |
| ATOM | 595 | ND2 | ASN | A | 83 | 16.273 | 35.686 | 11.365 | 1.00 | 14.12 | N |
| ATOM | 596 | C | ASN | A | 83 | 12.844 | 33.101 | 11.322 | 1.00 | 12.24 | C |
| ATOM | 597 | O | ASN | A | 83 | 11.772 | 33.445 | 11.831 | 1.00 | 12.21 | O |
| ATOM | 598 | N | LED | A | 84 | 12.963 | 32.688 | 10.062 | 1.00 | 11.14 | N |
| ATOM | 599 | CA | LED | A | 84 | 11.785 | 32.538 | 9.199 | 1.00 | 10.21 | C |
| ATOM | 600 | CB | LEU | A | 84 | 12.025 | 33.158 | 7.823 | 1.00 | 11.50 | C |
| ATOM | 601 | CG | LEU | A | 84 | 12.373 | 34.657 | 7.770 | 1.00 | 11.33 | C |
| ATOM | 602 | CD1 | LEU | A | 84 | 12.589 | 35.063 | 6.327 | 1.00 | 15.59 | C |
| ATOM | 603 | CD2 | LEU | A | 84 | 11.297 | 35.497 | 8.466 | 1.00 | 12.96 | C |
| ATOM | 604 | C | LEU | A | 84 | 11.344 | 31.083 | 9.053 | 1.00 | 10.32 | C |
| ATOM | 605 | O | LEU | A | 84 | 10.454 | 30.792 | 8.237 | 1.00 | 9.88 | O |
| ATOM | 606 | N | ARG | A | 85 | 11.952 | 30.177 | 9.815 | 1.00 | 10.23 | N |
| ATOM | 607 | CA | ARG | A | 85 | 11.593 | 28.761 | 9.745 | 1.00 | 9.62 | C |
| ATOM | 608 | CB | ARG | A | 85 | 12.312 | 27.963 | 10.847 | 1.00 | 10.21 | C |
| ATOM | 609 | CG | ARG | A | 85 | 11.844 | 26.508 | 10.923 | 1.00 | 10.25 | C |
| ATOM | 610 | CD | ARG | A | 85 | 12.644 | 25.729 | 11.960 | 1.00 | 12.94 | C |
| ATOM | 611 | NE | ARG | A | 85 | 14.029 | 25.577 | 11.550 | 1.00 | 12.84 | N |
| ATOM | 612 | CZ | ARG | A | 85 | 14.949 | 24.921 | 12.249 | 1.00 | 16.33 | C |
| ATOM | 613 | NH1 | ARG | A | 85 | 14.632 | 24.365 | 13.420 | 1.00 | 18.08 | N |
| ATOM | 614 | NH2 | ARG | A | 85 | 16.184 | 24.836 | 11.765 | 1.00 | 16.17 | N |
| ATOM | 615 | C | ARG | A | 85 | 10.107 | 28.562 | 9.971 | 1.00 | 8.80 | C |
| ATOM | 616 | O | ARG | A | 85 | 9.591 | 29.077 | 10.949 | 1.00 | 10.26 | O |
| ATOM | 617 | N | GLU | A | 86 | 9.409 | 27.792 | 9.129 | 1.00 | 8.68 | N |
| ATOM | 618 | CA | GLU | A | 86 | 9.852 | 27.289 | 7.822 | 1.00 | 8.00 | C |
| ATOM | 619 | CB | GLU | A | 86 | 9.146 | 25.954 | 7.521 | 1.00 | 8.19 | C |
| ATOM | 620 | CG | GLU | A | 86 | 9.428 | 24.861 | 8.559 | 1.00 | 9.10 | C |
| ATOM | 621 | CD | GLU | A | 86 | 8.342 | 23.794 | 8.674 | 1.00 | 9.77 | C |
| ATOM | 622 | OE1 | GLU | A | 86 | 7.510 | 23.643 | 7.762 | 1.00 | 11.38 | O |
| ATOM | 623 | OE2 | GLU | A | 86 | 8.320 | 23.104 | 9.725 | 1.00 | 12.78 | O |
| ATOM | 624 | C | GLU | A | 86 | 9.437 | 28.274 | 6.742 | 1.00 | 7.90 | C |
| ATOM | 625 | O | GLU | A | 86 | 8.319 | 28.766 | 6.786 | 1.00 | 8.50 | O |
| ATOM | 626 | N | TYR | A | 87 | 10.292 | 28.539 | 5.754 | 1.00 | 7.01 | N |
| ATOM | 627 | CA | TYR | A | 87 | 9.869 | 29.427 | 4.672 | 1.00 | 7.34 | C |
| ATOM | 628 | CB | TYR | A | 87 | 11.011 | 30.335 | 4.156 | 1.00 | 7.65 | C |
| ATOM | 629 | CG | TYR | A | 87 | 10.515 | 31.364 | 3.174 | 1.00 | 7.73 | C |
| ATOM | 630 | CD1 | TYR | A | 87 | 10.061 | 32.602 | 3.624 | 1.00 | 8.89 | C |
| ATOM | 631 | CE1 | TYR | A | 87 | 9.543 | 33.548 | 2.727 | 1.00 | 9.48 | C |
| ATOM | 632 | CZ | TYR | A | 87 | 9.525 | 33.251 | 1.370 | 1.00 | 9.35 | C |
| ATOM | 633 | OH | TYR | A | 87 | 9.047 | 34.170 | 0.475 | 1.00 | 11.45 | O |
| ATOM | 634 | CE2 | TYR | A | 87 | 9.992 | 32.012 | 0.910 | 1.00 | 8.08 | C |
| ATOM | 635 | CD2 | TYR | A | 87 | 10.469 | 31.085 | 1.808 | 1.00 | 8.96 | C |
| ATOM | 636 | C | TYR | A | 87 | 9.237 | 28.585 | 3.556 | 1.00 | 6.85 | C |
| ATOM | 637 | O | TYR | A | 87 | 9.871 | 27.679 | 3.014 | 1.00 | 7.21 | O |
| ATOM | 638 | N | LEU | A | 88 | 7.989 | 28.873 | 3.222 | 1.00 | 7.27 | N |
| ATOM | 639 | CA | LEU | A | 88 | 7.232 | 28.062 | 2.268 | 1.00 | 7.42 | C |
| ATOM | 640 | CB | LEU | A | 88 | 5.731 | 28.206 | 2.569 | 1.00 | 7.51 | C |
| ATOM | 641 | CG | LEU | A | 88 | 4.811 | 27.408 | 1.644 | 1.00 | 6.76 | C |
| ATOM | 642 | CD1 | LEU | A | 88 | 5.041 | 25.889 | 1.840 | 1.00 | 8.93 | C |
| ATOM | 643 | CD2 | LEU | A | 88 | 3.353 | 27.778 | 1.883 | 1.00 | 8.72 | C |
| ATOM | 644 | C | LEU | A | 88 | 7.555 | 28.458 | 0.832 | 1.00 | 7.42 | C |
| ATOM | 645 | O | LEU | A | 88 | 7.187 | 29.557 | 0.384 | 1.00 | 7.91 | O |
| ATOM | 646 | N | HIS | A | 89 | 8.248 | 27.566 | 0.117 | 1.00 | 6.48 | N |
| ATOM | 647 | CA | HIS | A | 89 | 8.633 | 27.804 | −1.276 | 1.00 | 7.32 | C |
| ATOM | 648 | CB | HIS | A | 89 | 9.973 | 27.151 | −1.594 | 1.00 | 7.14 | C |
| ATOM | 649 | CG | HIS | A | 89 | 11.147 | 27.951 | −1.117 | 1.00 | 7.90 | C |
| ATOM | 650 | ND1 | HIS | A | 89 | 11.797 | 28.857 | −1.926 | 1.00 | 9.32 | N |
| ATOM | 651 | CE1 | HIS | A | 89 | 12.759 | 29.446 | −1.231 | 1.00 | 9.87 | C |
| ATOM | 652 | NE2 | HIS | A | 89 | 12.744 | 28.968 | −0.003 | 1.00 | 9.07 | N |
| ATOM | 653 | CD2 | HIS | A | 89 | 11.732 | 28.038 | 0.102 | 1.00 | 9.94 | C |
| ATOM | 654 | C | HIS | A | 89 | 7.615 | 27.386 | −2.328 | 1.00 | 7.51 | C |
| ATOM | 655 | O | HIS | A | 89 | 7.616 | 27.948 | −3.421 | 1.00 | 9.17 | O |
| ATOM | 656 | N | TRP | A | 90 | 6.740 | 26.429 | −2.007 | 1.00 | 8.06 | N |
| ATOM | 657 | CA | TRP | A | 90 | 5.845 | 25.845 | −3.012 | 1.00 | 7.76 | C |
| ATOM | 658 | CB | TRP | A | 90 | 6.687 | 24.975 | −3.954 | 1.00 | 7.32 | C |
| ATOM | 659 | CG | TRP | A | 90 | 6.069 | 24.527 | −5.241 | 1.00 | 8.01 | C |
| ATOM | 660 | CD1 | TRP | A | 90 | 5.055 | 25.112 | −5.967 | 1.00 | 8.69 | C |
| ATOM | 661 | NE1 | TRP | A | 90 | 4.816 | 24.372 | −7.113 | 1.00 | 9.57 | N |
| ATOM | 662 | CE2 | TRP | A | 90 | 5.704 | 23.322 | −7.155 | 1.00 | 8.74 | C |
| ATOM | 663 | CD2 | TRP | A | 90 | 6.507 | 23.396 | −6.000 | 1.00 | 7.96 | C |
| ATOM | 664 | CE3 | TRP | A | 90 | 7.505 | 22.419 | −5.804 | 1.00 | 7.84 | C |
| ATOM | 665 | CZ3 | TRP | A | 90 | 7.661 | 21.426 | −6.760 | 1.00 | 8.94 | C |
| ATOM | 666 | CH2 | TRP | A | 90 | 6.847 | 21.381 | −7.899 | 1.00 | 9.47 | C |
| ATOM | 667 | CZ2 | TRP | A | 90 | 5.864 | 22.311 | −8.113 | 1.00 | 8.43 | C |
| ATOM | 668 | C | TRP | A | 90 | 4.819 | 24.995 | −2.299 | 1.00 | 7.64 | C |
| ATOM | 669 | O | TRP | A | 90 | 5.151 | 24.325 | −1.319 | 1.00 | 7.71 | O |
| ATOM | 670 | N | LEU | A | 91 | 3.585 | 25.000 | −2.800 | 1.00 | 8.34 | N |
| ATOM | 671 | CA | LEU | A | 91 | 2.521 | 24.186 | −2.228 | 1.00 | 8.60 | C |
| ATOM | 672 | CB | LEU | A | 91 | 1.800 | 24.991 | −1.160 | 1.00 | 8.11 | C |
| ATOM | 673 | CG | LEU | A | 91 | 0.763 | 24.226 | −0.324 | 1.00 | 9.82 | C |
| ATOM | 674 | CD1 | LEU | A | 91 | 1.370 | 23.023 | 0.417 | 1.00 | 12.77 | C |
| ATOM | 675 | CD2 | LEU | A | 91 | 0.066 | 25.188 | 0.630 | 1.00 | 12.11 | C |
| ATOM | 676 | C | LEU | A | 91 | 1.562 | 23.779 | −3.342 | 1.00 | 9.49 | C |
| ATOM | 677 | O | LEU | A | 91 | 1.033 | 24.646 | −4.035 | 1.00 | 9.62 | O |
| ATOM | 678 | N | VAL | A | 92 | 1.359 | 22.470 | −3.501 | 1.00 | 9.41 | N |
| ATOM | 679 | CA | VAL | A | 92 | 0.487 | 21.941 | −4.538 | 1.00 | 10.23 | C |
| ATOM | 680 | CB | VAL | A | 92 | 1.298 | 21.302 | −5.677 | 1.00 | 9.84 | C |
| ATOM | 681 | CG1 | VAL | A | 92 | 0.403 | 20.846 | −6.831 | 1.00 | 10.65 | C |
| ATOM | 682 | CG2 | VAL | A | 92 | 2.385 | 22.268 | −6.209 | 1.00 | 10.59 | C |
| ATOM | 683 | C | VAL | A | 92 | −0.385 | 20.864 | −3.907 | 1.00 | 10.75 | C |
| ATOM | 684 | O | VAL | A | 92 | 0.100 | 20.023 | −3.132 | 1.00 | 11.18 | O |
| ATOM | 685 | N | THR | A | 93 | −1.667 | 20.857 | −4.251 | 1.00 | 10.91 | N |
| ATOM | 686 | CA | THR | A | 93 | −2.595 | 19.882 | −3.655 | 1.00 | 11.96 | C |
| ATOM | 687 | CB | THR | A | 93 | −3.603 | 20.559 | −2.727 | 1.00 | 12.12 | C |
| ATOM | 688 | OG1 | THR | A | 93 | −4.412 | 21.493 | −3.482 | 1.00 | 14.35 | O |
| ATOM | 689 | CG2 | THR | A | 93 | −2.862 | 21.282 | −1.592 | 1.00 | 14.25 | C |
| ATOM | 690 | C | THR | A | 93 | −3.338 | 19.117 | −4.742 | 1.00 | 11.26 | C |
| ATOM | 691 | O | THR | A | 93 | −3.227 | 19.450 | −5.904 | 1.00 | 11.58 | O |
| ATOM | 692 | N | ASP | A | 94 | −4.101 | 18.099 | −4.338 | 1.00 | 11.43 | N |
| ATOM | 693 | CA | ASP | A | 94 | −4.919 | 17.313 | −5.280 | 1.00 | 11.92 | C |
| ATOM | 694 | CB | ASP | A | 94 | −6.093 | 18.151 | −5.837 | 1.00 | 12.03 | C |
| ATOM | 695 | CG | ASP | A | 94 | −7.249 | 18.318 | −4.857 | 1.00 | 13.96 | C |
| ATOM | 696 | OD1 | ASP | A | 94 | −7.226 | 17.802 | −3.717 | 1.00 | 12.99 | O |
| ATOM | 697 | OD2 | ASP | A | 94 | −8.202 | 19.019 | −5.264 | 1.00 | 19.42 | O |
| ATOM | 698 | C | ASP | A | 94 | −4.078 | 16.789 | −6.442 | 1.00 | 11.54 | C |
| ATOM | 699 | O | ASP | A | 94 | −4.545 | 16.758 | −7.599 | 1.00 | 12.67 | O |
| ATOM | 700 | N | ILE | A | 95 | −2.847 | 16.350 | −6.158 | 1.00 | 11.13 | N |
| ATOM | 701 | CA | ILE | A | 95 | −2.035 | 15.704 | −7.184 | 1.00 | 10.88 | C |
| ATOM | 702 | CB | ILE | A | 95 | −0.543 | 15.699 | −6.806 | 1.00 | 10.33 | C |
| ATOM | 703 | CG1 | ILE | A | 95 | −0.030 | 17.136 | −6.564 | 1.00 | 10.03 | C |
| ATOM | 704 | CD1 | ILE | A | 95 | 1.299 | 17.205 | −5.814 | 1.00 | 10.35 | C |
| ATOM | 705 | CG2 | ILE | A | 95 | 0.257 | 14.960 | −7.877 | 1.00 | 11.90 | C |
| ATOM | 706 | C | ILE | A | 95 | −2.483 | 14.249 | −7.369 | 1.00 | 10.78 | C |
| ATOM | 707 | O | ILE | A | 95 | −2.428 | 13.475 | −6.415 | 1.00 | 11.23 | O |
| ATOM | 708 | N | PRO | A | 96 | −2.936 | 13.878 | −8.586 | 1.00 | 11.11 | N |
| ATOM | 709 | CA | PRO | A | 96 | −3.361 | 12.480 | −8.740 | 1.00 | 11.36 | C |
| ATOM | 710 | CB | PRO | A | 96 | −3.968 | 12.456 | −10.147 | 1.00 | 11.86 | C |
| ATOM | 711 | CG | PRO | A | 96 | −4.285 | 13.873 | −10.467 | 1.00 | 11.17 | C |
| ATOM | 712 | CD | PRO | A | 96 | −3.130 | 14.631 | −9.832 | 1.00 | 10.94 | C |
| ATOM | 713 | C | PRO | A | 96 | −2.208 | 11.495 | −8.667 | 1.00 | 11.30 | C |
| ATOM | 714 | O | PRO | A | 96 | −1.126 | 11.722 | −9.242 | 1.00 | 11.03 | O |
| ATOM | 715 | N | GLY | A | 97 | −2.456 | 10.375 | −7.996 | 1.00 | 11.13 | N |
| ATOM | 716 | CA | GLY | A | 97 | −1.457 | 9.323 | −7.898 | 1.00 | 12.00 | C |
| ATOM | 717 | C | GLY | A | 97 | −1.037 | 8.875 | −9.279 | 1.00 | 11.98 | C |
| ATOM | 718 | O | GLY | A | 97 | −1.829 | 8.921 | −10.232 | 1.00 | 11.74 | O |
| ATOM | 719 | N | THR | A | 98 | 0.220 | 8.452 | −9.374 | 1.00 | 13.09 | N |
| ATOM | 720 | CA | THR | A | 98 | 0.869 | 8.009 | −10.626 | 1.00 | 13.70 | C |
| ATOM | 721 | CB | THR | A | 98 | 0.148 | 6.814 | −11.366 | 1.00 | 13.74 | C |
| ATOM | 722 | OG1 | THR | A | 98 | −1.010 | 7.283 | −12.081 | 1.00 | 15.52 | O |
| ATOM | 723 | CG2 | THR | A | 98 | −0.243 | 5.728 | −10.394 | 1.00 | 14.60 | C |
| ATOM | 724 | C | THR | A | 98 | 1.139 | 9.149 | −11.604 | 1.00 | 14.27 | C |
| ATOM | 725 | O | THR | A | 98 | 1.608 | 8.908 | −12.708 | 1.00 | 15.60 | O |
| ATOM | 726 | N | THR | A | 99 | 0.832 | 10.389 | −11.217 | 1.00 | 13.18 | N |
| ATOM | 727 | CA | THR | A | 99 | 1.107 | 11.527 | −12.102 | 1.00 | 12.89 | C |
| ATOM | 728 | CB | THR | A | 99 | −0.176 | 12.335 | −12.512 | 1.00 | 13.67 | C |
| ATOM | 729 | OG1 | THR | A | 99 | −0.656 | 13.144 | −11.429 | 1.00 | 12.87 | O |
| ATOM | 730 | CG2 | THR | A | 99 | −1.284 | 11.407 | −13.040 | 1.00 | 14.02 | C |
| ATOM | 731 | C | THR | A | 99 | 2.249 | 12.425 | −11.582 | 1.00 | 12.99 | C |
| ATOM | 732 | O | THR | A | 99 | 3.327 | 11.931 | −11.280 | 1.00 | 13.12 | O |
| ATOM | 733 | N | ALA | A | 100 | 2.033 | 13.734 | −11.529 | 1.00 | 12.68 | N |
| ATOM | 734 | CA | ALA | A | 100 | 3.090 | 14.657 | −11.117 | 1.00 | 11.77 | C |
| ATOM | 735 | CB | ALA | A | 100 | 4.032 | 14.965 | −12.298 | 1.00 | 12.20 | C |
| ATOM | 736 | C | ALA | A | 100 | 2.481 | 15.936 | −10.575 | 1.00 | 11.63 | C |
| ATOM | 737 | O | ALA | A | 100 | 1.296 | 16.215 | −10.811 | 1.00 | 10.64 | O |
| ATOM | 738 | N | ALA | A | 101 | 3.294 | 16.743 | −9.889 | 1.00 | 11.30 | N |
| ATOM | 739 | CA | ALA | A | 101 | 2.793 | 17.975 | −9.291 | 1.00 | 11.18 | C |
| ATOM | 740 | CB | ALA | A | 101 | 3.906 | 18.698 | −8.518 | 1.00 | 10.54 | C |
| ATOM | 741 | C | ALA | A | 101 | 2.143 | 18.922 | −10.312 | 1.00 | 12.00 | C |
| ATOM | 742 | O | ALA | A | 101 | 1.199 | 19.644 | −9.981 | 1.00 | 12.35 | O |
| ATOM | 743 | N | SER | A | 102 | 2.627 | 18.884 | −11.557 | 1.00 | 13.14 | N |
| ATOM | 744 | CA | SER | A | 102 | 2.049 | 19.712 | −12.629 | 1.00 | 14.55 | C |
| ATOM | 745 | CB | SER | A | 102 | 2.923 | 19.651 | −13.887 | 1.00 | 15.27 | C |
| ATOM | 746 | OG | SER | A | 102 | 3.029 | 18.337 | −14.373 | 1.00 | 17.69 | O |
| ATOM | 747 | C | SER | A | 102 | 0.581 | 19.383 | −12.956 | 1.00 | 14.98 | C |
| ATOM | 748 | O | SER | A | 102 | −0.114 | 20.176 | −13.603 | 1.00 | 14.30 | O |
| ATOM | 749 | N | PHE | A | 103 | 0.106 | 18.229 | −12.488 | 1.00 | 14.28 | N |
| ATOM | 750 | CA | PHE | A | 103 | −1.297 | 17.822 | −12.675 | 1.00 | 13.69 | C |
| ATOM | 751 | CB | PHE | A | 103 | −1.379 | 16.327 | −13.009 | 1.00 | 14.54 | C |
| ATOM | 752 | CG | PHE | A | 103 | −0.736 | 15.968 | −14.313 | 1.00 | 15.30 | C |
| ATOM | 753 | CD1 | PHE | A | 103 | 0.587 | 15.568 | −14.366 | 1.00 | 17.34 | C |
| ATOM | 754 | CE1 | PHE | A | 103 | 1.177 | 15.218 | −15.581 | 1.00 | 19.81 | C |
| ATOM | 755 | CZ | PHE | A | 103 | 0.435 | 15.284 | −16.763 | 1.00 | 19.20 | C |
| ATOM | 756 | CE2 | PHE | A | 103 | −0.885 | 15.708 | −16.724 | 1.00 | 21.07 | C |
| ATOM | 757 | CD2 | PHE | A | 103 | −1.469 | 16.031 | −15.498 | 1.00 | 19.05 | C |
| ATOM | 758 | C | PHE | A | 103 | −2.197 | 18.144 | −11.473 | 1.00 | 13.77 | C |
| ATOM | 759 | O | PHE | A | 103 | −3.398 | 17.874 | −11.487 | 1.00 | 14.00 | O |
| ATOM | 760 | N | GLY | A | 104 | −1.603 | 18.716 | −10.428 | 1.00 | 12.89 | N |
| ATOM | 761 | CA | GLY | A | 104 | −2.345 | 19.118 | −9.239 | 1.00 | 13.71 | C |
| ATOM | 762 | C | GLY | A | 104 | −2.793 | 20.576 | −9.280 | 1.00 | 13.46 | C |
| ATOM | 763 | O | GLY | A | 104 | −2.705 | 21.253 | −10.309 | 1.00 | 14.84 | O |
| ATOM | 764 | N | GLN | A | 105 | −3.278 | 21.045 | −8.140 | 1.00 | 13.60 | N |
| ATOM | 765 | CA | GLN | A | 105 | −3.740 | 22.417 | −7.952 | 1.00 | 13.67 | C |
| ATOM | 766 | CB | GLN | A | 105 | −5.004 | 22.420 | −7.067 | 1.00 | 15.08 | C |
| ATOM | 767 | CG | GLN | A | 105 | −5.539 | 23.801 | −6.654 | 1.00 | 18.75 | C |
| ATOM | 768 | CD | GLN | A | 105 | −6.079 | 24.610 | −7.822 | 1.00 | 24.42 | C |
| ATOM | 769 | OE1 | GLN | A | 105 | −6.881 | 24.113 | −8.613 | 1.00 | 25.27 | O |
| ATOM | 770 | NE2 | GLN | A | 105 | −5.640 | 25.861 | −7.935 | 1.00 | 24.80 | N |
| ATOM | 771 | C | GLN | A | 105 | −2.636 | 23.226 | −7.285 | 1.00 | 13.40 | C |
| ATOM | 772 | O | GLN | A | 105 | −2.291 | 22.951 | −6.140 | 1.00 | 13.14 | O |
| ATOM | 773 | N | GLU | A | 106 | −2.086 | 24.208 | −7.994 | 1.00 | 13.06 | N |
| ATOM | 774 | CA | GLU | A | 106 | −1.093 | 25.088 | −7.403 | 1.00 | 12.71 | C |
| ATOM | 775 | CB | GLU | A | 106 | −0.409 | 25.936 | −8.474 | 1.00 | 12.64 | C |
| ATOM | 776 | CG | GLU | A | 106 | 0.675 | 26.837 | −7.918 | 1.00 | 14.18 | C |
| ATOM | 777 | CD | GLU | A | 106 | 1.356 | 27.621 | −9.005 | 1.00 | 14.34 | C |
| ATOM | 778 | OE1 | GLU | A | 106 | 2.180 | 27.034 | −9.731 | 1.00 | 15.78 | O |
| ATOM | 779 | OE2 | GLU | A | 106 | 1.051 | 28.831 | −9.145 | 1.00 | 17.06 | O |
| ATOM | 780 | C | GLU | A | 106 | −1.762 | 25.959 | −6.356 | 1.00 | 12.69 | C |
| ATOM | 781 | O | GLU | A | 106 | −2.740 | 26.650 | −6.642 | 1.00 | 13.76 | O |
| ATOM | 782 | N | VAL | A | 107 | −1.274 | 25.876 | −5.127 | 1.00 | 11.35 | N |
| ATOM | 783 | CA | VAL | A | 107 | −1.785 | 26.718 | −4.035 | 1.00 | 11.75 | C |
| ATOM | 784 | CB | VAL | A | 107 | −2.046 | 25.870 | −2.783 | 1.00 | 11.64 | C |
| ATOM | 785 | CG1 | VAL | A | 107 | −2.493 | 26.751 | −1.606 | 1.00 | 12.09 | C |
| ATOM | 786 | CG2 | VAL | A | 107 | −3.099 | 24.796 | −3.090 | 1.00 | 13.27 | C |
| ATOM | 787 | C | VAL | A | 107 | −0.845 | 27.901 | −3.772 | 1.00 | 11.57 | C |
| ATOM | 788 | O | VAL | A | 107 | −1.287 | 29.035 | −3.574 | 1.00 | 12.74 | O |
| ATOM | 789 | N | MET | A | 108 | 0.460 | 27.635 | −3.771 | 1.00 | 10.59 | N |
| ATOM | 790 | CA | MET | A | 108 | 1.477 | 28.679 | −3.647 | 1.00 | 10.72 | C |
| ATOM | 791 | CB | MET | A | 108 | 2.168 | 28.601 | −2.283 | 1.00 | 10.85 | C |
| ATOM | 792 | CG | MET | A | 108 | 1.242 | 28.882 | −1.122 | 1.00 | 11.87 | C |
| ATOM | 793 | SD | MET | A | 108 | 0.513 | 30.546 | −1.129 | 1.00 | 14.91 | S |
| ATOM | 794 | CE | MET | A | 108 | 1.796 | 31.455 | −0.277 | 1.00 | 15.90 | C |
| ATOM | 795 | C | MET | A | 108 | 2.492 | 28.419 | −4.740 | 1.00 | 10.62 | C |
| ATOM | 796 | O | MET | A | 108 | 3.038 | 27.321 | −4.816 | 1.00 | 10.42 | O |
| ATOM | 797 | N | SER | A | 109 | 2.737 | 29.400 | −5.598 | 1.00 | 10.17 | N |
| ATOM | 798 | CA | SER | A | 109 | 3.697 | 29.261 | −6.696 | 1.00 | 10.51 | C |
| ATOM | 799 | CB | SER | A | 109 | 3.684 | 30.517 | −7.567 | 1.00 | 10.85 | C |
| ATOM | 800 | OG | SER | A | 109 | 4.113 | 31.646 | −6.818 | 1.00 | 13.34 | O |
| ATOM | 801 | C | SER | A | 109 | 5.113 | 29.005 | −6.182 | 1.00 | 10.03 | C |
| ATOM | 802 | O | SER | A | 109 | 5.473 | 29.392 | −5.072 | 1.00 | 9.10 | O |
| ATOM | 803 | N | TYR | A | 110 | 5.930 | 28.358 | −7.000 | 1.00 | 9.66 | N |
| ATOM | 804 | CA | TYR | A | 110 | 7.311 | 28.074 | −6.668 | 1.00 | 9.56 | C |
| ATOM | 805 | CB | TYR | A | 110 | 7.888 | 27.131 | −7.717 | 1.00 | 9.06 | C |
| ATOM | 806 | CG | TYR | A | 110 | 9.304 | 26.656 | −7.488 | 1.00 | 8.90 | C |
| ATOM | 807 | CD1 | TYR | A | 110 | 10.387 | 27.334 | −8.050 | 1.00 | 9.27 | C |
| ATOM | 808 | CE1 | TYR | A | 110 | 11.692 | 26.870 | −7.863 | 1.00 | 8.98 | C |
| ATOM | 809 | CZ | TYR | A | 110 | 11.938 | 25.739 | −7.106 | 1.00 | 8.80 | C |
| ATOM | 810 | OH | TYR | A | 110 | 13.212 | 25.238 | −6.923 | 1.00 | 9.40 | O |
| ATOM | 811 | CE2 | TYR | A | 110 | 10.869 | 25.058 | −6.532 | 1.00 | 9.65 | C |
| ATOM | 812 | CD2 | TYR | A | 110 | 9.567 | 25.515 | −6.738 | 1.00 | 8.74 | C |
| ATOM | 813 | C | TYR | A | 110 | 8.144 | 29.345 | −6.624 | 1.00 | 10.02 | C |
| ATOM | 814 | O | TYR | A | 110 | 8.211 | 30.087 | −7.629 | 1.00 | 11.20 | O |
| ATOM | 815 | N | GLU | A | 111 | 8.790 | 29.562 | −5.482 | 1.00 | 10.05 | N |
| ATOM | 816 | CA | GLU | A | 111 | 9.757 | 30.652 | −5.323 | 1.00 | 10.15 | C |
| ATOM | 817 | CB | GLU | A | 111 | 9.556 | 31.362 | −3.979 | 1.00 | 10.04 | C |
| ATOM | 818 | CG | GLU | A | 111 | 10.546 | 32.507 | −3.720 | 1.00 | 12.39 | C |
| ATOM | 819 | CD | GLU | A | 111 | 10.300 | 33.234 | −2.411 | 1.00 | 12.58 | C |
| ATOM | 820 | OE1 | GLU | A | 111 | 9.129 | 33.313 | −1.961 | 1.00 | 14.03 | O |
| ATOM | 821 | OE2 | GLU | A | 111 | 11.302 | 33.717 | −1.850 | 1.00 | 14.81 | O |
| ATOM | 822 | C | GLU | A | 111 | 11.168 | 30.071 | −5.380 | 1.00 | 10.67 | C |
| ATOM | 823 | O | GLU | A | 111 | 11.472 | 29.109 | −4.676 | 1.00 | 10.99 | O |
| ATOM | 824 | N | SER | A | 112 | 12.051 | 30.664 | −6.199 | 1.00 | 11.47 | N |
| ATOM | 825 | CA | SER | A | 112 | 13.434 | 30.181 | −6.328 | 1.00 | 12.27 | C |
| ATOM | 826 | CB | SER | A | 112 | 14.216 | 31.078 | −7.293 | 1.00 | 12.98 | C |
| ATOM | 827 | OG | SER | A | 112 | 13.543 | 31.240 | −8.518 | 1.00 | 14.60 | O |
| ATOM | 828 | C | SER | A | 112 | 14.208 | 30.149 | −5.011 | 1.00 | 12.28 | C |
| ATOM | 829 | O | SER | A | 112 | 14.293 | 31.172 | −4.326 | 1.00 | 13.14 | O |
| ATOM | 830 | N | PRO | A | 113 | 14.807 | 29.005 | −4.640 | 1.00 | 12.08 | N |
| ATOM | 831 | CA | PRO | A | 113 | 15.747 | 29.046 | −3.515 | 1.00 | 12.12 | C |
| ATOM | 832 | CB | PRO | A | 113 | 16.194 | 27.587 | −3.369 | 1.00 | 12.00 | C |
| ATOM | 833 | CG | PRO | A | 113 | 15.056 | 26.791 | −3.967 | 1.00 | 11.86 | C |
| ATOM | 834 | CD | PRO | A | 113 | 14.597 | 27.623 | −5.121 | 1.00 | 11.81 | C |
| ATOM | 835 | C | PRO | A | 113 | 16.960 | 29.931 | −3.798 | 1.00 | 13.02 | C |
| ATOM | 836 | O | PRO | A | 113 | 17.624 | 29.776 | −4.835 | 1.00 | 12.77 | O |
| ATOM | 837 | N | ARG | A | 114 | 17.264 | 30.832 | −2.864 | 1.00 | 12.67 | N |
| ATOM | 838 | CA | ARG | A | 114 | 18.442 | 31.712 | −2.969 | 1.00 | 14.34 | C |
| ATOM | 839 | CB | ARG | A | 114 | 17.999 | 33.128 | −3.318 | 1.00 | 15.11 | C |
| ATOM | 840 | CG | ARG | A | 114 | 17.260 | 33.237 | −4.656 | 1.00 | 18.32 | C |
| ATOM | 841 | CD | ARG | A | 114 | 16.893 | 34.673 | −4.969 | 1.00 | 26.56 | C |
| ATOM | 842 | NE | ARG | A | 114 | 16.152 | 35.293 | −3.878 | 1.00 | 31.69 | N |
| ATOM | 843 | CZ | ARG | A | 114 | 14.829 | 35.258 | −3.744 | 1.00 | 33.27 | C |
| ATOM | 844 | NH1 | ARG | A | 114 | 14.072 | 34.625 | −4.634 | 1.00 | 35.42 | N |
| ATOM | 845 | NH2 | ARG | A | 114 | 14.262 | 35.855 | −2.710 | 1.00 | 34.90 | N |
| ATOM | 846 | C | ARG | A | 114 | 19.233 | 31.670 | −1.651 | 1.00 | 13.74 | C |
| ATOM | 847 | O | ARG | A | 114 | 19.269 | 32.650 | −0.904 | 1.00 | 14.22 | O |
| ATOM | 848 | N | PRO | A | 115 | 19.866 | 30.526 | −1.344 | 1.00 | 13.88 | N |
| ATOM | 849 | CA | PRO | A | 115 | 20.507 | 30.380 | −0.033 | 1.00 | 13.86 | C |
| ATOM | 850 | CB | PRO | A | 115 | 20.974 | 28.917 | −0.035 | 1.00 | 14.45 | C |
| ATOM | 851 | CG | PRO | A | 115 | 21.146 | 28.598 | −1.489 | 1.00 | 14.04 | C |
| ATOM | 852 | CD | PRO | A | 115 | 20.034 | 29.326 | −2.175 | 1.00 | 13.73 | C |
| ATOM | 853 | C | PRO | A | 115 | 21.718 | 31.300 | 0.126 | 1.00 | 14.67 | C |
| ATOM | 854 | O | PRO | A | 115 | 22.486 | 31.487 | −0.822 | 1.00 | 15.81 | O |
| ATOM | 855 | N | THR | A | 116 | 21.846 | 31.890 | 1.306 | 1.00 | 14.57 | N |
| ATOM | 856 | CA | THR | A | 116 | 23.075 | 32.568 | 1.727 | 1.00 | 15.62 | C |
| ATOM | 857 | CB | THR | A | 116 | 22.992 | 34.107 | 1.613 | 1.00 | 15.85 | C |
| ATOM | 858 | OG1 | THR | A | 116 | 22.159 | 34.617 | 2.659 | 1.00 | 16.51 | O |
| ATOM | 859 | CG2 | THR | A | 116 | 22.463 | 34.566 | 0.247 | 1.00 | 14.60 | C |
| ATOM | 860 | C | THR | A | 116 | 23.362 | 32.205 | 3.189 | 1.00 | 16.16 | C |
| ATOM | 861 | O | THR | A | 116 | 22.523 | 31.608 | 3.901 | 1.00 | 15.19 | O |
| ATOM | 862 | N | MET | A | 117 | 24.555 | 32.588 | 3.637 | 1.00 | 17.12 | N |
| ATOM | 863 | CA | MET | A | 117 | 24.962 | 32.436 | 5.027 | 1.00 | 17.95 | C |
| ATOM | 864 | CB | MET | A | 117 | 24.107 | 33.320 | 5.941 | 1.00 | 17.77 | C |
| ATOM | 865 | CG | MET | A | 117 | 24.240 | 34.793 | 5.628 | 1.00 | 18.46 | C |
| ATOM | 866 | SD | MET | A | 117 | 23.239 | 35.830 | 6.716 | 1.00 | 20.64 | S |
| ATOM | 867 | CE | MET | A | 117 | 21.598 | 35.458 | 6.079 | 1.00 | 19.92 | C |
| ATOM | 868 | C | MET | A | 117 | 24.915 | 30.997 | 5.495 | 1.00 | 17.54 | C |
| ATOM | 869 | O | MET | A | 117 | 24.443 | 30.709 | 6.583 | 1.00 | 18.91 | O |
| ATOM | 870 | N | GLY | A | 118 | 25.397 | 30.090 | 4.665 | 1.00 | 17.48 | N |
| ATOM | 871 | CA | GLY | A | 118 | 25.508 | 28.706 | 5.068 | 1.00 | 16.34 | C |
| ATOM | 872 | C | GLY | A | 118 | 24.693 | 27.821 | 4.159 | 1.00 | 15.94 | C |
| ATOM | 873 | O | GLY | A | 118 | 24.062 | 28.296 | 3.218 | 1.00 | 16.77 | O |
| ATOM | 874 | N | ILE | A | 119 | 24.740 | 26.532 | 4.449 | 1.00 | 16.04 | N |
| ATOM | 875 | CA | ILE | A | 119 | 24.049 | 25.524 | 3.656 | 1.00 | 16.09 | C |
| ATOM | 876 | CB | ILE | A | 119 | 24.773 | 24.160 | 3.784 | 1.00 | 16.48 | C |
| ATOM | 877 | CG1 | ILE | A | 119 | 26.229 | 24.310 | 3.289 | 1.00 | 17.46 | C |
| ATOM | 878 | CD1 | ILE | A | 119 | 27.123 | 23.119 | 3.562 | 1.00 | 17.99 | C |
| ATOM | 879 | CG2 | ILE | A | 119 | 23.980 | 23.050 | 3.067 | 1.00 | 17.41 | C |
| ATOM | 880 | C | ILE | A | 119 | 22.579 | 25.425 | 4.099 | 1.00 | 15.46 | C |
| ATOM | 881 | O | ILE | A | 119 | 22.293 | 25.240 | 5.281 | 1.00 | 15.83 | O |
| ATOM | 882 | N | HIS | A | 120 | 21.662 | 25.559 | 3.138 | 1.00 | 14.92 | N |
| ATOM | 883 | CA | HIS | A | 120 | 20.217 | 25.451 | 3.391 | 1.00 | 13.59 | C |
| ATOM | 884 | CB | HIS | A | 120 | 19.433 | 26.511 | 2.617 | 1.00 | 13.32 | C |
| ATOM | 885 | CG | HIS | A | 120 | 19.646 | 27.916 | 3.083 | 1.00 | 13.00 | C |
| ATOM | 886 | ND1 | HIS | A | 120 | 18.611 | 28.813 | 3.220 | 1.00 | 11.82 | N |
| ATOM | 887 | CE1 | HIS | A | 120 | 19.089 | 29.973 | 3.630 | 1.00 | 13.67 | C |
| ATOM | 888 | NE2 | HIS | A | 120 | 20.399 | 29.861 | 3.747 | 1.00 | 13.30 | N |
| ATOM | 889 | CD2 | HIS | A | 120 | 20.774 | 28.588 | 3.415 | 1.00 | 13.44 | C |
| ATOM | 890 | C | HIS | A | 120 | 19.701 | 24.103 | 2.905 | 1.00 | 13.48 | C |
| ATOM | 891 | O | HIS | A | 120 | 20.095 | 23.625 | 1.836 | 1.00 | 13.83 | O |
| ATOM | 892 | N | ARG | A | 121 | 18.813 | 23.505 | 3.679 | 1.00 | 12.73 | N |
| ATOM | 893 | CA | ARG | A | 121 | 18.045 | 22.340 | 3.250 | 1.00 | 11.72 | C |
| ATOM | 894 | CB | ARG | A | 121 | 17.688 | 21.472 | 4.449 | 1.00 | 11.82 | C |
| ATOM | 895 | CG | ARG | A | 121 | 18.920 | 20.867 | 5.147 | 1.00 | 11.29 | C |
| ATOM | 896 | CD | ARG | A | 121 | 18.594 | 20.404 | 6.524 | 1.00 | 13.52 | C |
| ATOM | 897 | NE | ARG | A | 121 | 18.213 | 21.523 | 7.403 | 1.00 | 11.60 | N |
| ATOM | 898 | CZ | ARG | A | 121 | 17.640 | 21.367 | 8.598 | 1.00 | 12.37 | C |
| ATOM | 899 | NH1 | ARG | A | 121 | 17.367 | 20.156 | 9.075 | 1.00 | 15.43 | N |
| ATOM | 900 | NH2 | ARG | A | 121 | 17.327 | 22.451 | 9.314 | 1.00 | 13.12 | N |
| ATOM | 901 | C | ARG | A | 121 | 16.773 | 22.811 | 2.556 | 1.00 | 12.10 | C |
| ATOM | 902 | O | ARG | A | 121 | 16.029 | 23.624 | 3.100 | 1.00 | 12.34 | O |
| ATOM | 903 | N | LEU | A | 122 | 16.544 | 22.321 | 1.349 | 1.00 | 11.10 | N |
| ATOM | 904 | CA | LEU | A | 122 | 15.266 | 22.541 | 0.657 | 1.00 | 10.59 | C |
| ATOM | 905 | CB | LEU | A | 122 | 15.491 | 23.018 | −0.773 | 1.00 | 11.01 | C |
| ATOM | 906 | CG | LEU | A | 122 | 15.933 | 24.482 | −0.935 | 1.00 | 9.29 | C |
| ATOM | 907 | CD1 | LEU | A | 122 | 14.977 | 25.433 | −0.176 | 1.00 | 11.83 | C |
| ATOM | 908 | CD2 | LEU | A | 122 | 17.411 | 24.736 | −0.525 | 1.00 | 12.76 | C |
| ATOM | 909 | C | LEU | A | 122 | 14.514 | 21.226 | 0.692 | 1.00 | 10.81 | C |
| ATOM | 910 | O | LEU | A | 122 | 14.963 | 20.224 | 0.124 | 1.00 | 10.75 | O |
| ATOM | 911 | N | VAL | A | 123 | 13.400 | 21.223 | 1.408 | 1.00 | 9.83 | N |
| ATOM | 912 | CA | VAL | A | 123 | 12.737 | 20.010 | 1.845 | 1.00 | 9.54 | C |
| ATOM | 913 | CB | VAL | A | 123 | 12.494 | 20.036 | 3.395 | 1.00 | 9.97 | C |
| ATOM | 914 | CG1 | VAL | A | 123 | 11.776 | 18.785 | 3.869 | 1.00 | 11.17 | C |
| ATOM | 915 | CG2 | VAL | A | 123 | 13.815 | 20.228 | 4.182 | 1.00 | 10.12 | C |
| ATOM | 916 | C | VAL | A | 123 | 11.400 | 19.859 | 1.131 | 1.00 | 9.63 | C |
| ATOM | 917 | O | VAL | A | 123 | 10.573 | 20.765 | 1.180 | 1.00 | 9.57 | O |
| ATOM | 918 | N | PHE | A | 124 | 11.197 | 18.705 | 0.492 | 1.00 | 9.73 | N |
| ATOM | 919 | CA | PHE | A | 124 | 9.918 | 18.347 | −0.145 | 1.00 | 9.69 | C |
| ATOM | 920 | CB | PHE | A | 124 | 10.141 | 17.764 | −1.536 | 1.00 | 9.63 | C |
| ATOM | 921 | CG | PHE | A | 124 | 10.847 | 18.685 | −2.462 | 1.00 | 7.46 | C |
| ATOM | 922 | CD1 | PHE | A | 124 | 12.242 | 18.839 | −2.416 | 1.00 | 8.40 | C |
| ATOM | 923 | CE1 | PHE | A | 124 | 12.862 | 19.728 | −3.292 | 1.00 | 10.48 | C |
| ATOM | 924 | CZ | PHE | A | 124 | 12.102 | 20.466 | −4.186 | 1.00 | 8.85 | C |
| ATOM | 925 | CE2 | PHE | A | 124 | 10.741 | 20.321 | −4.220 | 1.00 | 8.33 | C |
| ATOM | 926 | CD2 | PHE | A | 124 | 10.116 | 19.455 | −3.362 | 1.00 | 8.77 | C |
| ATOM | 927 | C | PHE | A | 124 | 9.236 | 17.312 | 0.704 | 1.00 | 10.45 | C |
| ATOM | 928 | O | PHE | A | 124 | 9.887 | 16.331 | 1.086 | 1.00 | 11.33 | O |
| ATOM | 929 | N | VAL | A | 125 | 7.956 | 17.509 | 1.007 | 1.00 | 9.83 | N |
| ATOM | 930 | CA | VAL | A | 125 | 7.191 | 16.596 | 1.863 | 1.00 | 10.12 | C |
| ATOM | 931 | CB | VAL | A | 125 | 6.828 | 17.207 | 3.242 | 1.00 | 10.33 | C |
| ATOM | 932 | CG1 | VAL | A | 125 | 6.264 | 16.123 | 4.181 | 1.00 | 10.69 | C |
| ATOM | 933 | CG2 | VAL | A | 125 | 8.033 | 17.912 | 3.891 | 1.00 | 11.21 | C |
| ATOM | 934 | C | VAL | A | 125 | 5.909 | 16.245 | 1.119 | 1.00 | 11.31 | C |
| ATOM | 935 | O | VAL | A | 125 | 5.200 | 17.138 | 0.629 | 1.00 | 10.47 | O |
| ATOM | 936 | N | LEU | A | 126 | 5.622 | 14.946 | 1.017 | 1.00 | 11.31 | N |
| ATOM | 937 | CA | LEU | A | 126 | 4.429 | 14.477 | 0.317 | 1.00 | 13.02 | C |
| ATOM | 938 | CB | LEU | A | 126 | 4.811 | 13.458 | −0.760 | 1.00 | 13.12 | C |
| ATOM | 939 | CG | LEU | A | 126 | 3.734 | 13.135 | −1.797 | 1.00 | 13.99 | C |
| ATOM | 940 | CD1 | LEU | A | 126 | 3.462 | 14.325 | −2.725 | 1.00 | 11.96 | C |
| ATOM | 941 | CD2 | LEU | A | 126 | 4.124 | 11.908 | −2.618 | 1.00 | 13.59 | C |
| ATOM | 942 | C | LEU | A | 126 | 3.475 | 13.863 | 1.315 | 1.00 | 12.82 | C |
| ATOM | 943 | O | LEU | A | 126 | 3.893 | 13.033 | 2.128 | 1.00 | 12.99 | O |
| ATOM | 944 | N | PHE | A | 127 | 2.220 | 14.289 | 1.274 | 1.00 | 12.69 | N |
| ATOM | 945 | CA | PHE | A | 127 | 1.153 | 13.702 | 2.089 | 1.00 | 13.21 | C |
| ATOM | 946 | CB | PHE | A | 127 | 0.478 | 14.770 | 2.943 | 1.00 | 13.79 | C |
| ATOM | 947 | CG | PHE | A | 127 | 1.395 | 15.421 | 3.934 | 1.00 | 14.67 | C |
| ATOM | 948 | CD1 | PHE | A | 127 | 1.573 | 14.863 | 5.197 | 1.00 | 16.53 | C |
| ATOM | 949 | CE1 | PHE | A | 127 | 2.420 | 15.452 | 6.132 | 1.00 | 15.30 | C |
| ATOM | 950 | CZ | PHE | A | 127 | 3.109 | 16.621 | 5.800 | 1.00 | 14.76 | C |
| ATOM | 951 | CE2 | PHE | A | 127 | 2.933 | 17.190 | 4.533 | 1.00 | 15.46 | C |
| ATOM | 952 | CD2 | PHE | A | 127 | 2.074 | 16.595 | 3.610 | 1.00 | 14.89 | C |
| ATOM | 953 | C | PHE | A | 127 | 0.115 | 13.079 | 1.191 | 1.00 | 13.58 | C |
| ATOM | 954 | O | PHE | A | 127 | −0.165 | 13.571 | 0.098 | 1.00 | 12.54 | O |
| ATOM | 955 | N | GLN | A | 128 | −0.475 | 11.998 | 1.677 | 1.00 | 14.07 | N |
| ATOM | 956 | CA | GLN | A | 128 | −1.628 | 11.401 | 1.039 | 1.00 | 15.00 | C |
| ATOM | 957 | CB | GLN | A | 128 | −1.593 | 9.892 | 1.263 | 1.00 | 14.40 | C |
| ATOM | 958 | CG | GLN | A | 128 | −2.647 | 9.119 | 0.498 | 1.00 | 17.88 | C |
| ATOM | 959 | CD | GLN | A | 128 | −2.471 | 7.620 | 0.655 | 1.00 | 19.02 | C |
| ATOM | 960 | OE1 | GLN | A | 128 | −1.358 | 7.099 | 0.556 | 1.00 | 23.21 | O |
| ATOM | 961 | NE2 | GLN | A | 128 | −3.568 | 6.917 | 0.896 | 1.00 | 22.83 | N |
| ATOM | 962 | C | GLN | A | 128 | −2.898 | 11.984 | 1.647 | 1.00 | 14.63 | C |
| ATOM | 963 | O | GLN | A | 128 | −3.084 | 11.961 | 2.868 | 1.00 | 16.63 | O |
| ATOM | 964 | N | GLN | A | 129 | −3.763 | 12.525 | 0.797 | 1.00 | 15.09 | N |
| ATOM | 965 | CA | GLN | A | 129 | −5.054 | 13.067 | 1.206 | 1.00 | 14.67 | C |
| ATOM | 966 | CB | GLN | A | 129 | −5.546 | 14.050 | 0.154 | 1.00 | 15.37 | C |
| ATOM | 967 | CG | GLN | A | 129 | −4.582 | 15.224 | −0.057 | 1.00 | 13.79 | C |
| ATOM | 968 | CD | GLN | A | 129 | −4.934 | 16.116 | −1.247 | 1.00 | 13.40 | C |
| ATOM | 969 | OE1 | GLN | A | 129 | −4.049 | 16.816 | −1.769 | 1.00 | 13.01 | O |
| ATOM | 970 | NE2 | GLN | A | 129 | −6.204 | 16.101 | −1.698 | 1.00 | 11.64 | N |
| ATOM | 971 | C | GLN | A | 129 | −6.083 | 11.944 | 1.370 | 1.00 | 16.55 | C |
| ATOM | 972 | O | GLN | A | 129 | −5.941 | 10.873 | 0.766 | 1.00 | 16.37 | O |
| ATOM | 973 | N | LEU | A | 130 | −7.109 | 12.189 | 2.183 | 1.00 | 18.50 | N |
| ATOM | 974 | CA | LEU | A | 130 | −8.207 | 11.207 | 2.287 | 1.00 | 19.97 | C |
| ATOM | 975 | CB | LEU | A | 130 | −9.097 | 11.463 | 3.517 | 1.00 | 19.90 | C |
| ATOM | 976 | CG | LEU | A | 130 | −8.622 | 10.883 | 4.858 | 1.00 | 22.65 | C |
| ATOM | 977 | CD1 | LEU | A | 130 | −9.673 | 11.088 | 5.947 | 1.00 | 23.26 | C |
| ATOM | 978 | CD2 | LEU | A | 130 | −8.245 | 9.400 | 4.777 | 1.00 | 24.56 | C |
| ATOM | 979 | C | LEU | A | 130 | −9.055 | 11.182 | 1.022 | 1.00 | 20.25 | C |
| ATOM | 980 | O | LEU | A | 130 | −9.735 | 10.196 | 0.749 | 1.00 | 20.92 | O |
| ATOM | 981 | N | GLY | A | 131 | −9.024 | 12.284 | 0.277 | 1.00 | 20.14 | N |
| ATOM | 982 | CA | GLY | A | 131 | −9.701 | 12.458 | −1.015 | 1.00 | 19.37 | C |
| ATOM | 983 | C | GLY | A | 131 | −9.372 | 13.880 | −1.483 | 1.00 | 19.58 | C |
| ATOM | 984 | O | GLY | A | 131 | −8.575 | 14.563 | −0.841 | 1.00 | 18.85 | O |
| ATOM | 985 | N | ARG | A | 132 | −9.980 | 14.334 | −2.580 | 1.00 | 19.45 | N |
| ATOM | 986 | CA | ARG | A | 132 | −9.835 | 15.728 | −3.012 | 1.00 | 20.28 | C |
| ATOM | 987 | CB | ARG | A | 132 | −10.724 | 16.049 | −4.214 | 1.00 | 20.91 | C |
| ATOM | 988 | CG | ARG | A | 132 | −10.174 | 15.577 | −5.543 | 1.00 | 23.26 | C |
| ATOM | 989 | CD | ARG | A | 132 | −10.734 | 16.402 | −6.688 | 1.00 | 27.64 | C |
| ATOM | 990 | NE | ARG | A | 132 | −10.051 | 16.107 | −7.946 | 1.00 | 30.86 | N |
| ATOM | 991 | CZ | ARG | A | 132 | −10.573 | 15.390 | −8.940 | 1.00 | 32.62 | C |
| ATOM | 992 | NH1 | ARG | A | 132 | −11.804 | 14.898 | −8.847 | 1.00 | 33.66 | N |
| ATOM | 993 | NH2 | ARG | A | 132 | −9.868 | 15.180 | −10.042 | 1.00 | 32.84 | N |
| ATOM | 994 | C | ARG | A | 132 | −10.222 | 16.625 | −1.857 | 1.00 | 19.94 | C |
| ATOM | 995 | O | ARG | A | 132 | −11.180 | 16.343 | −1.130 | 1.00 | 20.21 | O |
| ATOM | 996 | N | GLN | A | 133 | −9.456 | 17.686 | −1.657 | 1.00 | 19.12 | N |
| ATOM | 997 | CA | GLN | A | 133 | −9.789 | 18.599 | −0.589 | 1.00 | 19.38 | C |
| ATOM | 998 | CB | GLN | A | 133 | −9.241 | 18.113 | 0.757 | 1.00 | 20.24 | C |
| ATOM | 999 | CG | GLN | A | 133 | −7.767 | 18.277 | 0.967 | 1.00 | 21.09 | C |
| ATOM | 1000 | CD | GLN | A | 133 | −7.299 | 17.544 | 2.211 | 1.00 | 23.17 | C |
| ATOM | 1001 | OE1 | GLN | A | 133 | −6.610 | 16.531 | 2.113 | 1.00 | 24.79 | O |
| ATOM | 1002 | NE2 | GLN | A | 133 | −7.697 | 18.034 | 3.389 | 1.00 | 23.32 | N |
| ATOM | 1003 | C | GLN | A | 133 | −9.389 | 20.016 | −0.903 | 1.00 | 18.56 | C |
| ATOM | 1004 | O | GLN | A | 133 | −8.573 | 20.275 | −1.789 | 1.00 | 19.53 | O |
| ATOM | 1005 | N | THR | A | 134 | −10.002 | 20.930 | −0.161 | 1.00 | 18.00 | N |
| ATOM | 1006 | CA | THR | A | 134 | −9.748 | 22.348 | −0.315 | 1.00 | 17.42 | C |
| ATOM | 1007 | CB | THR | A | 134 | −11.033 | 23.181 | −0.105 | 1.00 | 17.19 | C |
| ATOM | 1008 | OG1 | THR | A | 134 | −12.032 | 22.768 | −1.048 | 1.00 | 17.69 | O |
| ATOM | 1009 | CG2 | THR | A | 134 | −10.748 | 24.690 | −0.273 | 1.00 | 16.85 | C |
| ATOM | 1010 | C | THR | A | 134 | −8.693 | 22.765 | 0.693 | 1.00 | 16.68 | C |
| ATOM | 1011 | O | THR | A | 134 | −8.889 | 22.695 | 1.903 | 1.00 | 17.87 | O |
| ATOM | 1012 | N | VAL | A | 135 | −7.573 | 23.213 | 0.162 | 1.00 | 15.65 | N |
| ATOM | 1013 | CA | VAL | A | 135 | −6.499 | 23.747 | 0.955 | 1.00 | 15.37 | C |
| ATOM | 1014 | CB | VAL | A | 135 | −5.186 | 22.988 | 0.688 | 1.00 | 15.83 | C |
| ATOM | 1015 | CG1 | VAL | A | 135 | −4.030 | 23.678 | 1.386 | 1.00 | 15.56 | C |
| ATOM | 1016 | CG2 | VAL | A | 135 | −5.320 | 21.544 | 1.140 | 1.00 | 16.93 | C |
| ATOM | 1017 | C | VAL | A | 135 | −6.368 | 25.226 | 0.587 | 1.00 | 15.08 | C |
| ATOM | 1018 | O | VAL | A | 135 | −6.070 | 25.572 | −0.560 | 1.00 | 16.30 | O |
| ATOM | 1019 | N | TYR | A | 136 | −6.605 | 26.094 | 1.571 | 1.00 | 14.42 | N |
| ATOM | 1020 | CA | TYR | A | 136 | −6.497 | 27.522 | 1.364 | 1.00 | 14.68 | C |
| ATOM | 1021 | CB | TYR | A | 136 | −7.226 | 28.290 | 2.477 | 1.00 | 14.50 | C |
| ATOM | 1022 | CG | TYR | A | 136 | −8.715 | 28.024 | 2.534 | 1.00 | 14.01 | C |
| ATOM | 1023 | CD1 | TYR | A | 136 | −9.245 | 26.984 | 3.303 | 1.00 | 12.67 | C |
| ATOM | 1024 | CE1 | TYR | A | 136 | −10.627 | 26.755 | 3.351 | 1.00 | 13.07 | C |
| ATOM | 1025 | CZ | TYR | A | 136 | −11.480 | 27.582 | 2.622 | 1.00 | 12.92 | C |
| ATOM | 1026 | OH | TYR | A | 136 | −12.844 | 27.371 | 2.655 | 1.00 | 14.02 | O |
| ATOM | 1027 | CE2 | TYR | A | 136 | −10.964 | 28.606 | 1.844 | 1.00 | 14.17 | C |
| ATOM | 1028 | CD2 | TYR | A | 136 | −9.596 | 28.823 | 1.808 | 1.00 | 14.69 | C |
| ATOM | 1029 | C | TYR | A | 136 | −5.039 | 27.935 | 1.327 | 1.00 | 14.65 | C |
| ATOM | 1030 | O | TYR | A | 136 | −4.216 | 27.443 | 2.106 | 1.00 | 15.76 | O |
| ATOM | 1031 | N | ALA | A | 137 | −4.724 | 28.845 | 0.418 | 1.00 | 13.18 | N |
| ATOM | 1032 | CA | ALA | A | 137 | −3.364 | 29.399 | 0.357 | 1.00 | 12.67 | C |
| ATOM | 1033 | CB | ALA | A | 137 | −3.242 | 30.381 | −0.785 | 1.00 | 12.89 | C |
| ATOM | 1034 | C | ALA | A | 137 | −3.011 | 30.093 | 1.678 | 1.00 | 12.48 | C |
| ATOM | 1035 | O | ALA | A | 137 | −3.762 | 30.948 | 2.148 | 1.00 | 12.38 | O |
| ATOM | 1036 | N | PRO | A | 138 | −1.873 | 29.734 | 2.294 | 1.00 | 12.25 | N |
| ATOM | 1037 | CA | PRO | A | 138 | −1.389 | 30.479 | 3.445 | 1.00 | 12.45 | C |
| ATOM | 1038 | CB | PRO | A | 138 | 0.023 | 29.905 | 3.642 | 1.00 | 12.65 | C |
| ATOM | 1039 | CG | PRO | A | 138 | −0.158 | 28.475 | 3.235 | 1.00 | 12.72 | C |
| ATOM | 1040 | CD | PRO | A | 138 | −0.994 | 28.582 | 1.993 | 1.00 | 12.44 | C |
| ATOM | 1041 | C | PRO | A | 138 | −1.331 | 31.964 | 3.132 | 1.00 | 12.13 | C |
| ATOM | 1042 | O | PRO | A | 138 | −1.088 | 32.335 | 1.981 | 1.00 | 13.03 | O |
| ATOM | 1043 | N | GLY | A | 139 | −1.599 | 32.796 | 4.136 | 1.00 | 12.36 | N |
| ATOM | 1044 | CA | GLY | A | 139 | −1.580 | 34.241 | 3.947 | 1.00 | 12.46 | C |
| ATOM | 1045 | C | GLY | A | 139 | −0.193 | 34.840 | 3.792 | 1.00 | 12.60 | C |
| ATOM | 1046 | O | GLY | A | 139 | −0.050 | 35.987 | 3.403 | 1.00 | 13.35 | O |
| ATOM | 1047 | N | TRP | A | 140 | 0.839 | 34.053 | 4.104 | 1.00 | 12.05 | N |
| ATOM | 1048 | CA | TRP | A | 140 | 2.243 | 34.482 | 4.004 | 1.00 | 11.38 | C |
| ATOM | 1049 | CB | TRP | A | 140 | 2.613 | 35.436 | 5.159 | 1.00 | 11.60 | C |
| ATOM | 1050 | CG | TRP | A | 140 | 2.293 | 34.856 | 6.509 | 1.00 | 12.07 | C |
| ATOM | 1051 | CD1 | TRP | A | 140 | 3.077 | 34.002 | 7.246 | 1.00 | 11.39 | C |
| ATOM | 1052 | NE1 | TRP | A | 140 | 2.441 | 33.650 | 8.402 | 1.00 | 12.72 | N |
| ATOM | 1053 | CE2 | TRP | A | 140 | 1.213 | 34.256 | 8.446 | 1.00 | 11.61 | C |
| ATOM | 1054 | CD2 | TRP | A | 140 | 1.077 | 35.015 | 7.255 | 1.00 | 10.56 | C |
| ATOM | 1055 | CE3 | TRP | A | 140 | −0.099 | 35.753 | 7.054 | 1.00 | 12.34 | C |
| ATOM | 1056 | CZ3 | TRP | A | 140 | −1.090 | 35.704 | 8.021 | 1.00 | 12.25 | C |
| ATOM | 1057 | CH2 | TRP | A | 140 | −0.932 | 34.929 | 9.196 | 1.00 | 12.88 | C |
| ATOM | 1058 | CZ2 | TRP | A | 140 | 0.214 | 34.207 | 9.429 | 1.00 | 12.38 | C |
| ATOM | 1059 | C | TRP | A | 140 | 3.102 | 33.222 | 4.023 | 1.00 | 10.15 | C |
| ATOM | 1060 | O | TRP | A | 140 | 2.653 | 32.160 | 4.460 | 1.00 | 10.45 | O |
| ATOM | 1061 | N | ARG | A | 141 | 4.327 | 33.347 | 3.542 | 1.00 | 9.66 | N |
| ATOM | 1062 | CA | ARG | A | 141 | 5.212 | 32.198 | 3.400 | 1.00 | 8.72 | C |
| ATOM | 1063 | CB | ARG | A | 141 | 6.017 | 32.335 | 2.110 | 1.00 | 8.68 | C |
| ATOM | 1064 | CG | ARG | A | 141 | 5.188 | 32.246 | 0.838 | 1.00 | 9.36 | C |
| ATOM | 1065 | CD | ARG | A | 141 | 6.057 | 32.535 | −0.355 | 1.00 | 11.08 | C |
| ATOM | 1066 | NE | ARG | A | 141 | 5.326 | 32.504 | −1.622 | 1.00 | 10.70 | N |
| ATOM | 1067 | CZ | ARG | A | 141 | 5.378 | 31.523 | −2.526 | 1.00 | 9.22 | C |
| ATOM | 1068 | NH1 | ARG | A | 141 | 6.131 | 30.443 | −2.325 | 1.00 | 9.69 | N |
| ATOM | 1069 | NH2 | ARG | A | 141 | 4.687 | 31.634 | −3.650 | 1.00 | 10.33 | N |
| ATOM | 1070 | C | ARG | A | 141 | 6.144 | 31.956 | 4.586 | 1.00 | 8.52 | C |
| ATOM | 1071 | O | ARG | A | 141 | 6.623 | 30.830 | 4.794 | 1.00 | 8.51 | O |
| ATOM | 1072 | N | GLN | A | 142 | 6.446 | 32.987 | 5.352 | 1.00 | 8.87 | N |
| ATOM | 1073 | CA | GLN | A | 142 | 7.421 | 32.823 | 6.422 | 1.00 | 9.56 | C |
| ATOM | 1074 | CB | GLN | A | 142 | 8.093 | 34.152 | 6.782 | 1.00 | 11.20 | C |
| ATOM | 1075 | CG | GLN | A | 142 | 7.277 | 35.053 | 7.669 | 1.00 | 13.08 | C |
| ATOM | 1076 | CD | GLN | A | 142 | 6.243 | 35.866 | 6.947 | 1.00 | 12.46 | C |
| ATOM | 1077 | OE1 | GLN | A | 142 | 6.078 | 35.794 | 5.716 | 1.00 | 13.85 | O |
| ATOM | 1078 | NE2 | GLN | A | 142 | 5.531 | 36.680 | 7.724 | 1.00 | 16.54 | N |
| ATOM | 1079 | C | GLN | A | 142 | 6.800 | 32.159 | 7.650 | 1.00 | 9.75 | C |
| ATOM | 1080 | O | GLN | A | 142 | 5.578 | 32.242 | 7.875 | 1.00 | 9.56 | O |
| ATOM | 1081 | N | ASN | A | 143 | 7.634 | 31.489 | 8.439 | 1.00 | 9.63 | N |
| ATOM | 1082 | CA | ASN | A | 143 | 7.174 | 30.875 | 9.689 | 1.00 | 9.86 | C |
| ATOM | 1083 | CB | ASN | A | 143 | 6.876 | 31.964 | 10.749 | 1.00 | 10.64 | C |
| ATOM | 1084 | CG | ASN | A | 143 | 8.100 | 32.796 | 11.086 | 1.00 | 11.63 | C |
| ATOM | 1085 | OD1 | ASN | A | 143 | 8.095 | 34.002 | 10.910 | 1.00 | 15.35 | O |
| ATOM | 1086 | ND2 | ASN | A | 143 | 9.169 | 32.143 | 11.537 | 1.00 | 12.42 | N |
| ATOM | 1087 | C | ASN | A | 143 | 5.983 | 29.936 | 9.476 | 1.00 | 10.55 | C |
| ATOM | 1088 | O | ASN | A | 143 | 5.035 | 29.881 | 10.276 | 1.00 | 11.27 | O |
| ATOM | 1089 | N | PHE | A | 144 | 6.034 | 29.231 | 8.348 | 1.00 | 9.68 | N |
| ATOM | 1090 | CA | PHE | A | 144 | 5.137 | 28.103 | 8.076 | 1.00 | 8.73 | C |
| ATOM | 1091 | CB | PHE | A | 144 | 5.272 | 27.713 | 6.595 | 1.00 | 9.14 | C |
| ATOM | 1092 | CG | PHE | A | 144 | 4.309 | 26.648 | 6.114 | 1.00 | 8.97 | C |
| ATOM | 1093 | CD1 | PHE | A | 144 | 3.004 | 26.973 | 5.770 | 1.00 | 8.19 | C |
| ATOM | 1094 | CE1 | PHE | A | 144 | 2.126 | 25.974 | 5.296 | 1.00 | 9.14 | C |
| ATOM | 1095 | CZ | PHE | A | 144 | 2.587 | 24.678 | 5.139 | 1.00 | 9.36 | C |
| ATOM | 1096 | CE2 | PHE | A | 144 | 3.900 | 24.359 | 5.472 | 1.00 | 8.56 | C |
| ATOM | 1097 | CD2 | PHE | A | 144 | 4.737 | 25.330 | 5.964 | 1.00 | 9.09 | C |
| ATOM | 1098 | C | PHE | A | 144 | 5.506 | 26.932 | 8.994 | 1.00 | 9.05 | C |
| ATOM | 1099 | O | PHE | A | 144 | 6.645 | 26.823 | 9.474 | 1.00 | 9.38 | O |
| ATOM | 1100 | N | ASN | A | 145 | 4.535 | 26.059 | 9.247 | 1.00 | 9.65 | N |
| ATOM | 1101 | CA | ASN | A | 145 | 4.789 | 24.883 | 10.064 | 1.00 | 10.23 | C |
| ATOM | 1102 | CB | ASN | A | 145 | 4.346 | 25.096 | 11.515 | 1.00 | 10.90 | C |
| ATOM | 1103 | CG | ASN | A | 145 | 4.847 | 24.012 | 12.409 | 1.00 | 12.57 | C |
| ATOM | 1104 | OD1 | ASN | A | 145 | 4.290 | 22.915 | 12.440 | 1.00 | 12.21 | O |
| ATOM | 1105 | ND2 | ASN | A | 145 | 5.936 | 24.284 | 13.115 | 1.00 | 14.68 | N |
| ATOM | 1106 | C | ASN | A | 145 | 4.102 | 23.685 | 9.459 | 1.00 | 10.44 | C |
| ATOM | 1107 | O | ASN | A | 145 | 2.880 | 23.566 | 9.516 | 1.00 | 10.47 | O |
| ATOM | 1108 | N | THR | A | 146 | 4.905 | 22.797 | 8.881 | 1.00 | 9.97 | N |
| ATOM | 1109 | CA | THR | A | 146 | 4.387 | 21.642 | 8.148 | 1.00 | 10.67 | C |
| ATOM | 1110 | CB | THR | A | 146 | 5.530 | 20.871 | 7.481 | 1.00 | 10.45 | C |
| ATOM | 1111 | OG1 | THR | A | 146 | 6.124 | 21.716 | 6.493 | 1.00 | 10.30 | O |
| ATOM | 1112 | CG2 | THR | A | 146 | 5.028 | 19.626 | 6.761 | 1.00 | 10.92 | C |
| ATOM | 1113 | C | THR | A | 146 | 3.551 | 20.737 | 9.043 | 1.00 | 11.55 | C |
| ATOM | 1114 | O | THR | A | 146 | 2.496 | 20.249 | 8.611 | 1.00 | 12.15 | O |
| ATOM | 1115 | N | LYS | A | 147 | 3.993 | 20.560 | 10.288 | 1.00 | 12.41 | N |
| ATOM | 1116 | CA | LYS | A | 147 | 3.218 | 19.752 | 11.254 | 1.00 | 13.55 | C |
| ATOM | 1117 | CB | LYS | A | 147 | 3.987 | 19.575 | 12.550 | 1.00 | 14.23 | C |
| ATOM | 1118 | CG | LYS | A | 147 | 5.138 | 18.616 | 12.473 | 1.00 | 17.93 | C |
| ATOM | 1119 | CD | LYS | A | 147 | 5.862 | 18.590 | 13.810 | 1.00 | 21.51 | C |
| ATOM | 1120 | CE | LYS | A | 147 | 7.079 | 17.707 | 13.760 | 1.00 | 23.93 | C |
| ATOM | 1121 | NZ | LYS | A | 147 | 7.807 | 17.739 | 15.066 | 1.00 | 26.40 | N |
| ATOM | 1122 | C | LYS | A | 147 | 1.860 | 20.386 | 11.534 | 1.00 | 13.72 | C |
| ATOM | 1123 | O | LYS | A | 147 | 0.832 | 19.705 | 11.512 | 1.00 | 14.53 | O |
| ATOM | 1124 | N | ASP | A | 148 | 1.823 | 21.687 | 11.786 | 1.00 | 13.45 | N |
| ATOM | 1125 | CA | ASP | A | 148 | 0.547 | 22.369 | 12.030 | 1.00 | 13.94 | C |
| ATOM | 1126 | CB | ASP | A | 148 | 0.763 | 23.832 | 12.406 | 1.00 | 14.55 | C |
| ATOM | 1127 | CG | ASP | A | 148 | 1.469 | 24.000 | 13.748 | 1.00 | 15.94 | C |
| ATOM | 1128 | OD1 | ASP | A | 148 | 1.539 | 23.014 | 14.527 | 1.00 | 18.07 | O |
| ATOM | 1129 | OD2 | ASP | A | 148 | 1.964 | 25.129 | 13.982 | 1.00 | 19.43 | O |
| ATOM | 1130 | C | ASP | A | 148 | −0.356 | 22.298 | 10.800 | 1.00 | 14.52 | C |
| ATOM | 1131 | O | ASP | A | 148 | −1.569 | 22.124 | 10.920 | 1.00 | 15.55 | O |
| ATOM | 1132 | N | PHE | A | 149 | 0.239 | 22.455 | 9.615 | 1.00 | 13.72 | N |
| ATOM | 1133 | CA | PHE | A | 149 | −0.484 | 22.427 | 8.350 | 1.00 | 13.57 | C |
| ATOM | 1134 | CB | PHE | A | 149 | 0.499 | 22.751 | 7.217 | 1.00 | 12.47 | C |
| ATOM | 1135 | CG | PHE | A | 149 | −0.055 | 22.561 | 5.832 | 1.00 | 11.03 | C |
| ATOM | 1136 | CD1 | PHE | A | 149 | −0.871 | 23.519 | 5.278 | 1.00 | 11.99 | C |
| ATOM | 1137 | CE1 | PHE | A | 149 | −1.374 | 23.361 | 3.986 | 1.00 | 11.02 | C |
| ATOM | 1138 | CZ | PHE | A | 149 | −1.058 | 22.221 | 3.258 | 1.00 | 11.51 | C |
| ATOM | 1139 | CE2 | PHE | A | 149 | −0.244 | 21.266 | 3.802 | 1.00 | 12.90 | C |
| ATOM | 1140 | CD2 | PHE | A | 149 | 0.261 | 21.431 | 5.086 | 1.00 | 12.02 | C |
| ATOM | 1141 | C | PHE | A | 149 | −1.149 | 21.064 | 8.138 | 1.00 | 14.01 | C |
| ATOM | 1142 | O | PHE | A | 149 | −2.322 | 20.998 | 7.779 | 1.00 | 13.74 | O |
| ATOM | 1143 | N | ALA | A | 150 | −0.397 | 19.990 | 8.359 | 1.00 | 15.01 | N |
| ATOM | 1144 | CA | ALA | A | 150 | −0.919 | 18.629 | 8.173 | 1.00 | 15.77 | C |
| ATOM | 1145 | CB | ALA | A | 150 | 0.173 | 17.609 | 8.285 | 1.00 | 15.18 | C |
| ATOM | 1146 | C | ALA | A | 150 | −2.040 | 18.343 | 9.165 | 1.00 | 16.79 | C |
| ATOM | 1147 | O | ALA | A | 150 | −3.004 | 17.642 | 8.835 | 1.00 | 17.16 | O |
| ATOM | 1148 | N | GLU | A | 151 | −1.919 | 18.897 | 10.367 | 1.00 | 17.48 | N |
| ATOM | 1149 | CA | GLU | A | 151 | −2.934 | 18.778 | 11.400 | 1.00 | 18.12 | C |
| ATOM | 1150 | CB | GLU | A | 151 | −2.417 | 19.372 | 12.714 | 1.00 | 18.66 | C |
| ATOM | 1151 | CG | GLU | A | 151 | −3.394 | 19.300 | 13.882 | 1.00 | 19.71 | C |
| ATOM | 1152 | CD | GLU | A | 151 | −3.723 | 17.876 | 14.291 | 1.00 | 22.61 | C |
| ATOM | 1153 | OE1 | GLU | A | 151 | −2.793 | 17.056 | 14.425 | 1.00 | 25.66 | O |
| ATOM | 1154 | OE2 | GLU | A | 151 | −4.923 | 17.588 | 14.479 | 1.00 | 23.68 | O |
| ATOM | 1155 | C | GLU | A | 151 | −4.216 | 19.458 | 10.951 | 1.00 | 18.07 | C |
| ATOM | 1156 | O | GLU | A | 151 | −5.280 | 18.829 | 10.983 | 1.00 | 18.28 | O |
| ATOM | 1157 | N | LEU | A | 152 | −4.112 | 20.716 | 10.506 | 1.00 | 17.59 | N |
| ATOM | 1158 | CA | LEU | A | 152 | −5.238 | 21.508 | 10.008 | 1.00 | 17.94 | C |
| ATOM | 1159 | CB | LEU | A | 152 | −4.766 | 22.887 | 9.508 | 1.00 | 17.76 | C |
| ATOM | 1160 | CG | LEU | A | 152 | −5.807 | 23.977 | 9.192 | 1.00 | 18.37 | C |
| ATOM | 1161 | CD1 | LEU | A | 152 | −6.685 | 24.308 | 10.420 | 1.00 | 21.33 | C |
| ATOM | 1162 | CD2 | LEU | A | 152 | −5.146 | 25.237 | 8.667 | 1.00 | 18.72 | C |
| ATOM | 1163 | C | LEU | A | 152 | −6.014 | 20.761 | 8.922 | 1.00 | 17.06 | C |
| ATOM | 1164 | O | LEU | A | 152 | −7.251 | 20.691 | 8.972 | 1.00 | 18.52 | O |
| ATOM | 1165 | N | TYR | A | 153 | −5.294 | 20.149 | 7.985 | 1.00 | 16.96 | N |
| ATOM | 1166 | CA | TYR | A | 153 | −5.924 | 19.549 | 6.809 | 1.00 | 16.72 | C |
| ATOM | 1167 | CB | TYR | A | 153 | −5.210 | 20.034 | 5.548 | 1.00 | 16.28 | C |
| ATOM | 1168 | CG | TYR | A | 153 | −5.463 | 21.496 | 5.309 | 1.00 | 14.51 | C |
| ATOM | 1169 | CD1 | TYR | A | 153 | −6.722 | 21.942 | 4.906 | 1.00 | 13.96 | C |
| ATOM | 1170 | CE1 | TYR | A | 153 | −6.988 | 23.292 | 4.692 | 1.00 | 14.15 | C |
| ATOM | 1171 | CZ | TYR | A | 153 | −5.987 | 24.238 | 4.887 | 1.00 | 14.55 | C |
| ATOM | 1172 | OH | TYR | A | 153 | −6.263 | 25.579 | 4.657 | 1.00 | 15.55 | O |
| ATOM | 1173 | CE2 | TYR | A | 153 | −4.722 | 23.823 | 5.299 | 1.00 | 14.25 | C |
| ATOM | 1174 | CD2 | TYR | A | 153 | −4.463 | 22.451 | 5.509 | 1.00 | 13.74 | C |
| ATOM | 1175 | C | TYR | A | 153 | −6.054 | 18.019 | 6.836 | 1.00 | 17.52 | C |
| ATOM | 1176 | O | TYR | A | 153 | −6.398 | 17.402 | 5.823 | 1.00 | 17.05 | O |
| ATOM | 1177 | N | ASN | A | 154 | −5.793 | 17.420 | 8.001 | 1.00 | 18.63 | N |
| ATOM | 1178 | CA | ASN | A | 154 | −5.824 | 15.953 | 8.167 | 1.00 | 18.72 | C |
| ATOM | 1179 | CB | ASN | A | 154 | −7.270 | 15.436 | 8.278 | 1.00 | 19.28 | C |
| ATOM | 1180 | CG | ASN | A | 154 | −7.341 | 13.994 | 8.742 | 1.00 | 21.31 | C |
| ATOM | 1181 | OD1 | ASN | A | 154 | −6.435 | 13.496 | 9.412 | 1.00 | 21.09 | O |
| ATOM | 1182 | ND2 | ASN | A | 154 | −8.416 | 13.307 | 8.367 | 1.00 | 23.10 | N |
| ATOM | 1183 | C | ASN | A | 154 | −5.050 | 15.201 | 7.081 | 1.00 | 19.12 | C |
| ATOM | 1184 | O | ASN | A | 154 | −5.574 | 14.307 | 6.404 | 1.00 | 19.41 | O |
| ATOM | 1185 | N | LEU | A | 155 | −3.790 | 15.570 | 6.926 | 1.00 | 18.73 | N |
| ATOM | 1186 | CA | LEU | A | 155 | −2.933 | 14.949 | 5.939 | 1.00 | 19.64 | C |
| ATOM | 1187 | CB | LEU | A | 155 | −1.990 | 16.001 | 5.355 | 1.00 | 19.37 | C |
| ATOM | 1188 | CG | LEU | A | 155 | −2.720 | 17.114 | 4.598 | 1.00 | 19.63 | C |
| ATOM | 1189 | CD1 | LEU | A | 155 | −1.735 | 18.155 | 4.151 | 1.00 | 17.67 | C |
| ATOM | 1190 | CD2 | LEU | A | 155 | −3.470 | 16.550 | 3.398 | 1.00 | 19.42 | C |
| ATOM | 1191 | C | LEU | A | 155 | −2.155 | 13.777 | 6.517 | 1.00 | 19.88 | C |
| ATOM | 1192 | O | LEU | A | 155 | −1.414 | 13.111 | 5.791 | 1.00 | 20.91 | O |
| ATOM | 1193 | N | GLY | A | 156 | −2.325 | 13.541 | 7.820 | 1.00 | 20.53 | N |
| ATOM | 1194 | CA | GLY | A | 156 | −1.637 | 12.458 | 8.532 | 1.00 | 20.50 | C |
| ATOM | 1195 | C | GLY | A | 156 | −0.141 | 12.671 | 8.603 | 1.00 | 20.23 | C |
| ATOM | 1196 | O | GLY | A | 156 | 0.318 | 13.812 | 8.697 | 1.00 | 21.15 | O |
| ATOM | 1197 | N | SER | A | 157 | 0.623 | 11.581 | 8.560 | 1.00 | 19.83 | N |
| ATOM | 1198 | CA | SER | A | 157 | 2.083 | 11.649 | 8.507 | 1.00 | 19.53 | C |
| ATOM | 1199 | CB | SER | A | 157 | 2.710 | 10.510 | 9.325 | 1.00 | 19.37 | C |
| ATOM | 1200 | OG | SER | A | 157 | 2.170 | 9.252 | 8.950 | 1.00 | 21.33 | O |
| ATOM | 1201 | C | SER | A | 157 | 2.564 | 11.582 | 7.060 | 1.00 | 18.45 | C |
| ATOM | 1202 | O | SER | A | 157 | 1.866 | 11.030 | 6.196 | 1.00 | 18.67 | O |
| ATOM | 1203 | N | PRO | A | 158 | 3.752 | 12.143 | 6.770 | 1.00 | 17.29 | N |
| ATOM | 1204 | CA | PRO | A | 158 | 4.268 | 12.102 | 5.398 | 1.00 | 16.75 | C |
| ATOM | 1205 | CB | PRO | A | 158 | 5.699 | 12.610 | 5.546 | 1.00 | 16.44 | C |
| ATOM | 1206 | CG | PRO | A | 158 | 5.665 | 13.478 | 6.758 | 1.00 | 16.01 | C |
| ATOM | 1207 | CD | PRO | A | 158 | 4.647 | 12.885 | 7.680 | 1.00 | 17.00 | C |
| ATOM | 1208 | C | PRO | A | 158 | 4.289 | 10.686 | 4.811 | 1.00 | 16.87 | C |
| ATOM | 1209 | O | PRO | A | 158 | 4.533 | 9.703 | 5.537 | 1.00 | 17.66 | O |
| ATOM | 1210 | N | VAL | A | 159 | 4.007 | 10.574 | 3.521 | 1.00 | 16.32 | N |
| ATOM | 1211 | CA | VAL | A | 159 | 4.219 | 9.317 | 2.812 | 1.00 | 15.92 | C |
| ATOM | 1212 | CB | VAL | A | 159 | 3.097 | 9.019 | 1.808 | 1.00 | 16.62 | C |
| ATOM | 1213 | CG1 | VAL | A | 159 | 1.790 | 8.823 | 2.538 | 1.00 | 16.77 | C |
| ATOM | 1214 | CG2 | VAL | A | 159 | 2.996 | 10.117 | 0.717 | 1.00 | 14.85 | C |
| ATOM | 1215 | C | VAL | A | 159 | 5.589 | 9.266 | 2.144 | 1.00 | 15.38 | C |
| ATOM | 1216 | O | VAL | A | 159 | 6.068 | 8.197 | 1.762 | 1.00 | 15.63 | O |
| ATOM | 1217 | N | ALA | A | 160 | 6.229 | 10.435 | 2.016 | 1.00 | 13.87 | N |
| ATOM | 1218 | CA | ALA | A | 160 | 7.543 | 10.547 | 1.428 | 1.00 | 13.29 | C |
| ATOM | 1219 | CB | ALA | A | 160 | 7.479 | 10.356 | −0.079 | 1.00 | 14.31 | C |
| ATOM | 1220 | C | ALA | A | 160 | 8.129 | 11.913 | 1.746 | 1.00 | 13.11 | C |
| ATOM | 1221 | O | ALA | A | 160 | 7.396 | 12.873 | 1.971 | 1.00 | 13.88 | O |
| ATOM | 1222 | N | ALA | A | 161 | 9.447 | 11.982 | 1.737 | 1.00 | 13.56 | N |
| ATOM | 1223 | CA | ALA | A | 161 | 10.127 | 13.268 | 1.794 | 1.00 | 14.00 | C |
| ATOM | 1224 | CB | ALA | A | 161 | 10.120 | 13.854 | 3.224 | 1.00 | 13.91 | C |
| ATOM | 1225 | C | ALA | A | 161 | 11.538 | 13.152 | 1.266 | 1.00 | 14.79 | C |
| ATOM | 1226 | O | ALA | A | 161 | 12.145 | 12.078 | 1.330 | 1.00 | 15.53 | O |
| ATOM | 1227 | N | VAL | A | 162 | 12.072 | 14.247 | 0.749 | 1.00 | 14.96 | N |
| ATOM | 1228 | CA | VAL | A | 162 | 13.433 | 14.273 | 0.254 | 1.00 | 14.10 | C |
| ATOM | 1229 | CB | VAL | A | 162 | 13.533 | 13.713 | −1.190 | 1.00 | 14.46 | C |
| ATOM | 1230 | CG1 | VAL | A | 162 | 13.041 | 14.726 | −2.200 | 1.00 | 13.96 | C |
| ATOM | 1231 | CG2 | VAL | A | 162 | 14.947 | 13.206 | −1.535 | 1.00 | 14.31 | C |
| ATOM | 1232 | C | VAL | A | 162 | 13.916 | 15.699 | 0.392 | 1.00 | 14.11 | C |
| ATOM | 1233 | O | VAL | A | 162 | 13.114 | 16.638 | 0.417 | 1.00 | 12.90 | O |
| ATOM | 1234 | N | TYR | A | 163 | 15.211 | 15.886 | 0.541 | 1.00 | 14.12 | N |
| ATOM | 1235 | CA | TYR | A | 163 | 15.731 | 17.231 | 0.567 | 1.00 | 14.32 | C |
| ATOM | 1236 | CB | TYR | A | 163 | 15.934 | 17.727 | 2.009 | 1.00 | 14.45 | C |
| ATOM | 1237 | CG | TYR | A | 163 | 17.152 | 17.144 | 2.711 | 1.00 | 14.76 | C |
| ATOM | 1238 | CD1 | TYR | A | 163 | 18.326 | 17.869 | 2.817 | 1.00 | 15.77 | C |
| ATOM | 1239 | CE1 | TYR | A | 163 | 19.460 | 17.312 | 3.456 | 1.00 | 14.86 | C |
| ATOM | 1240 | CZ | TYR | A | 163 | 19.379 | 16.047 | 3.987 | 1.00 | 15.49 | C |
| ATOM | 1241 | OH | TYR | A | 163 | 20.477 | 15.477 | 4.620 | 1.00 | 17.60 | O |
| ATOM | 1242 | CE2 | TYR | A | 163 | 18.218 | 15.319 | 3.892 | 1.00 | 15.78 | C |
| ATOM | 1243 | CD2 | TYR | A | 163 | 17.113 | 15.865 | 3.259 | 1.00 | 15.86 | C |
| ATOM | 1244 | C | TYR | A | 163 | 17.028 | 17.310 | −0.192 | 1.00 | 14.67 | C |
| ATOM | 1245 | O | TYR | A | 163 | 17.756 | 16.317 | −0.287 | 1.00 | 15.31 | O |
| ATOM | 1246 | N | PHE | A | 164 | 17.319 | 18.481 | −0.720 | 1.00 | 14.09 | N |
| ATOM | 1247 | CA | PHE | A | 164 | 18.651 | 18.772 | −1.196 | 1.00 | 13.71 | C |
| ATOM | 1248 | CB | PHE | A | 164 | 18.675 | 19.063 | −2.703 | 1.00 | 13.88 | C |
| ATOM | 1249 | CG | PHE | A | 164 | 17.966 | 20.310 | −3.124 | 1.00 | 13.15 | C |
| ATOM | 1250 | CD1 | PHE | A | 164 | 16.637 | 20.267 | −3.521 | 1.00 | 12.93 | C |
| ATOM | 1251 | CE1 | PHE | A | 164 | 15.995 | 21.427 | −3.968 | 1.00 | 10.81 | C |
| ATOM | 1252 | CZ | PHE | A | 164 | 16.686 | 22.628 | −4.039 | 1.00 | 12.04 | C |
| ATOM | 1253 | CE2 | PHE | A | 164 | 18.029 | 22.674 | −3.634 | 1.00 | 12.05 | C |
| ATOM | 1254 | CD2 | PHE | A | 164 | 18.653 | 21.518 | −3.211 | 1.00 | 12.96 | C |
| ATOM | 1255 | C | PHE | A | 164 | 19.289 | 19.863 | −0.358 | 1.00 | 14.36 | C |
| ATOM | 1256 | O | PHE | A | 164 | 18.598 | 20.613 | 0.346 | 1.00 | 13.83 | O |
| ATOM | 1257 | N | ASN | A | 165 | 20.612 | 19.920 | −0.395 | 1.00 | 15.33 | N |
| ATOM | 1258 | CA | ASN | A | 165 | 21.339 | 21.024 | 0.193 | 1.00 | 16.05 | C |
| ATOM | 1259 | CB | ASN | A | 165 | 22.604 | 20.506 | 0.879 | 1.00 | 16.27 | C |
| ATOM | 1260 | CG | ASN | A | 165 | 22.324 | 19.734 | 2.154 | 1.00 | 16.91 | C |
| ATOM | 1261 | OD1 | ASN | A | 165 | 22.900 | 18.653 | 2.381 | 1.00 | 20.15 | O |
| ATOM | 1262 | ND2 | ASN | A | 165 | 21.451 | 20.267 | 3.006 | 1.00 | 16.17 | N |
| ATOM | 1263 | C | ASN | A | 165 | 21.719 | 22.024 | −0.887 | 1.00 | 16.67 | C |
| ATOM | 1264 | O | ASN | A | 165 | 22.028 | 21.642 | −2.027 | 1.00 | 16.92 | O |
| ATOM | 1265 | N | SER | A | 166 | 21.719 | 23.306 | −0.541 | 1.00 | 16.19 | N |
| ATOM | 1266 | CA | SER | A | 166 | 22.118 | 24.361 | −1.453 | 1.00 | 16.96 | C |
| ATOM | 1267 | CB | SER | A | 166 | 20.909 | 24.890 | −2.255 | 1.00 | 16.85 | C |
| ATOM | 1268 | OG | SER | A | 166 | 21.302 | 25.638 | −3.398 | 1.00 | 16.80 | O |
| ATOM | 1269 | C | SER | A | 166 | 22.779 | 25.489 | −0.665 | 1.00 | 17.69 | C |
| ATOM | 1270 | O | SER | A | 166 | 22.361 | 25.816 | 0.453 | 1.00 | 16.27 | O |
| ATOM | 1271 | N | GLN | A | 167 | 23.815 | 26.070 | −1.250 | 1.00 | 18.62 | N |
| ATOM | 1272 | CA | GLN | A | 167 | 24.455 | 27.255 | −0.688 | 1.00 | 20.82 | C |
| ATOM | 1273 | CB | GLN | A | 167 | 25.731 | 26.875 | 0.077 | 1.00 | 20.52 | C |
| ATOM | 1274 | CG | GLN | A | 167 | 26.788 | 26.159 | −0.749 | 1.00 | 21.67 | C |
| ATOM | 1275 | CD | GLN | A | 167 | 27.973 | 25.753 | 0.083 | 1.00 | 22.50 | C |
| ATOM | 1276 | OE1 | GLN | A | 167 | 28.563 | 26.576 | 0.776 | 1.00 | 26.39 | O |
| ATOM | 1277 | NE2 | GLN | A | 167 | 28.326 | 24.471 | 0.035 | 1.00 | 24.55 | N |
| ATOM | 1278 | C | GLN | A | 167 | 24.738 | 28.248 | −1.810 | 1.00 | 21.81 | C |
| ATOM | 1279 | O | GLN | A | 167 | 24.673 | 27.888 | −2.994 | 1.00 | 21.58 | O |
| ATOM | 1280 | N | ARG | A | 168 | 25.034 | 29.494 | −1.443 | 1.00 | 23.44 | N |
| ATOM | 1281 | CA | ARG | A | 168 | 25.360 | 30.530 | −2.412 | 1.00 | 25.87 | C |
| ATOM | 1282 | CB | ARG | A | 168 | 25.821 | 31.796 | −1.686 | 1.00 | 25.72 | C |
| ATOM | 1283 | CG | ARG | A | 168 | 25.798 | 33.042 | −2.555 | 1.00 | 28.37 | C |
| ATOM | 1284 | CD | ARG | A | 168 | 26.821 | 34.067 | −2.092 | 1.00 | 32.14 | C |
| ATOM | 1285 | NE | ARG | A | 168 | 26.464 | 34.700 | −0.828 | 1.00 | 34.57 | N |
| ATOM | 1286 | CZ | ARG | A | 168 | 25.763 | 35.827 | −0.722 | 1.00 | 35.59 | C |
| ATOM | 1287 | NH1 | ARG | A | 168 | 25.318 | 36.448 | −1.808 | 1.00 | 36.78 | N |
| ATOM | 1288 | NH2 | ARG | A | 168 | 25.492 | 36.322 | 0.476 | 1.00 | 36.00 | N |
| ATOM | 1289 | C | ARG | A | 168 | 26.462 | 30.062 | −3.369 | 1.00 | 27.24 | C |
| ATOM | 1290 | O | ARG | A | 168 | 27.385 | 29.359 | −2.959 | 1.00 | 27.83 | O |
| ATOM | 1291 | N | GLU | A | 169 | 26.349 | 30.452 | −4.633 | 1.00 | 29.12 | N |
| ATOM | 1292 | CA | GLU | A | 169 | 27.375 | 30.149 | −5.627 | 1.00 | 31.26 | C |
| ATOM | 1293 | CB | GLU | A | 169 | 26.796 | 30.192 | −7.042 | 1.00 | 31.49 | C |
| ATOM | 1294 | CG | GLU | A | 169 | 26.098 | 28.916 | −7.452 | 1.00 | 33.07 | C |
| ATOM | 1295 | CD | GLU | A | 169 | 25.369 | 29.052 | −8.774 | 1.00 | 34.47 | C |
| ATOM | 1296 | OE1 | GLU | A | 169 | 26.041 | 29.085 | −9.832 | 1.00 | 36.26 | O |
| ATOM | 1297 | OE2 | GLU | A | 169 | 24.121 | 29.115 | −8.755 | 1.00 | 31.69 | O |
| ATOM | 1298 | C | GLU | A | 169 | 28.532 | 31.125 | −5.501 | 1.00 | 31.98 | C |
| ATOM | 1299 | O | GLU | A | 169 | 29.645 | 30.730 | −5.147 | 1.00 | 33.31 | O |
| ATOM | 1300 | N | ALA | A | 170 | 28.250 | 32.398 | −5.766 | 1.00 | 33.08 | N |
| ATOM | 1301 | CA | ALA | A | 170 | 29.251 | 33.461 | −5.678 | 1.00 | 34.02 | C |
| ATOM | 1302 | CB | ALA | A | 170 | 28.911 | 34.603 | −6.654 | 1.00 | 34.26 | C |
| ATOM | 1303 | C | ALA | A | 170 | 29.391 | 33.974 | −4.242 | 1.00 | 34.33 | C |
| ATOM | 1304 | O | ALA | A | 170 | 29.372 | 35.182 | −3.981 | 1.00 | 35.14 | O |
| ATOM | 1305 | O | HOH | C | 1 | −5.319 | 32.872 | 0.892 | 1.00 | 11.89 | O |
| ATOM | 1306 | O | HOH | C | 2 | 19.656 | 34.008 | 3.192 | 1.00 | 16.18 | O |
| ATOM | 1307 | O | HOH | C | 3 | −0.775 | 34.312 | 0.142 | 1.00 | 18.33 | O |
| ATOM | 1308 | O | HOH | C | 4 | 13.932 | 32.961 | −2.215 | 1.00 | 18.05 | O |
| ATOM | 1309 | O | HOH | C | 5 | 5.804 | 35.827 | 10.784 | 1.00 | 23.16 | O |
| ATOM | 1310 | O | HOH | C | 6 | 3.485 | 30.700 | 6.667 | 1.00 | 10.31 | O |
| ATOM | 1311 | O | HOH | C | 7 | 6.458 | 20.754 | −11.543 | 1.00 | 13.49 | O |
| ATOM | 1312 | O | HOH | C | 8 | 4.462 | 1.175 | 0.495 | 1.00 | 39.86 | O |
| ATOM | 1313 | O | HOH | C | 9 | 6.820 | 21.202 | 10.936 | 1.00 | 14.11 | O |
| ATOM | 1314 | O | HOH | C | 10 | 1.813 | 27.035 | 9.251 | 1.00 | 15.37 | O |
| ATOM | 1315 | O | HOH | C | 11 | 5.640 | 18.117 | −11.676 | 1.00 | 12.45 | O |
| ATOM | 1316 | O | HOH | C | 12 | 9.882 | 11.451 | 15.590 | 1.00 | 16.54 | O |
| ATOM | 1317 | O | HOH | C | 13 | 12.428 | 12.848 | 16.110 | 1.00 | 20.36 | O |
| ATOM | 1318 | O | HOH | C | 14 | −10.592 | 22.653 | 3.869 | 1.00 | 22.72 | O |
| ATOM | 1319 | O | HOH | C | 15 | 6.929 | 21.179 | −14.056 | 1.00 | 20.59 | O |
| ATOM | 1320 | O | HOH | C | 16 | −12.267 | 20.234 | −2.432 | 1.00 | 29.90 | O |
| ATOM | 1321 | O | HOH | C | 17 | 1.042 | 31.761 | −5.540 | 1.00 | 19.32 | O |
| ATOM | 1322 | O | HOH | C | 18 | −6.982 | 10.623 | −1.828 | 1.00 | 16.84 | O |
| ATOM | 1323 | O | HOH | C | 19 | 26.045 | 16.162 | −3.164 | 1.00 | 27.74 | O |
| ATOM | 1324 | O | HOH | C | 20 | 9.550 | 35.948 | 11.972 | 1.00 | 21.37 | O |
| ATOM | 1325 | O | HOH | C | 21 | 22.234 | 23.088 | 6.819 | 1.00 | 23.13 | O |
| ATOM | 1326 | O | HOH | C | 22 | −6.694 | 29.679 | −1.523 | 1.00 | 16.30 | O |
| ATOM | 1327 | O | HOH | C | 23 | −11.710 | 19.332 | 2.424 | 1.00 | 24.16 | O |
| ATOM | 1328 | O | HOH | C | 24 | −14.256 | 29.319 | 1.426 | 1.00 | 12.04 | O |
| ATOM | 1329 | O | HOH | C | 25 | 3.237 | 23.322 | −13.274 | 1.00 | 29.42 | O |
| ATOM | 1330 | O | HOH | C | 26 | 20.289 | 18.807 | −15.841 | 1.00 | 19.78 | O |
| ATOM | 1331 | O | HOH | C | 27 | −1.594 | 31.984 | 6.807 | 1.00 | 24.42 | O |
| ATOM | 1332 | O | HOH | C | 28 | 3.075 | 24.510 | −9.441 | 1.00 | 13.80 | O |
| ATOM | 1333 | O | HOH | C | 29 | 0.606 | 22.808 | −14.625 | 1.00 | 26.68 | O |
| ATOM | 1334 | O | HOH | C | 30 | 26.106 | 25.904 | 6.889 | 1.00 | 24.57 | O |
| ATOM | 1335 | O | HOH | C | 31 | −4.094 | 27.131 | 4.823 | 1.00 | 19.04 | O |
| ATOM | 1336 | O | HOH | C | 32 | 25.045 | 24.511 | −3.504 | 1.00 | 21.00 | O |
| ATOM | 1337 | O | HOH | C | 33 | 17.606 | 22.700 | −13.727 | 1.00 | 23.61 | O |
| ATOM | 1338 | O | HOH | C | 34 | 5.486 | 17.490 | −14.358 | 1.00 | 19.98 | O |
| ATOM | 1339 | O | HOH | C | 35 | 4.890 | 27.658 | −9.655 | 1.00 | 13.93 | O |
| ATOM | 1340 | O | HOH | C | 36 | 10.766 | 13.724 | −11.495 | 1.00 | 18.42 | O |
| ATOM | 1341 | O | HOH | C | 37 | 4.052 | 28.784 | 12.539 | 1.00 | 22.13 | O |
| ATOM | 1342 | O | HOH | C | 38 | 1.576 | 29.602 | 8.266 | 1.00 | 18.99 | O |
| ATOM | 1343 | O | HOH | C | 39 | 10.038 | 23.514 | 11.625 | 1.00 | 19.35 | O |
| ATOM | 1344 | O | HOH | C | 40 | 1.589 | 27.268 | 12.084 | 1.00 | 25.47 | O |
| ATOM | 1345 | O | HOH | C | 41 | 18.764 | 25.989 | 10.989 | 1.00 | 22.75 | O |
| ATOM | 1346 | O | HOH | C | 42 | 3.139 | 31.954 | 10.647 | 1.00 | 19.30 | O |
| ATOM | 1347 | O | HOH | C | 43 | −0.698 | 23.830 | −11.690 | 1.00 | 27.71 | O |
| ATOM | 1348 | O | HOH | C | 44 | 7.324 | 34.542 | −3.438 | 1.00 | 19.70 | O |
| ATOM | 1349 | O | HOH | C | 45 | 15.393 | 25.076 | −12.431 | 1.00 | 18.83 | O |
| ATOM | 1350 | O | HOH | C | 46 | −2.952 | 24.923 | −10.653 | 1.00 | 20.01 | O |
| ATOM | 1351 | O | HOH | C | 47 | 8.526 | 27.347 | −11.572 | 1.00 | 14.42 | O |
| ATOM | 1352 | O | HOH | C | 48 | 25.003 | 29.995 | 1.360 | 1.00 | 18.25 | O |
| ATOM | 1353 | O | HOH | C | 49 | 8.851 | 4.740 | −0.804 | 1.00 | 44.60 | O |
| ATOM | 1354 | O | HOH | C | 50 | 2.150 | 15.238 | 10.244 | 1.00 | 23.34 | O |
| ATOM | 1355 | O | HOH | C | 51 | 23.248 | 31.543 | −8.292 | 1.00 | 38.43 | O |
| ATOM | 1356 | O | HOH | C | 52 | 11.925 | 37.161 | 11.540 | 1.00 | 21.41 | O |
| ATOM | 1357 | O | HOH | C | 53 | 7.506 | 26.580 | 12.158 | 1.00 | 17.84 | O |
| ATOM | 1358 | O | HOH | C | 54 | 22.403 | 20.582 | 5.479 | 1.00 | 22.66 | O |
| ATOM | 1359 | O | HOH | C | 55 | 2.920 | 4.070 | −8.880 | 1.00 | 27.70 | O |
| ATOM | 1360 | O | HOH | C | 56 | 13.365 | 20.446 | −14.539 | 1.00 | 31.29 | O |
| ATOM | 1361 | O | HOH | C | 57 | 3.205 | 6.214 | 5.047 | 1.00 | 28.77 | O |
| ATOM | 1362 | O | HOH | C | 58 | 11.306 | 34.224 | 14.484 | 1.00 | 29.77 | O |
| ATOM | 1363 | O | HOH | C | 59 | 13.968 | 10.048 | 1.479 | 1.00 | 18.69 | O |
| ATOM | 1364 | O | HOH | C | 60 | 25.949 | 27.531 | 9.261 | 1.00 | 31.24 | O |
| ATOM | 1365 | O | HOH | C | 61 | 17.403 | 29.407 | −7.478 | 1.00 | 16.15 | O |
| ATOM | 1366 | O | HOH | C | 62 | 4.988 | 4.691 | −5.306 | 1.00 | 17.42 | O |
| ATOM | 1367 | O | HOH | C | 63 | 0.885 | 22.565 | −9.915 | 1.00 | 21.01 | O |
| ATOM | 1368 | O | HOH | C | 64 | 13.472 | 22.874 | −13.065 | 1.00 | 18.40 | O |
| ATOM | 1369 | O | HOH | C | 65 | 7.728 | 21.661 | 13.603 | 1.00 | 23.40 | O |
| ATOM | 1370 | O | HOH | C | 66 | 0.826 | 17.054 | 11.904 | 1.00 | 20.26 | O |
| ATOM | 1371 | O | HOH | C | 67 | 22.082 | 14.632 | −14.560 | 1.00 | 28.15 | O |
| ATOM | 1372 | O | HOH | C | 68 | −8.011 | 11.229 | −10.833 | 1.00 | 28.48 | O |
| ATOM | 1373 | O | HOH | C | 69 | 19.869 | 21.156 | −14.466 | 1.00 | 28.81 | O |
| ATOM | 1374 | O | HOH | C | 70 | 8.505 | 29.865 | −10.308 | 1.00 | 18.39 | O |
| ATOM | 1375 | O | HOH | C | 71 | 9.722 | 24.355 | −14.345 | 1.00 | 22.96 | O |
| ATOM | 1376 | O | HOH | C | 72 | −9.383 | 9.208 | −12.697 | 1.00 | 37.48 | O |
| ATOM | 1377 | O | HOH | C | 73 | 13.683 | 12.690 | −5.641 | 1.00 | 16.03 | O |
| ATOM | 1378 | O | HOH | C | 74 | 22.703 | 16.963 | 4.759 | 1.00 | 25.06 | O |
| ATOM | 1379 | O | HOH | C | 75 | −4.661 | 12.064 | 5.165 | 1.00 | 23.98 | O |
| ATOM | 1380 | O | HOH | C | 76 | −0.116 | 11.262 | 4.335 | 1.00 | 15.82 | O |
| ATOM | 1381 | O | HOH | C | 77 | 10.964 | 33.126 | −7.385 | 1.00 | 22.22 | O |
| ATOM | 1382 | O | HOH | C | 78 | 12.741 | 11.275 | −3.598 | 1.00 | 26.61 | O |
| ATOM | 1383 | O | HOH | C | 79 | 17.124 | 9.812 | −1.247 | 1.00 | 24.04 | O |
| ATOM | 1384 | O | HOH | C | 80 | 10.507 | 29.065 | 14.092 | 1.00 | 42.45 | O |
| ATOM | 1385 | O | HOH | C | 81 | 1.932 | 30.283 | −11.189 | 1.00 | 26.71 | O |
| ATOM | 1386 | O | HOH | C | 82 | 6.515 | 32.337 | −7.631 | 1.00 | 23.56 | O |
| ATOM | 1387 | O | HOH | C | 83 | 5.835 | 14.658 | 10.607 | 1.00 | 26.21 | O |
| ATOM | 1388 | O | HOH | C | 84 | 16.286 | 9.808 | −5.465 | 1.00 | 32.46 | O |
| ATOM | 1389 | O | HOH | C | 85 | 17.982 | 26.013 | −14.610 | 1.00 | 41.22 | O |
| ATOM | 1390 | O | HOH | C | 86 | 6.757 | 25.556 | −14.889 | 1.00 | 32.09 | O |
| ATOM | 1391 | O | HOH | C | 87 | 12.168 | 23.328 | 14.403 | 1.00 | 32.02 | O |
| ATOM | 1392 | O | HOH | C | 88 | 25.612 | 18.037 | 0.846 | 1.00 | 37.22 | O |
| ATOM | 1393 | O | HOH | C | 89 | −11.612 | 11.941 | −3.966 | 1.00 | 35.20 | O |
| ATOM | 1394 | O | HOH | C | 90 | 16.837 | 22.905 | 14.860 | 1.00 | 45.41 | O |
| ATOM | 1395 | O | HOH | C | 91 | 4.324 | 6.909 | 8.884 | 1.00 | 35.98 | O |
| ATOM | 1396 | O | HOH | C | 92 | −0.508 | 30.459 | −7.690 | 1.00 | 31.56 | O |
| ATOM | 1397 | O | HOH | C | 93 | 23.798 | 25.345 | −12.973 | 1.00 | 40.97 | O |
| ATOM | 1398 | O | HOH | C | 94 | −1.156 | 31.732 | −3.874 | 1.00 | 22.96 | O |
| ATOM | 1399 | O | HOH | C | 95 | 24.773 | 23.540 | 7.683 | 1.00 | 31.69 | O |
| ATOM | 1400 | O | HOH | C | 96 | 13.370 | 28.961 | 14.118 | 1.00 | 25.13 | O |
| ATOM | 1401 | O | HOH | C | 97 | 11.176 | 32.694 | −14.976 | 1.00 | 45.55 | O |
| ATOM | 1402 | O | HOH | C | 98 | 3.247 | 33.923 | −4.564 | 1.00 | 23.20 | O |
| ATOM | 1403 | O | HOH | C | 99 | 0.655 | 1.887 | −9.968 | 1.00 | 33.74 | O |
| ATOM | 1404 | O | HOH | C | 100 | −1.605 | 27.211 | 6.238 | 1.00 | 25.07 | O |
| ATOM | 1405 | O | HOH | C | 101 | 27.031 | 15.167 | −5.294 | 1.00 | 41.18 | O |
| ATOM | 1406 | O | HOH | C | 102 | 12.701 | 37.927 | −2.640 | 1.00 | 24.98 | O |
| ATOM | 1407 | O | HOH | C | 103 | 11.242 | 17.624 | 16.856 | 1.00 | 28.15 | O |
| ATOM | 1408 | O | HOH | C | 104 | −0.714 | 21.375 | 15.259 | 1.00 | 26.94 | O |
| ATOM | 1409 | O | HOH | C | 105 | 11.116 | 20.520 | −15.920 | 1.00 | 32.85 | O |
| ATOM | 1410 | O | HOH | C | 106 | 26.599 | 26.380 | −4.765 | 1.00 | 27.41 | O |
| ATOM | 1411 | O | HOH | C | 107 | −3.531 | 30.025 | −4.711 | 1.00 | 31.33 | O |
| ATOM | 1412 | O | HOH | C | 108 | −10.386 | 14.344 | 6.766 | 1.00 | 28.46 | O |
| ATOM | 1413 | O | HOH | C | 109 | −0.920 | 7.733 | −14.875 | 1.00 | 34.53 | O |
| ATOM | 1414 | O | HOH | C | 110 | 3.075 | 25.899 | −13.882 | 1.00 | 43.70 | O |
| ATOM | 1415 | O | HOH | C | 111 | 3.494 | 13.321 | 11.851 | 1.00 | 41.80 | O |
| ATOM | 1416 | O | HOH | C | 112 | 25.115 | 15.319 | −15.164 | 1.00 | 51.77 | O |
| ATOM | 1417 | O | HOH | C | 113 | −10.814 | 3.486 | −8.023 | 1.00 | 53.15 | O |
| ATOM | 1418 | O | HOH | C | 114 | −7.002 | 14.595 | 3.801 | 1.00 | 22.94 | O |
| ATOM | 1419 | O | HOH | C | 115 | 22.295 | 23.908 | 11.184 | 1.00 | 37.30 | O |
| ATOM | 1420 | O | HOH | C | 116 | 10.413 | 13.556 | −14.122 | 1.00 | 31.05 | O |
| ATOM | 1421 | O | HOH | C | 117 | 18.144 | 8.726 | 1.052 | 1.00 | 25.56 | O |
| ATOM | 1422 | O | HOH | C | 118 | 8.111 | 12.858 | 17.399 | 1.00 | 26.70 | O |
| ATOM | 1423 | O | HOH | C | 119 | −1.900 | 6.427 | −5.476 | 1.00 | 37.96 | O |
| ATOM | 1424 | O | HOH | C | 120 | 26.236 | 17.529 | −6.312 | 1.00 | 42.03 | O |
| ATOM | 1425 | O | HOH | C | 121 | 14.867 | 33.188 | −10.053 | 1.00 | 29.76 | O |
| ATOM | 1426 | O | HOH | C | 122 | 18.107 | 30.034 | −14.340 | 1.00 | 29.39 | O |
| ATOM | 1427 | O | HOH | C | 123 | 26.086 | 20.699 | 0.543 | 1.00 | 39.98 | O |
| ATOM | 1428 | O | HOH | C | 124 | 21.347 | 13.146 | 4.294 | 1.00 | 42.70 | O |
| ATOM | 1429 | O | HOH | C | 125 | −5.395 | 27.500 | −5.225 | 1.00 | 32.34 | O |
| ATOM | 1430 | O | HOH | C | 126 | 19.591 | 11.720 | −11.515 | 1.00 | 26.26 | O |
| ATOM | 1431 | O | HOH | C | 127 | −6.132 | 8.228 | 1.360 | 1.00 | 31.16 | O |
| ATOM | 1432 | O | HOH | C | 128 | 5.359 | 23.142 | −15.118 | 1.00 | 29.53 | O |
| ATOM | 1433 | O | HOH | C | 129 | 28.708 | 29.233 | −0.696 | 1.00 | 46.73 | O |
| ATOM | 1434 | O | HOH | C | 130 | 10.613 | 5.449 | 6.251 | 1.00 | 28.10 | O |
| ATOM | 1435 | O | HOH | C | 131 | −0.007 | 6.005 | −1.297 | 1.00 | 32.11 | O |
| ATOM | 1436 | O | HOH | C | 132 | 23.368 | 15.050 | 11.673 | 1.00 | 37.46 | O |
| ATOM | 1437 | O | HOH | C | 133 | 25.046 | 19.722 | −8.120 | 1.00 | 33.05 | O |
| ATOM | 1438 | O | HOH | C | 134 | 9.424 | 25.141 | 13.565 | 1.00 | 33.26 | O |
| ATOM | 1439 | O | HOH | C | 135 | 25.847 | 14.252 | 11.288 | 1.00 | 34.74 | O |
| ATOM | 1440 | O | HOH | C | 136 | 24.817 | 29.704 | 8.899 | 1.00 | 32.97 | O |
| ATOM | 1441 | O | HOH | C | 137 | 24.296 | 33.831 | 12.484 | 1.00 | 33.85 | O |
| ATOM | 1442 | O | HOH | C | 138 | 8.700 | 27.899 | −14.193 | 1.00 | 28.62 | O |
| ATOM | 1443 | O | HOH | C | 139 | 23.158 | 11.855 | −6.761 | 1.00 | 44.27 | O |
| ATOM | 1444 | O | HOH | C | 140 | 5.534 | 11.801 | 10.878 | 1.00 | 29.38 | O |
| ATOM | 1445 | O | HOH | C | 141 | −5.645 | 31.000 | −3.651 | 1.00 | 30.23 | O |
| ATOM | 1446 | O | HOH | C | 142 | 15.884 | 18.585 | 15.441 | 1.00 | 38.52 | O |
| ATOM | 1447 | O | HOH | C | 143 | 1.856 | 16.886 | 14.584 | 1.00 | 31.21 | O |
| ATOM | 1448 | O | HOH | C | 144 | 29.245 | 25.474 | −4.694 | 1.00 | 42.40 | O |
| ATOM | 1449 | O | HOH | C | 145 | −8.458 | 20.496 | −7.555 | 1.00 | 33.58 | O |
| ATOM | 1450 | O | HOH | C | 146 | −0.833 | 29.488 | 7.418 | 1.00 | 24.32 | O |
| ATOM | 1451 | O | HOH | C | 147 | 5.247 | 30.037 | −11.175 | 1.00 | 35.52 | O |
| ATOM | 1452 | O | HOH | C | 148 | 9.483 | 21.937 | −14.176 | 1.00 | 30.20 | O |
| ATOM | 1453 | O | HOH | C | 149 | 15.295 | 20.153 | 18.089 | 1.00 | 48.34 | O |
| ATOM | 1454 | O | HOH | C | 150 | 0.542 | 30.543 | 10.777 | 1.00 | 38.34 | O |
| ATOM | 1455 | O | HOH | C | 151 | 9.387 | 2.527 | 3.565 | 1.00 | 30.25 | O |
| ATOM | 1456 | O | HOH | C | 152 | 18.283 | 19.624 | 14.935 | 1.00 | 31.09 | O |
| ATOM | 1457 | O | HOH | C | 153 | 2.231 | 32.712 | −10.463 | 1.00 | 37.34 | O |
| ATOM | 1458 | O | HOH | C | 154 | 3.494 | 26.236 | 15.976 | 1.00 | 35.76 | O |
| ATOM | 1459 | O | HOH | C | 155 | 0.000 | 0.000 | −3.140 | 0.33 | 36.16 | O |
| ATOM | 1460 | O | HOH | C | 156 | 29.599 | 26.684 | 4.137 | 1.00 | 49.58 | O |
| ATOM | 1461 | O | HOH | C | 157 | 20.265 | 30.811 | −13.355 | 1.00 | 32.27 | O |
| ATOM | 1462 | O | HOH | C | 158 | 4.118 | 21.799 | 15.115 | 1.00 | 43.85 | O |
| ATOM | 1463 | O | HOH | C | 159 | −1.282 | 15.362 | 11.929 | 1.00 | 38.31 | O |
| ATOM | 1464 | O | HOH | C | 160 | 7.000 | 32.634 | 14.785 | 1.00 | 58.41 | O |
| ATOM | 1465 | O | HOH | C | 161 | −11.662 | 18.007 | −11.387 | 1.00 | 37.98 | O |
| ATOM | 1466 | O | HOH | C | 162 | 8.620 | 38.125 | 12.742 | 1.00 | 41.89 | O |
| ATOM | 1467 | O | HOH | C | 163 | 0.755 | 6.442 | 8.564 | 1.00 | 37.57 | O |
| ATOM | 1468 | O | HOH | C | 164 | 9.657 | 19.932 | 14.077 | 1.00 | 33.41 | O |
| ATOM | 1469 | O | HOH | C | 165 | 20.648 | 32.613 | −5.684 | 1.00 | 34.32 | O |
| ATOM | 1470 | O | HOH | C | 166 | −2.891 | 4.747 | −3.845 | 1.00 | 36.48 | O |
| ATOM | 1471 | O | HOH | C | 167 | 0.599 | 8.421 | 11.145 | 1.00 | 51.34 | O |
| ATOM | 1472 | O | HOH | C | 168 | 24.255 | 8.505 | 6.368 | 1.00 | 58.21 | O |
| ATOM | 1473 | O | HOH | C | 169 | 4.598 | 7.586 | 11.519 | 1.00 | 51.50 | O |
| ATOM | 1474 | O | HOH | C | 170 | 6.277 | 12.729 | −15.260 | 1.00 | 36.41 | O |
| ATOM | 1475 | O | HOH | C | 171 | 2.242 | 33.629 | −6.905 | 1.00 | 36.85 | O |
| ATOM | 1476 | O | HOH | C | 172 | −4.826 | 29.572 | 5.929 | 1.00 | 25.80 | O |
| ATOM | 1477 | O | HOH | C | 173 | 2.215 | 34.911 | 0.535 | 1.00 | 31.20 | O |
| ATOM | 1478 | O | HOH | C | 174 | 18.906 | 31.815 | −7.966 | 1.00 | 26.96 | O |
| ATOM | 1479 | O | HOH | C | 175 | 5.859 | 36.246 | −1.608 | 1.00 | 19.07 | O |
| ATOM | 1480 | O | HOH | C | 176 | 27.506 | 12.770 | 9.118 | 1.00 | 36.92 | O |
| ATOM | 1481 | O | HOH | C | 177 | −2.231 | 5.718 | 7.807 | 1.00 | 31.70 | O |
| ATOM | 1482 | O | HOH | C | 178 | −9.593 | 14.887 | 4.372 | 1.00 | 32.26 | O |
| ATOM | 1483 | O | HOH | C | 179 | 24.429 | 26.258 | 11.604 | 1.00 | 36.78 | O |
| ATOM | 1484 | O | HOH | C | 180 | 26.276 | 10.158 | 6.989 | 1.00 | 40.95 | O |
| ATOM | 1485 | O | HOH | C | 181 | 15.659 | 31.633 | −14.927 | 1.00 | 33.21 | O |
| ATOM | 1486 | O | HOH | C | 182 | 6.439 | 5.851 | 12.056 | 1.00 | 40.31 | O |
| ATOM | 1487 | O | HOH | C | 183 | 23.338 | 20.398 | 14.296 | 1.00 | 46.16 | O |
| ATOM | 1488 | O | HOH | C | 184 | −7.590 | 15.805 | −11.158 | 1.00 | 30.12 | O |
| ATOM | 1489 | O | HOH | C | 185 | 25.223 | 36.478 | −5.843 | 1.00 | 51.46 | O |
| ATOM | 1490 | O | HOH | C | 186 | 14.494 | 32.637 | −12.694 | 1.00 | 38.92 | O |
| ATOM | 1491 | O | HOH | C | 187 | −1.688 | 32.074 | 11.590 | 1.00 | 43.26 | O |
| ATOM | 1492 | O | HOH | C | 188 | 9.145 | 35.161 | −11.070 | 1.00 | 42.52 | O |
| ATOM | 1493 | O | HOH | C | 189 | 25.406 | 16.009 | 8.614 | 1.00 | 54.32 | O |
| ATOM | 1494 | O | HOH | C | 190 | 23.108 | 17.782 | 14.398 | 1.00 | 49.04 | O |
| ATOM | 1495 | O | HOH | C | 191 | 4.797 | 35.701 | 2.113 | 1.00 | 13.86 | O |
| ATOM | 1496 | O | HOH | C | 192 | 11.313 | 8.379 | 9.732 | 1.00 | 14.54 | O |
| ATOM | 1497 | O | HOH | C | 193 | 25.138 | 8.254 | 9.504 | 1.00 | 40.56 | O |
| ATOM | 1498 | O | HOH | C | 194 | −5.248 | 31.572 | 4.359 | 1.00 | 16.20 | O |
| ATOM | 1499 | O | HOH | C | 195 | 3.552 | 34.777 | −1.836 | 1.00 | 16.79 | O |
| ATOM | 1500 | O | HOH | C | 196 | 15.843 | 31.625 | −0.658 | 1.00 | 14.25 | O |
| ATOM | 1501 | O | HOH | C | 197 | 18.260 | 37.087 | 9.650 | 1.00 | 29.40 | O |
| ATOM | 1502 | O | HOH | C | 198 | 6.016 | 16.515 | 17.366 | 1.00 | 32.02 | O |
| ATOM | 1503 | O | HOH | C | 199 | 18.173 | 32.351 | 1.662 | 1.00 | 16.19 | O |
| ATOM | 1504 | O | HOH | C | 200 | −6.339 | 1.106 | −9.354 | 1.00 | 30.70 | O |
| ATOM | 1505 | O | HOH | C | 201 | 9.356 | 2.408 | −1.635 | 1.00 | 41.48 | O |
| ATOM | 1506 | O | HOH | C | 202 | −2.957 | 29.499 | −7.472 | 1.00 | 39.78 | O |
| ATOM | 1507 | O | HOH | C | 203 | 16.718 | 37.857 | −3.857 | 1.00 | 53.51 | O |
| ATOM | 1508 | O | HOH | C | 204 | 24.450 | 17.663 | −9.932 | 1.00 | 43.97 | O |
| ATOM | 1509 | O | HOH | C | 205 | 20.771 | 23.401 | 9.091 | 1.00 | 21.18 | O |
| ATOM | 1510 | O | HOH | C | 206 | 17.991 | 14.996 | 15.334 | 1.00 | 34.03 | O |
| ATOM | 1511 | O | HOH | C | 207 | 14.785 | 13.418 | 15.035 | 1.00 | 23.11 | O |
| ATOM | 1512 | O | HOH | C | 208 | 21.364 | 10.070 | 0.938 | 1.00 | 43.98 | O |
| ATOM | 1513 | O | HOH | C | 209 | 17.446 | 32.692 | 15.044 | 1.00 | 32.28 | O |
| ATOM | 1514 | O | HOH | C | 210 | −4.097 | −0.566 | −2.525 | 1.00 | 43.94 | O |
| ATOM | 1515 | O | HOH | C | 211 | −7.601 | 16.797 | −13.642 | 1.00 | 30.53 | O |
| ATOM | 1516 | O | HOH | C | 212 | −6.402 | 17.493 | −9.417 | 1.00 | 32.47 | O |
| ATOM | 1517 | O | HOH | C | 213 | 20.479 | 12.017 | −6.368 | 1.00 | 41.20 | O |
| ATOM | 1518 | O | HOH | C | 214 | 26.233 | 15.387 | −8.763 | 1.00 | 61.51 | O |
| ATOM | 1519 | O | HOH | C | 215 | 8.171 | 30.282 | 14.273 | 1.00 | 47.43 | O |
| ATOM | 1520 | O | HOH | C | 216 | −4.860 | 2.481 | −3.365 | 1.00 | 36.97 | O |
| ATOM | 1521 | O | HOH | C | 217 | 7.253 | 13.009 | −17.778 | 1.00 | 34.50 | O |
| ATOM | 1522 | O | HOH | C | 218 | 31.889 | 26.105 | 0.328 | 1.00 | 48.69 | O |
| ATOM | 1523 | O | HOH | C | 219 | 23.801 | 31.899 | −5.289 | 1.00 | 37.45 | O |
| ATOM | 1524 | O | HOH | C | 220 | 8.049 | 20.377 | 15.946 | 1.00 | 46.50 | O |
| ATOM | 1525 | O | HOH | C | 221 | 1.128 | 35.425 | −1.061 | 1.00 | 32.18 | O |
| ATOM | 1526 | O | HOH | C | 222 | 0.736 | 3.120 | −12.959 | 1.00 | 49.39 | O |
| ATOM | 1527 | O | HOH | C | 223 | 5.714 | 34.194 | −5.598 | 1.00 | 45.92 | O |
| ATOM | 1528 | O | HOH | C | 224 | 32.462 | 23.398 | −0.604 | 1.00 | 52.94 | O |
| ATOM | 1529 | O | HOH | C | 225 | 22.920 | 12.668 | −2.396 | 1.00 | 42.31 | O |
| ATOM | 1530 | O | HOH | C | 226 | −10.468 | 20.039 | −4.505 | 1.00 | 31.70 | O |
| ATOM | 1531 | O | HOH | C | 227 | −9.571 | 20.524 | 7.691 | 1.00 | 33.23 | O |
| ATOM | 1532 | O | HOH | C | 228 | −5.512 | 24.361 | −11.421 | 1.00 | 41.21 | O |
| ATOM | 1533 | O | HOH | C | 229 | 29.130 | 19.154 | −2.128 | 1.00 | 44.72 | O |
| ATOM | 1534 | O | HOH | C | 230 | 26.079 | 16.595 | −13.126 | 1.00 | 40.17 | O |
| ATOM | 1535 | O | HOH | C | 231 | 8.249 | 33.990 | −6.344 | 1.00 | 37.22 | O |
| ATOM | 1536 | O | HOH | C | 232 | −6.369 | 23.852 | −2.542 | 1.00 | 30.86 | O |
| ATOM | 1537 | O | HOH | C | 233 | 28.025 | 13.978 | −14.597 | 1.00 | 41.20 | O |
| ATOM | 1538 | O | HOH | C | 234 | 4.526 | 27.966 | −13.975 | 1.00 | 57.38 | O |
| ATOM | 1539 | O | HOH | C | 235 | 19.662 | 19.264 | 17.253 | 1.00 | 45.54 | O |
| ATOM | 1540 | O | HOH | C | 236 | 6.207 | 28.046 | 13.913 | 1.00 | 39.03 | O |
| ATOM | 1541 | O | HOH | C | 237 | 21.469 | 21.107 | 19.172 | 1.00 | 36.45 | O |
| ATOM | 1542 | O | HOH | C | 238 | 20.241 | 3.932 | 14.047 | 1.00 | 24.86 | O |
| ATOM | 1543 | O | HOH | C | 239 | 23.003 | 12.985 | −9.593 | 1.00 | 41.28 | O |
| ATOM | 1544 | O | HOH | C | 240 | 20.820 | 20.952 | 10.196 | 1.00 | 26.99 | O |
| ATOM | 1545 | O | HOH | C | 241 | −13.043 | 10.647 | −0.957 | 1.00 | 44.71 | O |
| ATOM | 1546 | O | HOH | C | 242 | −12.463 | 23.019 | 9.434 | 1.00 | 35.64 | O |
| ATOM | 1547 | O | HOH | C | 243 | 16.937 | 13.054 | 16.827 | 1.00 | 28.17 | O |
| ATOM | 1548 | O | HOH | C | 244 | 10.816 | 36.185 | −5.880 | 1.00 | 34.97 | O |
| ATOM | 1549 | O | HOH | C | 245 | 29.507 | 38.709 | 0.897 | 1.00 | 30.98 | O |
| ATOM | 1550 | O | HOH | C | 246 | −6.984 | 19.997 | −9.497 | 1.00 | 36.75 | O |
| ATOM | 1551 | O | HOH | C | 247 | 23.227 | 8.607 | 1.927 | 1.00 | 36.74 | O |
| ATOM | 1552 | O | HOH | C | 248 | 7.431 | 1.768 | −12.583 | 1.00 | 34.07 | O |
| ATOM | 1553 | O | HOH | C | 249 | −1.145 | 35.666 | −3.730 | 1.00 | 32.73 | O |
| ATOM | 1554 | O | HOH | C | 250 | −10.303 | 18.635 | 9.413 | 1.00 | 27.27 | O |
| ATOM | 1555 | O | HOH | C | 251 | 24.497 | 11.680 | −11.761 | 1.00 | 43.83 | O |
| ATOM | 1556 | O | HOH | C | 252 | 22.035 | −1.582 | 4.357 | 1.00 | 31.60 | O |
| ATOM | 1557 | O | HOH | C | 253 | 12.859 | 34.955 | −6.865 | 1.00 | 46.12 | O |
| ATOM | 1558 | O | HOH | C | 254 | −0.415 | 19.213 | 17.144 | 1.00 | 33.59 | O |
| ATOM | 1559 | O | HOH | C | 255 | −7.392 | −0.762 | −13.446 | 1.00 | 38.50 | O |
| ATOM | 1560 | O | HOH | C | 256 | 22.357 | 24.323 | 19.921 | 1.00 | 39.95 | O |
| ATOM | 1561 | O | HOH | C | 257 | 25.108 | 17.891 | 18.040 | 1.00 | 46.55 | O |
| ATOM | 1562 | O | HOH | C | 258 | −10.544 | 16.883 | 7.534 | 1.00 | 42.60 | O |
| ATOM | 1563 | O | HOH | C | 259 | −1.823 | 9.270 | 10.440 | 1.00 | 42.68 | O |
| ATOM | 1564 | O | HOH | C | 260 | 0.038 | 32.968 | −8.263 | 1.00 | 49.96 | O |
| ATOM | 1565 | O | HOH | C | 261 | −2.957 | 37.760 | −3.649 | 1.00 | 45.46 | O |
| ATOM | 1566 | O | HOH | C | 262 | 16.070 | 16.430 | 19.660 | 1.00 | 46.71 | O |
| ATOM | 1567 | O | HOH | C | 263 | −7.829 | 21.465 | −12.380 | 1.00 | 37.90 | O |
| ATOM | 1568 | O | HOH | C | 264 | −5.381 | 10.775 | 10.912 | 1.00 | 54.26 | O |
| ATOM | 1569 | O | HOH | C | 265 | 31.411 | 19.412 | −9.999 | 1.00 | 57.72 | O |
| ATOM | 1570 | O | HOH | C | 266 | 17.650 | −0.220 | 11.442 | 1.00 | 25.86 | O |
| ATOM | 1571 | O | HOH | C | 267 | 22.703 | 6.018 | 2.267 | 1.00 | 31.89 | O |
| ATOM | 1572 | O | HOH | C | 268 | 24.287 | −0.928 | 2.476 | 1.00 | 56.27 | O |
| ATOM | 1573 | O | HOH | C | 269 | 0.000 | 0.000 | 5.917 | 0.33 | 41.08 | O |
| ATOM | 1574 | O | HOH | C | 270 | 4.315 | 1.035 | −18.133 | 1.00 | 46.45 | O |
| ATOM | 1575 | O | HOH | C | 271 | 25.962 | 8.475 | 2.448 | 1.00 | 31.32 | O |
| ATOM | 1576 | O | HOH | C | 272 | 22.171 | 26.495 | 18.447 | 1.00 | 33.86 | O |
| ATOM | 1577 | O | HOH | C | 273 | 36.940 | 19.863 | −1.222 | 1.00 | 38.39 | O |
| ATOM | 1578 | O | HOH | C | 274 | 37.580 | 19.529 | 1.676 | 1.00 | 40.36 | O |
| ATOM | 1579 | O | HOH | C | 275 | −1.797 | 34.546 | −7.351 | 1.00 | 42.77 | O |
| ATOM | 1580 | O | HOH | C | 276 | −13.663 | 15.413 | 7.393 | 1.00 | 33.48 | O |
| ATOM | 1581 | O | HOH | C | 277 | 15.698 | 23.450 | 20.973 | 1.00 | 42.32 | O |
| ATOM | 1582 | O | HOH | C | 278 | −3.885 | 33.689 | −6.005 | 1.00 | 52.43 | O |
| ATOM | 1583 | O | HOH | C | 279 | 28.337 | 40.591 | 7.898 | 1.00 | 40.16 | O |
| ATOM | 1584 | O | HOH | C | 280 | 33.930 | 23.410 | −9.468 | 1.00 | 44.18 | O |
| ATOM | 1585 | O | HOH | C | 281 | 2.806 | 9.012 | 12.959 | 1.00 | 47.36 | O |
| ATOM | 1586 | O | HOH | C | 282 | 10.315 | 39.457 | 14.714 | 1.00 | 40.30 | O |
| ATOM | 1587 | O | HOH | C | 283 | −0.003 | 38.067 | −8.793 | 0.33 | 46.57 | O |
| ATOM | 1588 | O | HOH | C | 284 | −0.827 | 9.206 | 6.958 | 1.00 | 24.57 | O |
| ATOM | 1589 | O | HOH | C | 285 | 10.557 | 8.220 | −2.795 | 1.00 | 29.67 | O |
| ATOM | 1590 | O | HOH | C | 286 | 10.671 | 9.390 | 0.780 | 1.00 | 18.38 | O |
| ATOM | 1591 | O | HOH | C | 287 | 2.511 | 37.077 | 1.303 | 1.00 | 27.43 | O |
| ATOM | 1592 | O | HOH | C | 288 | 10.671 | 5.913 | 10.408 | 1.00 | 28.27 | O |
| ATOM | 1593 | O | HOH | C | 289 | 12.179 | 9.861 | −1.440 | 1.00 | 34.08 | O |
| ATOM | 1594 | O | HOH | C | 290 | 9.048 | 7.318 | −0.271 | 1.00 | 25.03 | O |
| ATOM | 1595 | O | HOH | C | 291 | −0.001 | 38.072 | 2.437 | 0.33 | 9.24 | O |

### Example 6

### Confirmation of Binding Abilities of Various Mutant Proteins

An interaction was confirmed by the method described in Example 1 except that mutant Hd3a and mutant GF14c were used. Mutant Hd3a (R55A, M63A, R64A, P96L, F103A, R132A, and R168A) and mutant GF14c (F200A, I204A, E212A, Y215A, and R226A) were produced using a Quickchange site-directed mutagenesis kit (Stratagene) according to the protocol of the kit. It should be noted that “R55A” means a mutant Hd3a protein having a substitution of arginine (R) at position 55 to alanine (A) in Hd3a.

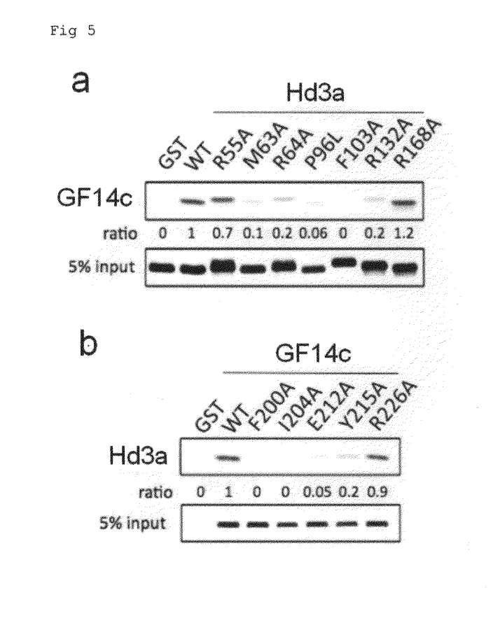

FIG. 5 show the results. Out of the Hd3a mutants, M63A, R64A, P96L, F103A, and R132A each exhibited a reduction in binding ability to GF14c. In particular, F103A exhibited a remarkable reduction in binding ability (FIG. 5a). Out of the GF14c mutants, F200A, I204A, E212A, and Y215A each exhibited a reduction in binding ability to Hd3a. In particular, F200A and I204A each exhibited a remarkable reduction in binding ability (FIG. 5b).

### Example 7

### Gel-Shift Assay of Florigen Activation Complex and C-Box DNA

An arabidopsis AP1 promoter-derived sequence (22 base pairs: 5′-CTTCACGAGACGTCGATAATCA-3′ (SEQ ID NO: 5)) was used as C-box DNA. C-box DNA used for the assay was prepared by chemical synthesis. A 100 mM Tris-borate buffer containing 0.2 mM EDTA, 1.5 mM MgCl₂, and 5% glycerol was used for the preparation of a florigen activation complex. A conjugate of the florigen activation complex (FD1-GF14-Hd3a) (sometimes referred to as “FAC”) and C-box DNA was generated by mixing 30 pmol of C-box DNA, 40 pmol of OsFD1, 40 pmol of GF14c, and 80 pmol of Hd3a with each other and subjecting the mixture to a reaction through incubation at 4° C. for 30 minutes. The generated conjugate was subjected to electrophoresis using a 10% acrylamide gel and detected by ethidium bromide staining.

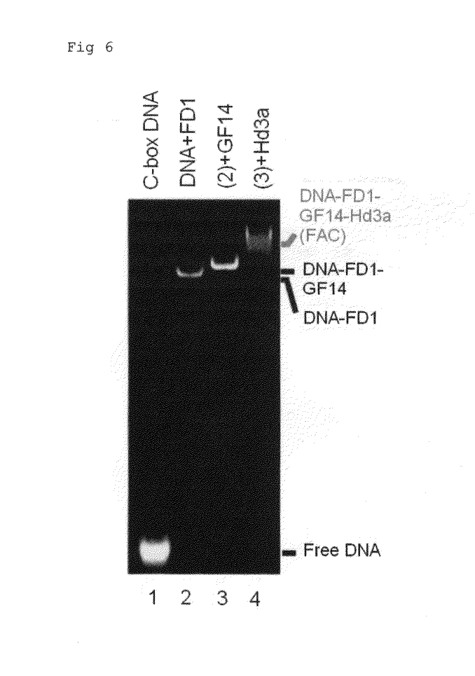

FIG. 6 shows the results. The florigen activation complex was found to bind to C-box DNA and form a stable complex on the DNA (lane 4). Hd3a is considered to form a florigen activation complex on a promoter and be responsible for the transcriptional activation of a gene necessary for flowering.

### Example 8

### Confirmation of Interactions Between Florigen and 14-3-3 Protein Isoforms

Interactions between respective proteins were confirmed using a yeast two-hybrid assay.

First, a plasmid was constructed as described below. Full-length cDNAs for OsGF14a (Os08g0480800), OsGF14b (Os04g0462500), OsGF14c (Os08g0430500), OsGF14d (Os11g0546900), OsGF14e (Os02g0580300), OsGF14f (Os03g0710800), OsGF14g (Os01g0209200), OsGF14h (Os11g0609600), and OsFD1 (Os09g0540800) were cloned by RT-PCR. The forward and reverse primers were designed based on genetic information in the rice DNA database. The full-length coding region was PCR-amplified and introduced into a pENTR/D-TOPO cloning vector (Invitrogen) to obtain entry clones.

The screening of Hd3a interactors was performed by the method described in Non Patent Literature 2. Yeast two-hybrid libraries were produced using total RNA extracted from wild-type leaf blades. After cDNA synthesis using a cDNA synthesis kit (Stratagene), the cDNAs were inserted into a pVP16 vector (Hollenberg et al., 1995) and introduced into a yeast L40 strain. As a bait, the full-length Hd3a ORF was cloned into a pBTM116 vector (Bartel et al., 1993). Screening was performed on an SC medium lacking histidine and containing 2.5 mM 3-aminotriazole (3-AT).

For an interaction assay, pBTM116 and pVP16 were converted to pBTM116-GW and pVP16-GW, respectively, using a Gateway vector conversion system (Invitrogen) according to the manufacturer's instructions. Yeast cells were grown at 30° C. for 5 days using an SC medium (without uracil, tryptophan, leucine, and histidine), the medium containing added histidine or 1 to 10 mM 3-aminotriazole (+His or +3-AT). The concentration of 3-AT was determined by a bait-prey combination.

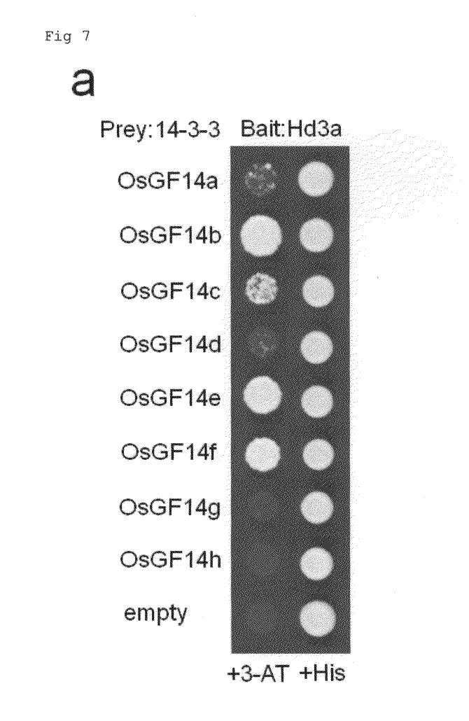

FIG. 7 shows the results. GF14b, c, e, and f (OsGF14b, c, e, and f) were each found to be exhibit an interaction with Hd3a. GF14b was used in the following examples.

### Example 9

### Confirmation of Sites Contributing to Interactions Among Three Proteins

Various mutant proteins were produced by constructing plasmids as described below. The introduction of amino-acid substitutions or deletions into OsGF14, OsFD1, and Hd3a was performed by PCR with KOD FX DNA polymerase (TOYOBO). The PCR-amplified fragments were cloned into a pENTR/D-TOPO cloning vector (Invitrogen) to obtain entry clones. Mutant proteins expressed in the obtained entry clones are as follows: S192A and S192E for OsFD1; R64A/R68A, I277A/L230A, F206A, I210A, E212A, E218A, Y221A, R232A, and D233A for OsGF14b; and R64G, R64A, R64K, T68I, P96L, F103A, R132A, R132K, R64G/R132A, and R64K/R132K for Hd3a. Interactions were confirmed in the same manner as in Example 8 using those entry clones.

It should be noted that, out of the mutant proteins, a protein indicated using “/” is a multiple mutant protein, and for example, “R64G/R132A” for Hd3a means a double-mutant Hd3a protein having a substitution of arginine (R) at position 64 to glycine (G) and a substitution of arginine (R) at position 132 to alanine (A).

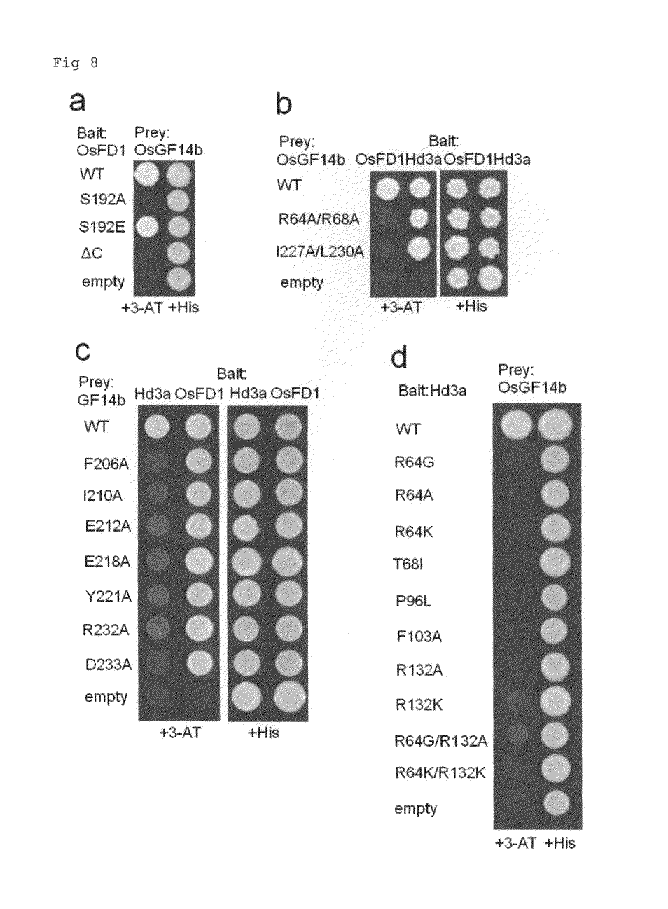

FIGS. 8a to 8d show the results.

The phosphorylation of serine at position 192 in OsFD1 was found to be important for an interaction between OsFD1 and OsGF14b (FIG. 8a).

Out of the mutant proteins for OsGF14b, in the mutant proteins having substitutions in F206, I210, E212, E218, Y221, R232, and D233, there were reductions in binding ability to Hd3a, whereas there was no influence on binding ability to OsFD1 (FIG. 8c).

Out of the mutant proteins for Hd3a, in the mutant proteins having substitutions in R64, T68, P96, F103, and 8132, there were remarkable reductions in binding ability to OsGF14b (FIG. 8d). Out of the mutant proteins for Hd3a, in the cases of P77L and R121H, there was no influence on binding ability to OsGF14b, suggesting that those sites were not important for binding to the 14-3-3 protein.

### Example 10

### Confirmation of Interaction in Shoot Apex of Rice

An interaction in the shoot apex of a rice plant was confirmed using an in vivo pull-down assay.

### (1) Used Plant Materials

Rice (Oryza sativa L. subsp. Japonica) variety Norin 8 was used as a wild-type. Transgenic rice (pHd3a::Hd3a::GFP, p35S::GFP, and prolC::Hd3a::GFP) were produced as described in Tamaki et al., Science 316, 1033 (2007) and Okano et al., Plant J. 53, 65 (2008). The transgenic rice was produced using Agrobacterium-mediated transformation of rice calli, as described in Hiei et al. Plant J. 6, 271 (1994), and a hygromycin-resistant plant was produced from the transformed calli. Transgene integration was further confirmed by PCR amplification of a hygromycin phosphotransferase (HPT) gene in genome DNA extracted from the produced plant. It should be noted that pHd3a::Hd3a::GFP refers to transgenic rice having introduced therein a gene in which a promoter region in Hd3a, a full-length coding region in Hd3a, and a coding region in GFP are conjugated to each other, and is hereinafter sometimes abbreviated as “HHG.” p35S::GFP refers to transgenic rice having introduced therein a gene in which a CaMV 35S promoter and a coding region in GFP are conjugated to each other, and is hereinafter sometimes abbreviated as “35S GFP.” prolC::Hd3a::GFP refers to transgenic rice having introduced therein a gene in which a promoter region in rolC, a full-length coding region in Hd3a, and a coding region in GFP are conjugated to each other.

### (3) In Vivo Pull-Down Assay

Samples of the shoot apical site were collected by microscopic dissection from HHG transgenic rice and 35S GFP transgenic rice. After grinding of the samples, the powder of each of the samples was dissolved in 100 μl of an extraction buffer (150 mM NaCl, 50 mM Tris, 0.1% Tween-20, 10% glycerol, 1 mM DTT, 1 mM Pefabloc SC (Roche), 1× Complete Proteinase Inhibitor Cocktail (Roche), and 1× Halt Phosphatase Inhibitor Cocktail (Pierce)), followed by mixing. After centrifugation (15 min, 4° C., 15,000 rcf), the supernatant was transferred to a tube, and the amount of a protein was measured by a Coomassie staining assay. An equivalent to the total amount of each of the proteins extracted from HHG transgenic rice and 35S GFP transgenic rice was incubated with 50 μl of anti-GFP MicroBeads (Miltenyi Biotec) according to the manufacturer's instructions except that the following modifications was made: a flow-through was collected and used for further analysis. The column was rinsed four times with 200 μl of an extraction buffer (150 mM NaCl, 50 mM Tris, 0.1% Tween-20, 10% glycerol, 1 mM DTT, 1 mM Pefabloc SC (Roche), 1× Complete Proteinase Inhibitor Cocktail (Roche), and 1× Halt Phosphatase Inhibitor Cocktail (Pierce)). After that, the column was washed once with 100 μl of a low ionic buffer (20 mM TrisHCl, pH 7.5). After that, the column was removed from the magnetic field, and the remaining protein was extracted with 50 μl of a lysis buffer. The eluate was separated by 12.5% SDS-PAGE and subjected to immunoblotting using a primary antibody (polyclonal rabbit anti-GFP antibody (Abcam)) and an anti-14-3-3 antibody (provided by Dr. Yohsuke Takahashi (Hiroshima University, Japan)). After washing with TBST, the membrane was incubated for 1 hour with anti-rabbit IgG conjugated to horseradish peroxidase (GE Healthcare). Detection was performed using enhanced chemiluminescence (ECL) protein gel blot detection reagents (GE Healthcare), and visualization was performed using an LAS-4000 mini Imager (Fujifilm).

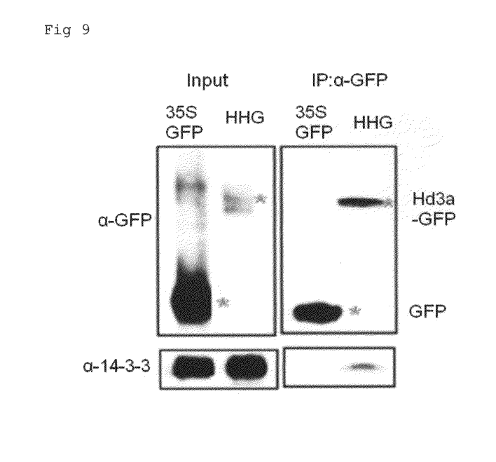

FIG. 9 shows the results. The left side (input) of FIG. 9 shows the results in the case of extracting a protein from shoot apex cells of transgenic rice expressing GFP or an Hd3a-GFP-fused protein (35S GFP transgenic rice or HHG transgenic rice) and subjecting the extract to immunoblotting using an anti-GFP antibody (α-GFP) and an anti-14-3-3 antibody (α-14-3-3). The right side (IP-GFP) of FIG. 9 shows the results in the case of subjecting an extract obtained by extracting a protein from shoot apex cells to immunoprecipitation (IP) with an anti-GFP antibody (α-GFP), followed by immunoblotting in the same manner as described above. When the Hd3a-GFP-fused protein was expressed (HHG extract), a 14-3-3 protein was observed during co-precipitation with the anti-GFP antibody. On the other hand, when GFP was expressed alone (35S GFP extract), a 14-3-3 protein was not observed during co-precipitation. This revealed that Hd3a interacted with the 14-3-3 protein in the shoot apex of rice.

### Example 11

### Subcellular Localization of Three Proteins and Complex Thereof

Subcellular localization and bimolecular fluorescence complementation (BiFC method) were performed as described below.

First, Hd3a, GF14b, OsFD1, and β-glucuronidase (GUS) coding regions were cloned into fluorescent protein expression vectors or BiFC vectors and purified using a Purelink Plasmid Midiprep Kit (Invitrogen). For Hd3a, GF14b, OsFD1, and mutant proteins thereof, full-length coding regions were used. A vector expressing GFP, CFP, or mCherry was used as each of the fluorescent protein expression vectors. A vector expressing a Vn or Vc tag was used as each of the BiFC vectors. Vn or Vc emits fluoresce as Venus (mVenus) when exists in proximity.

(1) The transformation of rice Oc protoplasts was performed by a method described in Kyozuka and Shimamoto 1991, Plant J. 6, 271 (1994), Non Patent Literature 1, or the like. Fluorescent protein expression vectors having introduced therein various proteins were introduced into a protoplast suspension (2×10⁷ protoplasts/ml) by a PEG method (PEG-mediated method). After incubation at 30° C. for 24 hours, transformed protoplasts were obtained and used for microscopic observation.

In a co-expression system of two kinds of proteins, 1 μg of a GFP-GF14b expression plasmid or an Hd3a-mCherry expression plasmid and 10 μg of an NLS-CFP, CFP-OsFD1, or CFP-OsFD1 S192A expression plasmid were co-transformed into protoplasts. NLS refers to a nuclear localization signal peptide.

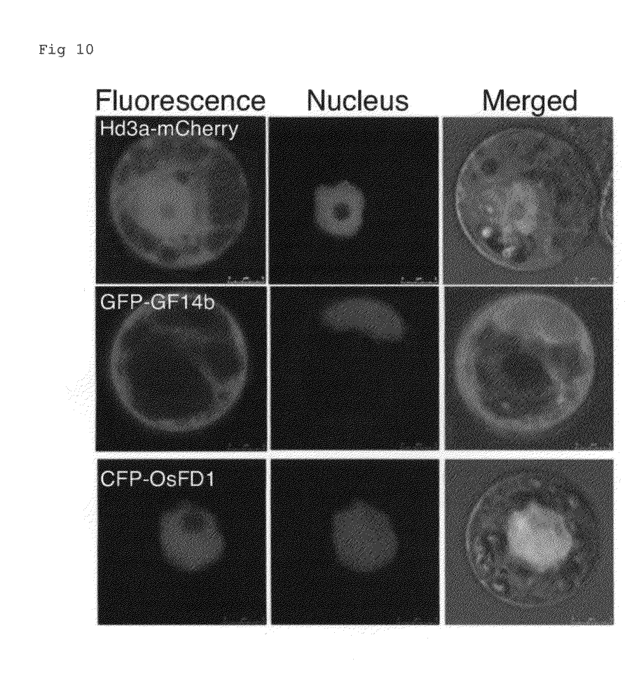

FIG. 10 shows the results. Fluorescence from Hd3a-mCherry was detected in both the cytoplasm and the nucleus, fluorescence from GFP-GF14b was detected in the cytoplasm, and fluorescence from CFP-OsFD1 was detected in the nucleus.

(2) In the same manner as in the method of (1), 5 μg of a Vn-fused protein expression vector and a Vc-fused protein expression vector were co-transformed in the BiFC method.

An mCherry expression plasmid was introduced simultaneously as a marker for transformation efficiency.

In order to quantify protein-protein interactions, the fluorescence intensities of mCherry and Venus from about 20 cells of various protoplasts having introduced therein expression plasmids for BiFC analysis (a Vn-fused protein expression vector, a Vc-fused protein expression vector, and an mCherry expression plasmid) were measured under the same microscope settings, and a Venus/mCherry value was calculated. In the experimental settings, a BiFC signal in cells showing Venus/mCherry values of >0.33 for an interaction between Hd3a and GF14b and Venus/mCherry values >0.4 for an interaction between GF14 band OsFD1 and an interaction between Hd3a and OsFD1 was recognized as reliable one. Therefore, the number of cells showing higher values than the above-mentioned values was scored.

FIG. 11 shows the results.

Vn or Vc is conjugated to the N-terminal and the C-terminal of Hd3a and GF14b and expressed in rice protoplasts. As a result, BiFC signals were confirmed in the cytoplasm for the interaction between Hd3a and GF14b, which were dependent on mutations in R64 and R132 of Hd3a (FIG. 11a). In the BiFC method, mutations in R58 and R62 of GF14b did not affect the interaction between Hd3a and GF14b.

Next, an interaction between GF14b and OsFD1 was confirmed. As a result, the interaction therebetween was not observed in the cytoplasm. The interaction between GF14b and OsFD1 was detected mainly in the nucleus, and only the localization of GF14b was confirmed in the cytoplasm (FIG. 11b). A mutation in 5192 of OsFD1 was found to inhibit the interaction with GF14b. Further, in the BiFC method, mutations in R58 and R132 of GF14b did not affect the interaction with OsFD1.

An interaction between Hd3a and OsFD1 was confirmed. As a result, a complex of Hd3a and OsFD1 was found to be present in the nucleus (FIG. 11c). Mutations in R64 and R132 of Hd3a or a mutation in 5192 of OsFD1 inhibited the interaction between Hd3a and OsFD1.

(3) In the same manner as in the method of (1), cells co-expressing OsFD1 in a BiFC system for confirming an interaction between Hd3a and GF14b were produced, and the amount of nuclear accumulation of the BiFC signal was examined. In order to co-express NLS-CFP and CFP-OsFD1 in the BiFC method, 10 μg of an NLS-CFP expression plasmid and a CFP-OsFD1 expression plasmid were used.

A method of calculating the amount of nuclear accumulation was established. First, the fluorescence intensities of Venus and mCherry in the nuclei of transformed cells were measured. After that, a value of (Venus in nucleus/mCherry in nucleus)/(Venus in whole cell/mCherry in whole cell) was calculated. This value indicates a ratio between the amounts of nuclear accumulation of Venus and mCherry. Those values and the corresponding confocal images obtained from each cell were compared to each other. As compared to mCherry distribution, values of more than 1.2 were recognized as nuclear localization, values of 0.8 to 1.2 were recognized as localization in both the nucleus and the cytoplasm, and values of less than 0.8 were recognized as cytoplasmic localization.

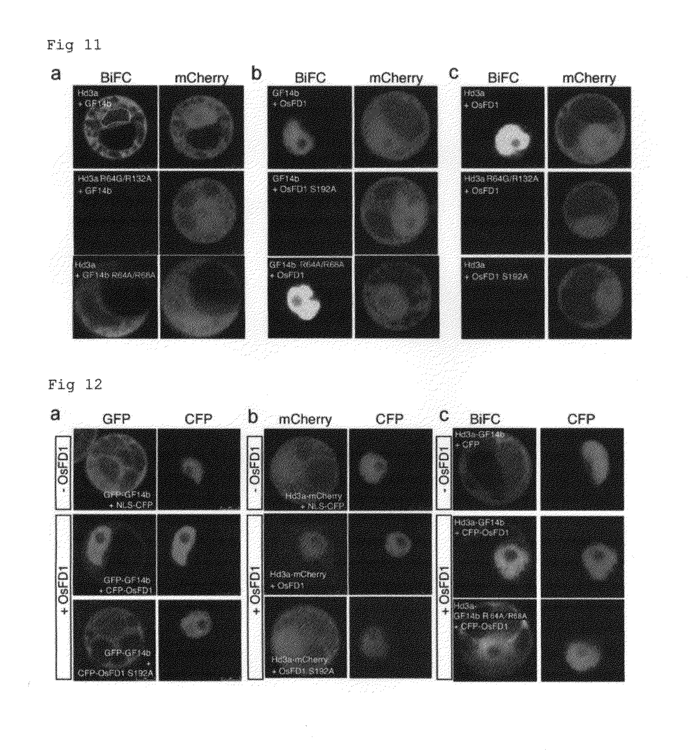

FIG. 12 shows the results.

In the case of co-expression of GFP-GF14b with NLS-CFP, fluorescence from GFP was confirmed in the cytoplasm. In the case of transient co-expression of GFP-GF14b with CFP-OsFD1, fluorescence from CFP was confirmed in the nucleus, and fluorescence from GFP was also observed in the nucleus, which remarkably differed from the case of the co-expression with NLS-CFP. On the other hand, in the case of co-expression of an OsFD1 mutant protein S192A with GFP-GF14b, the movement of fluorescence from GFP into the nucleus was not observed (FIG. 12a). This suggested that the localization of GF14b was transferred from the nucleus to the cytoplasm owing to the presence of OsFD1.

In the case of co-expression of Hd3a-mCherry with CFP-OsFD1, the amount of nuclear accumulation of mCherry increased as compared to the case of the co-expression with CFP alone. In the case of co-expression of an OsFD1 mutant protein S192A with Hd3a-mCherry, no increase in the amount of nuclear accumulation was found (FIG. 12b). Those results suggest that Hd3a and GF14b move from the cytoplasm to the nucleus to form a complex with OsFD1.

In order to confirm the influence of OsFD1 on the localization of a complex of Hd3a and GF14b, the BiFC method and CFP-OsFD1 were co-expressed. When OsFD1 was not expressed, the complex of Hd3a and GF14b (BiFC signal) was found to be present in the cytoplasm. In addition, when CFP-OsFD1 is co-expressed, the complex of Hd3a and GF14b was clearly observed predominantly in the nucleus (FIG. 12c). The movement of the complex of Hd3a and GF14b by OsFD1 co-expression was reduced by introducing mutations R58A/R62A into GF14b. For the localization of each protein in rice cells, it was suggested that the complex of Hd3a and GF14b was first formed in the cytoplasm, and the complex of Hd3a and GF14b interacted with OsFD1 in the nucleus through an interaction between GF14b and OsFD1.

### Example 12

### Influence of Interaction Among OsFD1, GF14b, and Hd3a on OsMADS15 Transcriptional Control

The influence of interactions among OsFD1, GF14b, and Hd3a on OsMADS15 transcriptional control was confirmed using a transient expression assay using protoplasts.

The transformation of rice Oc protoplasts was performed by a method described in Kyozuka and Shimamoto 1991, Plant J. 6, 271 (1994), Non Patent Literature 1, or the like. 8 μg of Hd3a expression vectors and 16 μg of OsFD1 expression vectors were introduced into 500 μl of a protoplast suspension (2×10⁷ protoplasts/ml) by a PEG method (PEG-mediated method). After incubation at 30° C. for 24 hours, the protoplast suspension was centrifuged and the cell pellet was frozen at −80° C. for RNA extraction.

pGII pUbq GW-T7 and pGII pUbq HA-GW were produced by inserting a ubiquitin promoter derived from maize, an NOS termination region (Miki and Shimamoto, Plant Cell Physiol. 45, 490 (2004)), and an attR recombination region with a T7 tag region or an HA tag region (Nakagawa et al., 2007) into a pGreen II vector (Hellens et al., Plant Mol. Biol. 42, 819 (2000)).

An Hd3a gene, an OsFD1 gene, or a mutant gene thereof incorporated in a pENTR D-TOPO vector was transformed into pGII pUbq GW-T7 and pGII pUbq HA-GW by a Gateway recombination method with Gateway BP clonase II (Invitrogen) (pGIIpUbqHd3a-T7, pGIIpUbqHA-OsFD1, pGIIpUbqHA-OsFD1, and pGIIpUbqHd3a-T7). Various Hd3a expression vectors and OsFD1 expression vectors were introduced into protoplasts to express various proteins, and the RNA expression amount of OsMADS15 was confirmed. It should be noted that OsGF14 is generally constantly expressed in all cells, and hence it is considered to be impossible to confirm its effect even when introduced.

(2) RNA extraction and real-time PCR analysis were performed as described below.

Leaf blades of wild-type and transgenic plants were harvested. Total RNA was extracted using a TRizol reagent (Invitrogen) according to the manufacturer's protocol. cDNA was synthesized from 1 or 2 μg of RNA using an oligo dT primer (21-mer) and reverse-transcribed with Superscript II reverse transcriptase (Invitrogen). 1 μl of cDNA was used for quantitative analysis of gene expression using SYBR Green PCR master mix (Applied Biosystems) with gene-specific primers. Data was collected using an ABI PRISM 7000 sequence detection system according to the manual. Primers for ubiquitin and OsMADS15 are as described in Komiya et al., Development 135, 767 (2008). Primers for GF14b and GF14e are as follows:

GF14b-realF
(SEQ ID NO: 4)
GCGGAAGAGATCAGGGAAG;
GF14b realR
(SEQ ID NO: 5)
CGACAACAATCACAGCCACA;
GF14e realF
(SEQ ID NO: 6)
CAAGGGCGAATCAGGAGA;
and
GF14e realR
(SEQ ID NO: 7)
TGAAACGATAACCCACAGCA.

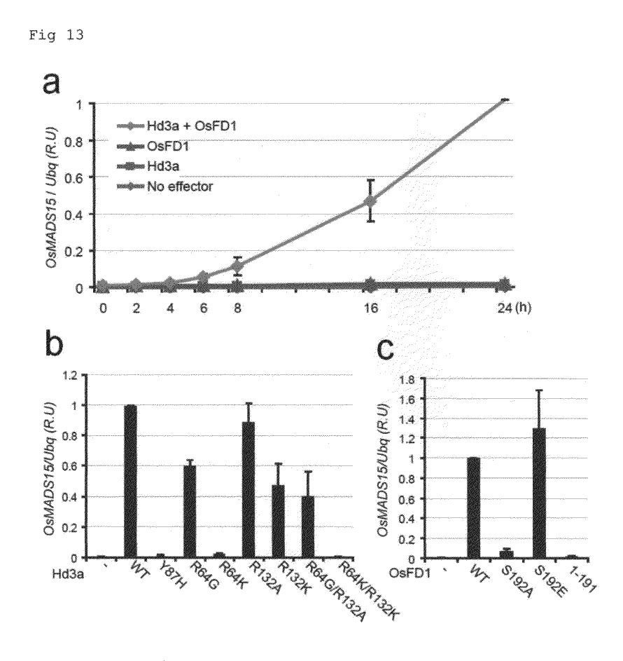

FIG. 13 shows the results.

The amount of OsMADS15 RNA increased with time after Hd3a and OsFD1 co-transformation. Hd3a or OsFD1 alone did not increase the RNA expression amount of OsMADS15. The OsMADS15 activation was found to start 4 hours after transformation, suggesting quick responsiveness between Hd3a and OsFD1 (FIG. 13a).

FIG. 13b shows the results of co-expression of various Hd3a mutants with OsFD1. Mutant Hd3a Y87H did not promote OsMADS15 transcription. Mutant Hd3a R64G and R132A slightly reduced OsMADS15 expression. Mutant Hd3a R64K and R64K/R132K remarkably reduced OsMADS15 expression. Those results suggest that the interaction between Hd3a and OsGF14 is required for OsMADS15 expression, and hydrophobicity, not a positive charge, of arginine is important for the interaction between Hd3a and OsGF14.

FIG. 13c shows the results of co-expression of various OsFD1 mutants with Hd3a. In the case where the SAP motif was deleted in OsFD1 (“1-191” in FIG. 13c: a fragment formed of residues 1-191, in which the subsequent amino acids were deleted) or in the case of a serine-to-alanine substitution in the SAP motif (S192A), the amount of OsMADS15 RNA accumulation was found to remarkably lower. In the case of a substitution of serine in the SAP motif to glutamic acid as a phospho-mimicking mutation (S192E), the amount of OsMADS15 RNA accumulation was not found to lower. Those facts suggest that the recognition of phosphorylated serine in the SAP motif in OsFD1 by the 14-3-3 protein is important for the formation of a florigen activation complex.

### Example 13-1

### Confirmation of Flowering Time in Transgenic Rice

In the same manner as in Example 10, transgenic rice was generated according to Table 3 and its flowering time was confirmed.

A plant was grown at a humidity of 70% in a chamber under short-day (SD) conditions (period including a light period at 30° C. for 10 hours and a dark period at 25° C. for 14 hours) or in a chamber under long-day (LD) conditions (period including a light period for 14 hours and a dark period for 10 hours). In the light period, photoirradiation was performed using a white-light fluorescent tube (400 to 700 nm, 100 μmol·m⁻²·s⁻¹). Rice suspension-cultured cells were maintained as described in Kyozuka and Shimamoto, 1991. A plant was grown at a humidity of 80% in a chamber under short-day (SD) conditions. The number of days to flowering was measured as the number of days to heading (heading stage) of a transgenic plant of Tₒ generation after transferred to SD conditions.

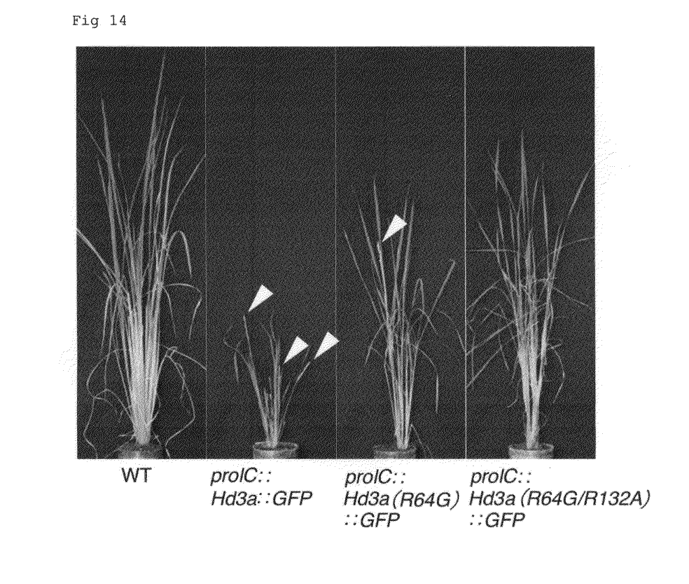

FIG. 14 and Table 3 show the results. An Hd3a-GFP protein derived from transgenic rice having introduced therein prolC::Hd3a::GFP moved from the shoot apex and strongly promoted flowering in the transgenic rice. prolC::Hd3a(R64G)::GFP (gene in which a promoter region in rolC, a coding region in R64G-mutant Hd3a, and a coding region in GFP were conjugated to each other) and prolC::Hd3a(R64G/R132A)::GFP (gene in which a promoter region in rolC, a coding region in R64G/R132A double-mutant Hd3a, and a coding region in GFP are conjugated to each other) were introduced into rice to confirm flowering time. As compared to transgenic rice having introduced therein prolC::Hd3a::GFP (19.6±12.3 days), transgenic rice having introduced therein prolC::Hd3a(R64G)::GFP (44.2±7.8 days) and transgenic rice having introduced therein prolC::Hd3a(R64G/R132A)::GFP (58.8±9.3 days) showed remarkably decreased promotion of flowering, and the latter was almost indistinguishable from a wild-type plant. Those facts suggest that an ability to promote flowering was lost by reduced affinity between Hd3a and 14-3-3.

Next, rice having introduced therein OsFD1 RNAi, transgenic rice expressing OsFD1, and transgenic rice having introduced therein mutant OsFD1 were analyzed. Rice having introduced therein OsFD1 RNAi flowered later than a wild-type. Transgenic rice expressing OsFD1 (pUbq::OsFD1) and transgenic rice having introduced therein mutant OsFD1 S192A (pUbq::OsFD1 (S192A)) were not affected in terms of flowering, and transgenic rice having introduced therein mutant OsFD1 S192E (pUbq::OsFD1 (S192E) (S192 phospho-mimicking)) promoted flowering as compared to a wild-type.

TABLE 3

| | Days to | |
|---|---|---|
| | flowering) | |
| Genotype | (days) | n |
| Wild-type | 54.9 ± 9.7 | 8 |
| prolC::Hd3a::GFP | 19.6 ± 12.3 | 13 |
| prolC::Hd3a(R64G)::GFP | 44.2 ± 7.8 | 6 |
| prolC::Hd3a(R64G/R132A)::GFP | 58.8 ± 9.3 | 21 |
| Wild-type* (wild-type) | 69.5 ± 6.0* | 4* |
| OsFD1 RNAi* | 74.6 ± 4.8* | 7* |
| pUbq::OsFD1 | 58.0 ± 6.1 | 3 |
| pUbq::OsFD1(S192A) | 61.7 ± 2.9 | 3 |
| pUbq::OsFD1(S192E) | 38.3 ± 4.6 | 8 |
| GF14b GF14e double RNAi | 60.1 ± 7.1 | 14 |
| pUbq::GF14b | 55.4 ± 10.9 | 5 |
| pUbq::NLS::GF14b | 51.0 ± 8.9 | 13 |
| pUbq::GF14e | 61.2 ± 11.0 | 6 |
| pUbq::NLS::GF14e | 47.7 ± 8.3 | 7 |

### Example 13-2

### Confirmation of Flowering Time in Transgenic Rice

Transgenic rice was generated in the same manner as in Example 13-1, and its flowering time was confirmed.

A plant (transgenic rice) was grown by the same technique as in Example 13-1, and the number of days to flowering was measured as the number of days to heading (heading stage) of a transgenic plant of T₀ generation after transferred to SD conditions.

FIG. 16 and Table 4 show the results. In the same manner as in Example 13-1, in prolC::Hd3a::GFP transgenic rice, the Hd3a-GFP protein moved from the shoot apex and strongly promote flowering in the transgenic rice. As compared to transgenic rice having introduced therein prolC::Hd3a::GFP, the promotion of flowering was not found in transgenic rice having introduced therein prolC::Hd3a(R64G/R132K)::GFP and transgenic rice having introduced therein prolC:Hd3a(F103A)::GFP (gene in which a promoter region in rolC, a coding region in F103A-mutant Hd3a, and a coding region in GFP were conjugated to each other).

TABLE 4

| | Days to | | |
|---|---|---|---|
| Genotype | Flowering | n | P |
| WT | 55.7 ± 9.4 | 9 | |
| prolC::Hd3a::GFP | 19.6 ± 12.3** | 13 | 0.00000018 |
| prolC::Hd3a(R64G)::GFP | 44.2 ± 7.8* | 6 | 0.024 |
| prolC::Hd3a(R64G/R132A)::GFP | 58.8 ± 9.3 | 21 | 0.42 |
| prolC::Hd3a(R64K/R132K)::GFP | 61.9 ± 9.7 | 15 | 0.13 |
| prolC::Hd3a(F103A)::GFP | 54.7 ± 7.9 | 7 | 0.83 |

*P < 0.05,
**P < 0.01

### INDUSTRIAL APPLICABILITY

The crystal of the present invention provides conformational information on the florigen activation complex important for the elucidation of a mechanism for controlling flowering of a plant, and may be used as a material for additional research on a mechanism for regulating flowering. Further, the flowering of a plant can be artificially and efficiently regulated based on the resultant conformational information, in particular, information on a binding site in the florigen activation complex. Based on the conformational information obtained in the present invention, a transgenic plant, which may be widely utilized for an increase in yield of an agricultural product, an improvement in efficiency of breeding, and the like, can be obtained. Further, a substance that regulates the flowering of a plant can be screened through the utilization of the conformational information on the present invention. Such substance can regulate the growth of a plant under various environments, and hence is considered to be beneficial from the agricultural viewpoint.

### (Sequence List)

Seq List¥YGP11-1003PCT_ST25.txt

LENGTHY TABLES
The patent application contains a lengthy table section. A copy of the table is available in electronic form from the USPTO web site (http://seqdata.uspto.gov/?pageRequest=docDetail&DocID=US20130019345A1). An electronic copy of the table will also be available from the USPTO upon request and payment of the fee set forth in 37 CFR 1.19(b)(3).

1. A method of regulating flowering of a plant, the method comprising at least one of promoting and suppressing formation of a florigen activation complex comprising a complex of a florigen, a 14-3-3 protein, and a bZIP transcription factor by affecting at least one of a binding site between the florigen and the 14-3-3 protein and a binding site between the 14-3-3 protein and the bZIP transcription factor in the florigen activation complex,
wherein:
the binding site between the florigen and the 14-3-3 protein comprises at least one site selected from the group consisting of sites of the florigen corresponding to D62, M63, R64, P96, T98, F103, and R132 in SEQ ID NO: 1 and sites of the 14-3-3 protein corresponding to F200, D201, I204, E212, Y215, R226, and D227 in SEQ ID NO: 2; and
the binding site between the 14-3-3 protein and the bZIP transcription factor comprises at least one site selected from the group consisting of sites of the 14-3-3 protein corresponding to K51, R58, F121, R131, L224, and D227 in SEQ ID NO: 2 and sites of the bZIP transcription factor corresponding to R189 to F195 in SEQ ID NO: 3.

2. A method of regulating flowering of a plant according to claim 1, wherein at least one of the promoting and suppressing of the formation of the florigen activation complex comprises generating a transformant comprising at least one of the following proteins (A) to (C):
(A) a florigen having a mutation in at least one binding site between the florigen and a 14-3-3 protein;
(B) a 14-3-3 protein having at least one of mutation in at least one binding site between a florigen and the 14-3-3 protein and at least one binding site between the 14-3-3 protein and a bZIP transcription factor; and
(C) a bZIP transcription factor having a mutation in at least one binding site between a 14-3-3 protein and the bZIP transcription factor.

3. A transformant, comprising at least one of the following proteins (A) to (C):
(A) a florigen having a mutation in at least one binding site between the florigen and a 14-3-3 protein;
(B) a 14-3-3 protein having at least one of mutation in at least one binding site between a florigen and the 14-3-3 protein and at least one binding site between the 14-3-3 protein and a bZIP transcription factor; and
(C) a bZIP transcription factor having a mutation in at least one binding site between a 14-3-3 protein and the bZIP transcription factor,
wherein:
the binding site between the florigen and the 14-3-3 protein comprises at least one site selected from the group consisting of sites of the florigen corresponding to D62, M63, R64, P96, T98, F103, and R132 in SEQ ID NO: 1 and sites of the 14-3-3 protein corresponding to F200, D201, I204, E212, Y215, R226, and D227 in SEQ ID NO: 2; and
the binding site between the 14-3-3 protein and the bZIP transcription factor comprises at least one site selected from the group consisting of sites of the 14-3-3 protein corresponding to K51, R58, F121, R131, L224, and D227 in SEQ ID NO: 2 and sites of the bZIP transcription factor corresponding to R189 to F195 in SEQ ID NO: 3.

4. A polynucleotide, which encodes at least one of the following proteins (A) to (C):
(A) a florigen having a mutation in at least one binding site between the florigen and a 14-3-3 protein;
(B) a 14-3-3 protein having at least one of mutation in at least one binding site between a florigen and the 14-3-3 protein and at least one binding site between the 14-3-3 protein and a bZIP transcription factor; and
(C) a bZIP transcription factor having a mutation in at least one binding site between a 14-3-3 protein and the bZIP transcription factor,
wherein:
the binding site between the florigen and the 14-3-3 protein comprises at least one site selected from the group consisting of sites of the florigen corresponding to D62, M63, R64, P96, T98, F103, and R132 in SEQ ID NO: 1 and sites of the 14-3-3 protein corresponding to F200, D201, I204, E212, Y215, R226, and D227 in SEQ ID NO: 2; and
the binding site between the 14-3-3 protein and the bZIP transcription factor comprises at least one site selected from the group consisting of sites of the 14-3-3 protein corresponding to K51, R58, F121, R131, L224, and D227 in SEQ ID NO: 2 and sites of the bZIP transcription factor corresponding to R189 to F195 in SEQ ID NO: 3.

5. A recombinant vector, comprising at least one of the polynucleotides of claim 4.

6. A method of screening a substance that regulates flowering of a plant, the method comprising any one of the following steps:
(1) a step including bringing a candidate substance into contact with any one of a florigen and a 14-3-3 protein, and bringing the candidate substance into contact with any one of the 14-3-3 protein and the florigen, respectively; and
(2) a step including bringing a candidate substance into contact with any one of a 14-3-3 protein to which a florigen is bound or unbound and a bZIP transcription factor, and bringing the candidate substance into contact with any one of the bZIP transcription factor and the 14-3-3 protein to which a florigen is bound or unbound, respectively.

7. A method of screening a substance that regulates flowering of a plant according to claim 6, the method further comprising the following step of:
selecting a candidate substance that bring at least one of promoting and inhibiting on binding in at least one of a binding site between the florigen and the 14-3-3 protein and a binding site between the 14-3-3 protein and the bZIP transcription factor in a presence of the candidate substance,
wherein:
the binding site between the florigen and the 14-3-3 protein comprises at least one site selected from the group consisting of sites of the florigen corresponding to D62, M63, R64, P96, T98, F103, and R132 in SEQ ID NO: 1 and sites of the 14-3-3 protein corresponding to F200, D201, I204, E212, Y215, R226, and D227 in SEQ ID NO: 2; and
the binding site between the 14-3-3 protein and the bZIP transcription factor comprises at least one site selected from the group consisting of sites of the 14-3-3 protein corresponding to K51, R58, F121, R131, L224, and D227 in SEQ ID NO: 2 and sites of the bZIP transcription factor corresponding to R189 to F195 in SEQ ID NO: 3.

8. A method of screening a substance that regulates flowering of a plant according to claim 6,
wherein:
the florigen comprises a florigen polypeptide fragment comprising an amino acid sequence which comprises at least a sequence of amino acids at positions 62 to 132, starts with one of amino acids at positions 1 to 6, and ends with one of amino acids at positions 165 to 177, in an amino acid sequence set forth in SEQ ID NO: 1;
the 14-3-3 protein comprises a 14-3-3 protein polypeptide fragment comprising an amino acid sequence which comprises at least a sequence of amino acids at positions 51 to 227, starts with one of amino acids at positions 1 to 5, and ends with one of amino acids at positions 230 to 256, in an amino acid sequence set forth in SEQ ID NO: 2; and
the bZIP transcription factor comprises a bZIP transcription factor polypeptide fragment comprising an amino acid sequence which comprises at least a sequence of amino acids at positions 189 to 195, starts with one of amino acids at positions 182 to 188, and ends with the amino acid at position 195, in an amino acid sequence set forth in SEQ ID NO: 3.

9. A polypeptide fragment, which is selected from the following:
(i) a novel florigen polypeptide fragment comprising an amino acid sequence which comprises at least a sequence of amino acids at positions 62 to 132, starts with one of amino acids at positions 1 to 6, and ends with one of amino acids at positions 165 to 177, in an amino acid sequence set forth in SEQ ID NO: 1;
(ii) a novel 14-3-3 protein polypeptide fragment comprising an amino acid sequence which comprises at least a sequence of amino acids at positions 51 to 227, starts with one of amino acids at positions 1 to 5, and ends with one of amino acids at positions 230 to 256, in an amino acid sequence set forth in SEQ ID NO: 2; and
(iii) a novel bZIP transcription factor polypeptide fragment comprising an amino acid sequence which comprises at least a sequence of amino acids at positions 189 to 195, starts with one of amino acids at positions 182 to 188, and ends with the amino acid at position 195, in OsFD1 set forth in SEQ ID NO: 3.

10. A polynucleotide, which encodes any one of the polypeptide fragments (i) to (iii) of claim 9.

11. A florigen activation complex, comprising a florigen polypeptide fragment bound to a bZIP transcription factor polypeptide fragment via a 14-3-3 protein polypeptide fragment,
wherein the florigen polypeptide fragment, the 14-3-3 protein polypeptide fragment, and the bZIP transcription factor polypeptide fragment comprise the polypeptide fragments (i) to (iii) of claim 9, respectively.

12. A crystal of a florigen activation complex, comprising a florigen bound to a bZIP transcription factor via a 14-3-3 protein.

13. A crystal of a florigen activation complex according to claim 12, wherein the crystal has a space group of P1, P6522, or P4 and lattice constants of a=74 to 158 Å, b=64 to 158 Å, c=96 to 500 Å, α=66 to 90°, β=85 to 90°, and γ=75 to 120°.

14. A method of producing the crystal of a florigen activation complex according to claim 12, the method comprising the steps of:
crystallizing a solution containing a complex of a florigen and a 14-3-3 protein by a vapor diffusion method using a precipitant solution containing at least as a precipitant 5 to 35 vol % polyethylene glycol;
collecting the resultant crystal; and
obtaining a crystal of a florigen activation complex by incubating the resultant crystal using a precipitant solution containing at least as a precipitant 5 to 35 vol % polyethylene glycol and containing a bZIP transcription factor.

15. A method of screening a substance that regulates flowering of a plant by at least one of designing and selecting a candidate substance having a function of regulating an activity of a florigen activation complex using a computer, the method comprising the steps of:
(a) causing storage means to store conformational information obtained from the crystal of a florigen activation complex of claim 12;
(b) causing deriving means to derive a three-dimensional conformation model based on the conformational information;
(c) causing calculation means to calculate an interatomic distance in the derived three-dimensional conformation model; and
(d) causing calculation means to calculate, based on the calculated interatomic distance, conformational information on a candidate substance being capable at least one of enhancing and inhibiting against at least one of binding between a florigen and a 14-3-3 protein and binding between a 14-3-3 protein and a bZIP transcription factor, and to bring at least one of designing and selecting the candidate substance.

16. A method of screening a substance that regulates flowering of a plant according to claim 7,
wherein:
the florigen comprises a florigen polypeptide fragment comprising an amino acid sequence which comprises at least a sequence of amino acids at positions 62 to 132, starts with one of amino acids at positions 1 to 6, and ends with one of amino acids at positions 165 to 177, in an amino acid sequence set forth in SEQ ID NO: 1;
the 14-3-3 protein comprises a 14-3-3 protein polypeptide fragment comprising an amino acid sequence which comprises at least a sequence of amino acids at positions 51 to 227, starts with one of amino acids at positions 1 to 5, and ends with one of amino acids at positions 230 to 256, in an amino acid sequence set forth in SEQ ID NO: 2; and
the bZIP transcription factor comprises a bZIP transcription factor polypeptide fragment comprising an amino acid sequence which comprises at least a sequence of amino acids at positions 189 to 195, starts with one of amino acids at positions 182 to 188, and ends with the amino acid at position 195, in an amino acid sequence set forth in SEQ ID NO: 3.

17. A method of producing the crystal of a florigen activation complex according to claim 13, the method comprising the steps of:
crystallizing a solution containing a complex of a florigen and a 14-3-3 protein by a vapor diffusion method using a precipitant solution containing at least as a precipitant 5 to 35 vol % polyethylene glycol;
collecting the resultant crystal; and
obtaining a crystal of a florigen activation complex by incubating the resultant crystal using a precipitant solution containing at least as a precipitant 5 to 35 vol % polyethylene glycol and containing a bZIP transcription factor.

18. A method of screening a substance that regulates flowering of a plant by at least one of designing and selecting a candidate substance having a function of regulating an activity of a florigen activation complex using a computer, the method comprising the steps of:
(a) causing storage means to store conformational information obtained from the crystal of a florigen activation complex of claim 13;
(b) causing deriving means to derive a three-dimensional conformation model based on the conformational information;
(c) causing calculation means to calculate an interatomic distance in the derived three-dimensional conformation model; and
(d) causing calculation means to calculate, based on the calculated interatomic distance, conformational information on a candidate substance being capable at least one of enhancing and inhibiting against at least one of binding between a florigen and a 14-3-3 protein and binding between a 14-3-3 protein and a bZIP transcription factor, and to bring at least one of designing and selecting the candidate substance.
